(19) United States
(12) Patent Application Publication
(10) Pub. No.: US 2021/0196678 A1
(43) Pub. Date: Jul. 1, 2021

(54) SELECTIVE ANDROGEN RECEPTOR DEGRADER (SARD) LIGANDS AND METHODS OF USE THEREOF

(71) Applicants: University of Tennessee Research Foundation, Knoxville, TN (US); GTX, INC., Memphis, TN (US)

(72) Inventors: Ramesh NARAYANAN, Cordova, TN (US); Duane D. MILLER, Collierville, TN (US); Thamarai PONNUSAMY, Memphis, TN (US); Dong-Jin HWANG, Arlington, TN (US); Yali HE, Germantown, TN (US)

(73) Assignee: University of Tennessee Research Foundation, Knoxville, TN (US)

(21) Appl. No.: 17/055,988

(22) PCT Filed: May 16, 2019
(86) PCT No.: PCT/US2019/032751

§ 371 Date: Nov. 16, 2020

Related U.S. Application Data
(60) Provisional application No. 62/727,309, filed on Sep. 5, 2018.
(60) Provisional application No. 62/672,558, filed on May 16, 2018.
(60) Provisional application No. 62/672,564, filed on May 16, 2018.

(51) Int. Cl. A61K 31/404; A61K 31/416; A61K 31/4192; A61K 31/4155; A61K 31/277; A61K 31/336; A61K 31/4439; A61K 45/06; A61P 35/00

(52) U.S. Cl. CPC A61K 31/404; A61K 31/416; A61K 31/4192; A61K 31/4155; A61P 35/00; A61K 31/336; A61K 31/4439; A61K 45/06; A61K 31/277

(57) ABSTRACT

This invention is directed to selective androgen receptor degrader (SARD) compounds including heterocyclic rings and pharmaceutical compositions and uses thereof in treating prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor-full length (AR-FL) including pathogenic or resistance mutations, AR-splice variants (AR-SV), and pathogenic polyglutamine (polyQ) polymorphisms of AR in a subject.

### FIELD OF THE INVENTION

This invention is directed to selective androgen receptor degrader (SARD) compounds including heterocyclic rings and pharmaceutical compositions and uses thereof in treating prostate cancer, advanced prostate cancer, castration resistant prostate cancer, triple negative breast cancer, other cancers expressing the androgen receptor, androgenic alopecia or other hyperandrogenic dermal diseases, Kennedy's disease, amyotrophic lateral sclerosis (ALS), abdominal aortic aneurysm (AAA), and uterine fibroids, and to methods for reducing the levels of androgen receptor-full length (AR-FL) including pathogenic or resistance mutations, AR-splice variants (AR-SV), and pathogenic polyglutamine (polyQ) polymorphisms of AR in a subject.

### BACKGROUND OF THE INVENTION

Prostate cancer (PCa) is one of the most frequently diagnosed noncutaneous cancers among men in the US and is the second most common cause of cancer deaths with more than 200,000 new cases and over 30,000 deaths each year in the United States. PCa therapeutics market is growing at an annual rate of 15-20% globally.

Androgen-deprivation therapy (ADT) is the standard of treatment for advanced PCa. Patients with advanced prostate cancer undergo ADT, either by luteinizing hormone releasing hormone (LHRH) agonists, LHRH antagonists or by bilateral orchiectomy. Despite initial response to ADT, disease progression is inevitable and the cancer emerges as castration-resistant prostate cancer (CRPC). Up to 30% of patients with prostate cancer that undergo primary treatment by radiation or surgery will develop metastatic disease within 10 years of the primary treatment. Approximately 50,000 patients a year will develop metastatic disease, which is termed metastatic CRPC (mCRPC).

Patients with CRPC have a median survival of 12-18 months. Though castration-resistant, CRPC is still dependent on the androgen receptor (AR) signaling axis for continued growth. The primary reason for CRPC re-emergence is re-activation of AR by alternate mechanisms such as: 1) intracrine androgen synthesis, 2) AR splice variants (AR-SV), e.g., that lack ligand binding domain (LBD), 3) AR-LBD mutations with potential to resist AR antagonists (i.e., mutants that are not sensitive to inhibition by AR antagonists, and in some cases AR antagonists act as agonists of the AR bearing these LBD mutations), 4) amplifications of the AR gene within the tumor (e.g., as driven by the fusion of other genes such as the ETS family of transcription factors (see for example PMID: 20478527, 30033370), and 5) rearrangements of the AR gene within the tumor, e.g., as described in PMID: 27897170. A critical barrier to progress in treating CRPC is that AR signaling inhibitors such as darolutamide, enzalutamide, apalutamide, bicalutamide, and abiraterone, acting through the LBD, fail to inhibit growth driven by the N-terminal domain (NTD)-dependent constitutively active AR-SV such as AR-V7, the most prominent AR-SV. Recent high-impact clinical trials with enzalutamide and abiraterone in CRPC patients demonstrated that just 13.9% of AR-V7-positive patients among 202 patients starting treatment with enzalutamide (Xtandi) or abiraterone acetate (Zytiga) had PSA responses to either of the treatments (Antonarakis E S, Lu C, Luber B, et al. J. Clin. Oncol. 2017 Apr. 6. doi: 10.1200/JCO.2016.70.1961), indicating the requirement for next generation AR antagonists that target AR-SVs. In addition, a significant number of CRPC patients are becoming refractory to abiraterone or enzalutamide and apalutamide, emphasizing the need for next generation AR antagonists.

Current evidences demonstrate that CRPC growth is dependent on constitutively active AR including AR-SV's that lack the LBD such as AR-V7 and therefore cannot be inhibited by conventional antagonists. AR inhibition and degradation through binding to a domain that is distinct from the AR LBD provides alternate strategies to manage CRPC.

Molecules that degrade the AR prevent any inadvertent AR activation through growth factors or signaling pathways, or promiscuous ligand-dependent activation. In addition, molecules that inhibit the constitutive activation of AR-SVs are extremely important to provide extended benefit to CRPC patients.

Currently only a few chemotypes are known to degrade AR which include the SARDs ARN-509, AZD-3514, and ASC-J9. However, these molecules degrade AR indirectly at much higher concentrations than their binding coefficient and they fail to degrade the AR-SVs that have become in recent years the primary reason for resurgence of treatment-resistant CRPC.

This invention describes novel AR antagonists with unique pharmacology that strongly (high potency and efficacy) and selectively bind AR (better than known antagonists in some cases; bind to LBD and/or NTD), antagonize AR, and degrade AR full length (AR-FL) and AR-SV. Selective androgen receptor degrader (SARD) compounds possess dual degradation and AR-SV inhibitory functions and hence are distinct from any available CRPC therapeutics. These novel selective androgen receptor degrader (SARD) compounds inhibit the growth of PCa cells and tumors that are dependent on AR-FL and AR-SV for proliferation.

SARDs have the potential to evolve as new therapeutics to treat CRPCs that are untreatable with any other antagonists. This unique property of degrading AR-SV has extremely important health consequences for prostate cancer. Till date only one series of synthetic molecules (EPI-001, EPI-506, etc.) and some marine natural products such as the sinkotamides and glycerol ether Naphetenone B, are reported to bind to AR-NTD and inhibit AR function and PCa cell growth, albeit at lower affinity and inability to degrade the receptor. The SARDs reported herein also bind to AR-NTD and inhibit NTD-driven (e.g., ligand independent) AR activity.

The positive correlation between AR and PCa and the lack of a fail-safe AR antagonist, emphasizes the need for molecules that inhibit AR function through novel or alternate mechanisms and/or binding sites, and that can elicit antagonistic activities within an altered cellular environment.

Although traditional antiandrogens such as enzalutamide, apalutamide, bicalutamide and flutamide and androgen deprivation therapies (ADT) were approved for use in prostate cancer, there is significant evidence that antiandrogens could also be used in a variety of other hormone dependent and hormone independent cancers. For example, antiandrogens have been tested in breast cancer (enzalutamide; Breast Cancer Res. (2014) 16(1): R7), non-small cell lung cancer (shRNAi AR), renal cell carcinoma (ASC-J9), partial androgen insensitivity syndrome (PAIS) associated malignancies such as gonadal tumors and seminoma, advanced pancreatic cancer (World J. Gastroenterology 20(29), 9229), cancer of the ovary, fallopian tubes, or peritoneum, cancer of the salivary gland (Head and Neck (2016) 38, 724-731; ADT was tested in AR-expressing recurrent/metastatic salivary gland cancers and was confirmed to have benefit on progression free survival and overall survival endpoints), bladder cancer (Oncotarget 6(30), 29860-29876); Int J. Endocrinol (2015), Article ID 384860), pancreatic cancer, lymphoma (including mantle cell), and hepatocellular carcinoma. Use of a more potent antiandrogen such as a SARD in these cancers may more efficaciously treat the progression of these and other cancers. Other cancers may also benefit from SARD treatment such as breast cancer (e.g., triple negative breast cancer (TNBC)), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer.

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, there is evidence that certain TNBC's may be supported by androgen independent activation of AR-SVs lacking the LBD or androgen-dependent activation of AR full length. As such, enzalutamide, apalutamide, and other LBD-directed traditional AR antagonists would not be able to antagonize AR-SVs in these TNBC's. However, SARDs of this invention which are capable of destroying AR-SVs (see Table 1 and Examples 2 and 7) through a binding site in the NTD of AR (see Example 9 of US2017-0368003) would be able to antagonize AR including AR-SV observed in TNBC patient derived xenograpfts and provide an anti-tumor effect, as shown in Example 8 of US2017-0368003.

Traditional antiandrogens such as bicalutamide and flutamide were approved for use in prostate cancer. Subsequent studies have demonstrated the utility of antiandrogens (e.g., flutamide, spironolactone, cyproterone acetate, finasteride and chlormadinone acetate) in androgen-dependent dermatological conditions such as androgenic alopecia (male pattern baldness), acne vulgaris, and hirsutism (e.g., in female facial hair). Prepubertal castration prevents sebum production and androgenic alopecia but this can be reversed by use of testosterone, suggesting its androgen-dependence.

The AR gene has a polymorphism of glutamine repeats (polyQ) within exon 1 which when shortened may augment AR transactivation (i.e., hyperandrogenism). It has been found that shortened polyQ polymorphisms are more common in people with alopecia, hirsutism, and acne. Classic antiandrogens are undesirable for these purposes because they are ineffective through dermal dosing and their long-term systemic use raises the risks of untoward sexual effects such as gynecomastia and impotence. Further, similar to CPRC discussed above, inhibition of ligand-dependent AR activity alone may not be sufficient as AR can be activated by various cellular factors other than the endogeneous androgens testosterone (T) and dihydrotestosterone (DHT), such as growth factors, kinases, co-activator overexpression and/or promiscuous activation by other hormones (e.g., estrogens or glucocorticoids). Consequently, blocking the binding of T and DHT to AR with a classical antiandrogen may not be sufficient to have the desired efficacy.

An emerging concept is the topical application of a SARD to destroy the AR locally to the affected areas of the skin or other tissue without exerting any systemic antiandrogenism. For this use, a SARD that does not penetrate the skin or is rapidly metabolized would be preferrable.

Supporting this approach is the observation that cutaneous wound healing has been demonstrated to be suppressed by androgens. Castration of mice accelerates cutaneous wound healing while attenuating the inflammation in the wounds. The negative correlation between androgen levels and cutaneous healing and inflammation, in part, explains another mechanism by which high levels of endogenous androgens exacerbate hyperandrogenic dermatological conditions. Further, it provides a rationale for the treatment of wounds such as diabetic ulcers or even trauma, or skin disorders with an inflammatory component such as acne or psoriasis, with a topical SARD.

Androgenic alopecia occurs in ˜50% of Caucasian males by midlife and up to 90% by 80 years old. Minoxidil (a topical vasodilator) and finasteride (a systemic 5alpha reductase type II inhibitor) are FDA approved for alopecia but require 4-12 months of treatment to produce a therapeutic effect and only arrest hair loss in most with mild to moderate hair regrowth in 30-60%. Since currently available treatments have slow and limited efficacy that varies widely between individuals, and produce unwanted sexual side effects, it is important to find a novel approach to treat androgenic alopecia and other hyperandrogenic dermatologic diseases.

Amyotrophic lateral sclerosis (ALS) is a fatal neurodegenerative disease characterized by selective loss of upper and lower motor neurons and skeletal muscle atrophy. Epidemiologic and experimental evidence suggest the involvement of androgens in ALS pathogenesis (“Anabolic/androgenic steroid nandrolone exacerbates gene expression modifications induced by mutant SOD1 in muscles of mice models of amyotrophic lateral sclerosis.” Galbiati M, Onesto E, Zito A, Crippa V, Rusmini P, Mariotti R, Bentivoglio M, Bendotti C, Poletti A. Pharmacol. Res. 2012, 65(2), 221-230), but the mechanism through which androgens modify the ALS phenotype is unknown. A transgenic animal model of ALS demonstrated improved survival upon surgical castration (i.e., androgen ablation). Treatment of these castrated animals with the androgen agonist nandrolone decanoate worsened disease manifestations. Castration reduces the AR level, which may be the reason for extended survival. The survival benefit is reversed by androgen agonist (“Androgens affect muscle, motor neuron, and survival in a mouse model of SOD1-related amyotrophic lateral sclerosis.” Aggarwal T, Polanco M J, Scaramuzzino C, Rocchi A, Milioto C, Emionite L, Ognio E, Sambataro F, Galbiati M, Poletti A, Pennuto M. Neurobiol. Aging. 2014 35(8), 1929-1938). Notably, stimulation with nandrolone decanoate promoted the recruitment of endogenous androgen receptor into biochemical complexes that were insoluble in sodium dodecyl sulfate, a finding consistent with protein aggregation. Overall, these results shed light on the role of androgens as modifiers of ALS pathogenesis via dysregulation of androgen receptor homeostasis. Antiandrogens should block the effects of nandrolone undecanoate or endogeneous androgens and reverse the toxicities due to AR aggegregation. Further, an antiandrogen that can block action of LBD-dependent AR agonists and concomitantly lower AR protein levels, such as the SARDs of this invention, would be therapeutic in ALS. Riluzole is an available drug for ALS treatment, however, it only provides short-term effects. There is an urgent need for drugs that extend the survival of ALS patients.

Androgen receptor action promotes uterine proliferation. Hyperandrogenicity of the short polyQ A R has been associated with increased leiomyoma or uterine fibroids. (Hsieh Y Y, Chang C C, Tsai F J, Lin C C, Yeh L S, Peng C T. J. Assist. Reprod. Genet. 2004, 21(12), 453-457). A separate study of Brazilian women found that shorter and longer [CAG](n) repeat alleles of A R were exclusive to the leiomyoma group in their study (Rosa F E, Canevari Rde A, Ambrosio E P, Ramos Cirilo P D, Pontes A, Rainho C A, Rogatto S R. Clin. Chem. Lab. Med. 2008, 46(6), 814-823). Similarly, in Asian Indian women long polyQ AR was associated with endometriosis and leiomyoma and can be regarded as high-risk markers. SARDs could be used in women with uterine fibroids, especially those expressing shorter and longer [CAG](n) repeat alleles, to treat existing uterine fibroids, prevent worsening of fibroids and/or ameliorate carcinogenicity associated with fibroids.

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, Salmon M, Pope N H, Lu G, Su G, Meher A, Ailawadi G, Upchurch G R Jr. J Vasc Surg (2016) 63(6):1602-1612) showed that flutamide (50 mg/kg) or ketoconazole (150 mg/kg) attenuated porcine pancreatic elastase (0.35 U/mL) induced AAA by 84.2% and 91.5% compared to vehicle (121%). Further AR −/− mice showed attenuated AAA growth (64.4%) compared to wildtype (both treated with elastase). Correspondingly, administration of a SARD to a patient suffering from an AAA may help reverse, treat or delay progression of AAA to the point where surgery is needed.

X-linked spinal-bulbar muscular atrophy (SBMA—also known as Kennedy's disease) is a muscular atrophy that arises from a defect in the androgen receptor gene on the X chromosome. Proximal limb and bulbar muscle weakness results in physical limitations including dependence on a wheelchair in some cases. The mutation results in a protracted polyglutamine tract added to the N-terminal domain of the androgen receptor (polyQ AR). Binding and activation of this lengthened polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci. 2016 58(3), 343-347). These steps are required for pathogenesis and result in partial loss of transactivation function (i.e., an androgen insensitivity) and a poorly understood neuromuscular degeneration. Support of the use of antiandrogens comes in a report in which the antiandrogen flutamide protects male mice from androgen-dependent toxicity in three models of spinal bulbar muscular atrophy (Renier K J, Troxell-Smith S M, Johansen J A, Katsuno M, Adachi H, Sobue G, Chua J P, Sun Kim H, Lieberman A P, Breedlove S M, Jordan C L. Endocrinology 2014, 155(7), 2624-2634). Currently there are no disease-modifying treatments but rather only symptom directed treatments. Efforts to target the polyQ AR of Kennedy's disease as the proximal mediator of toxicity by harnessing cellular machinery to promote its degradation, i.e., through the use of a SARD, hold promise for therapeutic intervention. Selective androgen receptor degraders such as those reported herein bind to and degrade all androgen receptors tested (full length, splice variant, antiandrogen resistance mutants, etc.) so degradation of polyQ AR polymorphism is also expected, indicating that they are promising leads for treatment of SBMA.

Here selective androgen receptor degrader (SARD) compounds are described that may bind to the LBD and/or an alternate binding and degradation domain (BDD) located in the NTD, antagonize AR, and degrade AR thereby blocking ligand-dependent and ligand-independent AR activities. This novel mechanism produces improved efficacy when dosed systemically (e.g., for prostate cancer) or topically (e.g., dermatological diseases).

### SUMMARY OF THE INVENTION

One embodiment of the invention encompasses a selective androgen receptor degrader (SARD) compound, or its isomer, pharmaceutically acceptable salt, pharmaceutical product, polymorph, hydrate or any combination thereof, wherein said SARD compound is represented by a compound of the following structures:

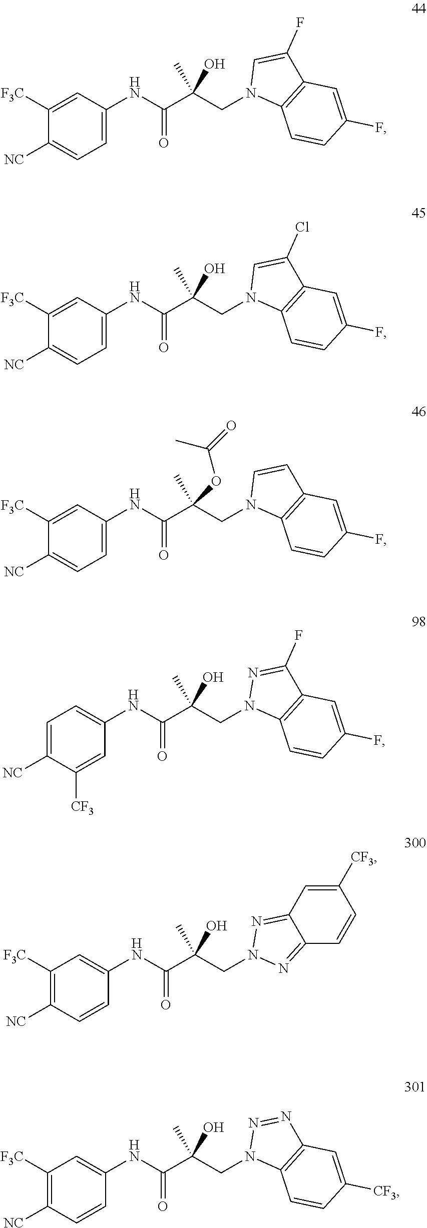 [SMILES: C[C@](O)(Cn1cc(F)c2cc(F)ccc21)C(=O)Nc1ccc(C#N)c(C(F)(F)F)c1 ; C[C@](O)(Cn1cc(Cl)c2cc(F)ccc21)C(=O)Nc1ccc(C#N)c(C(F)(F)F)c1 ; CC(=O)O[C@@](C)(Cn1ccc2cc(F)ccc21)C(=O)Nc1ccc(C#N)c(C(F)(F)F)c1 ; C[C@](O)(Cn1nc(F)c2cc(F)ccc21)C(=O)Nc1ccc(C#N)c(C(F)(F)F)c1 ; C[C@](O)(Cn1nc2ccc(C(F)(F)F)cc2n1)C(=O)Nc1ccc(C#N)c(C(F)(F)F)c1 ; C[C@](O)(Cn1nnc2cc(C(F)(F)F)ccc21)C(=O)Nc1ccc(C#N)c(C(F)(F)F)c1]

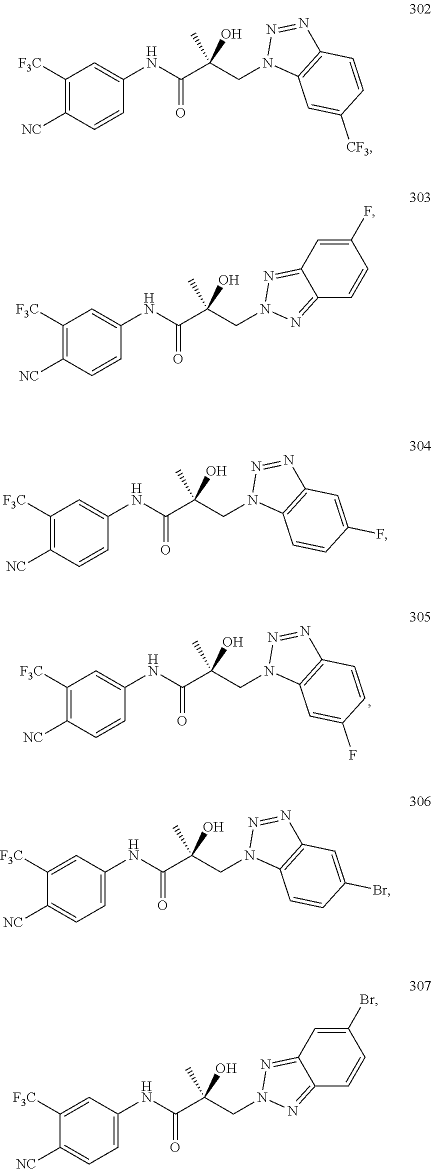 [SMILES: C[C@](O)(Cn1nnc2ccc(C(F)(F)F)cc21)C(=O)Nc1ccc(C#N)c(C(F)(F)F)c1 ; C[C@](O)(Cn1nc2ccc(F)cc2n1)C(=O)Nc1ccc(C#N)c(C(F)(F)F)c1 ; C[C@](O)(Cn1nnc2cc(F)ccc21)C(=O)Nc1ccc(C#N)c(C(F)(F)F)c1 ; C[C@](O)(Cn1nnc2ccc(F)cc21)C(=O)Nc1ccc(C#N)c(C(F)(F)F)c1 ; C[C@](O)(Cn1nnc2cc(Br)ccc21)C(=O)Nc1ccc(C#N)c(C(F)(F)F)c1 ; C[C@](O)(Cn1nc2ccc(Br)cc2n1)C(=O)Nc1ccc(C#N)c(C(F)(F)F)c1]

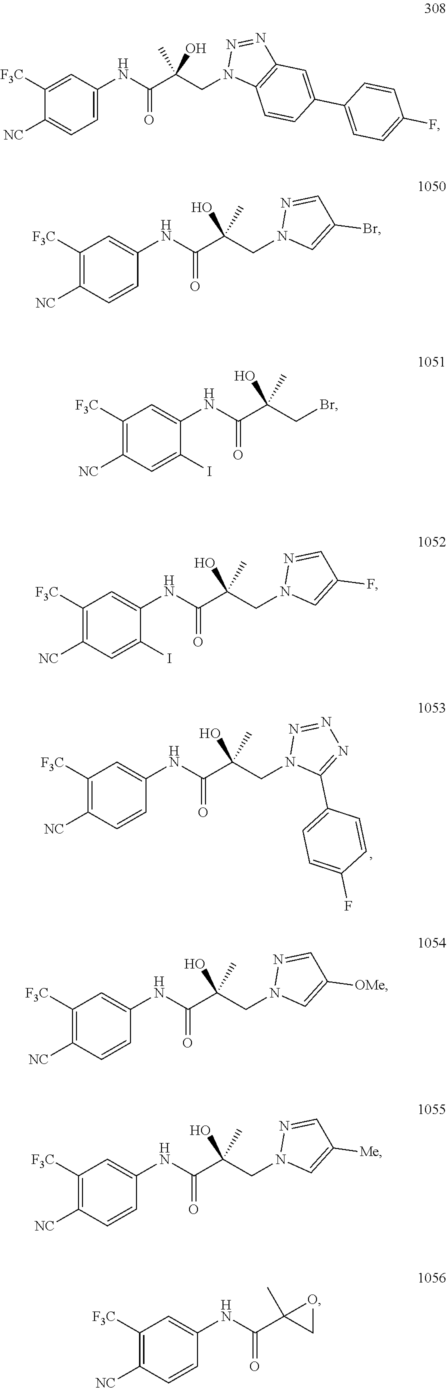

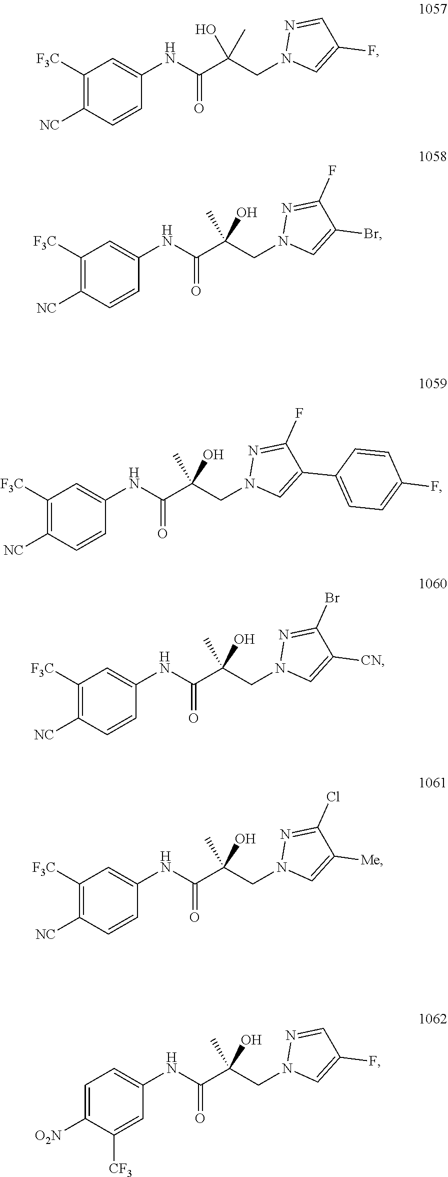 [SMILES: CC(O)(Cn1cc(F)cn1)C(=O)Nc1ccc(C#N)c(C(F)(F)F)c1 ; C[C@](O)(Cn1cc(Br)c(F)n1)C(=O)Nc1ccc(C#N)c(C(F)(F)F)c1 ; C[C@](O)(Cn1cc(-c2ccc(F)cc2)c(F)n1)C(=O)Nc1ccc(C#N)c(C(F)(F)F)c1 ; C[C@](O)(Cn1cc(C#N)c(Br)n1)C(=O)Nc1ccc(C#N)c(C(F)(F)F)c1 ; Cc1cn(C[C@](C)(O)C(=O)Nc2ccc(C#N)c(C(F)(F)F)c2)nc1Cl ; C[C@](O)(Cn1cc(F)cn1)C(=O)Nc1ccc([N+](=O)[O-])c(C(F)(F)F)c1]

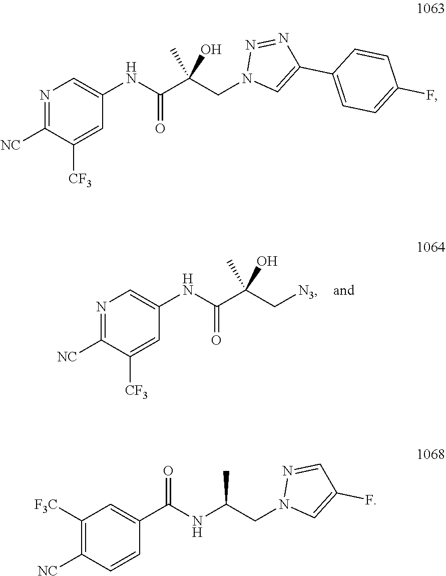 [SMILES: C[C@](O)(Cn1cc(-c2ccc(F)cc2)nn1)C(=O)Nc1cnc(C#N)c(C(F)(F)F)c1 ; C[C@](O)(CN=[N+]=[N-])C(=O)Nc1cnc(C#N)c(C(F)(F)F)c1 ; "and" ; C[C@@H](Cn1cc(F)cn1)NC(=O)c1ccc(C#N)c(C(F)(F)F)c1]

One embodiment of the invention encompasses the SARD compound having at least one of the following properties: binds to the AR through an alternate binding and degradation domain (BDD), e.g. in the NTD; binds to the AR through the AR ligand binding domain (LBD); exhibits AR-splice variant (AR-SV) degradation activity; exhibits AR-full length (AR-FL) degradation activity including pathogenic mutations thereof; exhibits AR-SV inhibitory activity (i.e., is an AR-SV antagonist); exhibits AR-FL inhibitory activity (i.e., is an AR-FL antagonist) including pathogenic mutations thereof; possesses dual AR-SV degradation and AR-SV inhibitory functions; and/or dual AR-FL degradation and AR-FL inhibitory functions.

Another embodiment of the invention encompasses pharmaceutical compositions comprising a SARD compound according to this invention, or its isomer, pharmaceutically acceptable salt, pharmaceutical product, polymorph, hydrate or any combination thereof, and a pharmaceutically acceptable carrier. The pharmaceutical composition may be formulated for topical use. The topical pharmaceutical composition may be a solution, lotion, salve, cream, ointment, liposome, spray, gel, foam, roller stick, cleansing soaps or bars, emulsion, mousse, aerosol, or shampoo.

The invention encompasses a method of treating prostate cancer (PCa) or increasing survival in a male subject in need of treatment comprising administering to the subject a therapeutically effective amount of a compound defined by formulas I-IX, IA-ID, IIA, IIB, VIIA, VIIB, VIIIA, VIIIB, IXA or IXB or any of compounds disclosed herein. The invention encompasses a method of treating prostate cancer (PCa) or increasing survival in a male subject in need of treatment comprising administering to the subject a therapeutically effective amount of a compound defined by formulas 44-46, 98, 300-308, 1050-1064, and 1068. The prostate cancer includes, but is not limited to, advanced prostate cancer, castration resistant prostate cancer (CRPC), metastatic CRPC (mCRPC), non-metastatic CRPC (nmCRPC), high-risk nmCRPC or any combination thereof. Another embodiment of the invention encompasses the method further comprising administering androgen deprivation therapy. Alternatively, the method may treat a prostate or other cancer that is resistant to treatment with known androgen receptor antagonist(s) or ADT. In another embodiment, the method may treat darolutamide resistant prostate cancer. In another embodiment, the method may treat enzalutamide resistant prostate cancer. In another embodiment, the method may treat apalutamide resistant prostate cancer. In another embodiment, the method may treat abiraterone resistant prostate cancer. Yet another embodiment of the invention encompasses a method of treating prostate or other AR antagonist resistant cancer with a SARD compound of the invention wherein the androgen receptor antagonist(s) is at least one of enzalutamide, apalutamide, bicalutamide, abiraterone, ODM-201 (darolutamide), EPI-001, EPI-506, AZD-3514, galeterone, ASC-J9, flutamide, hydroxyflutamide, nilutamide, cyproterone acetate, ketoconazole, or spironolactone.

In some embodiments, the prostate cancer is AR antagonist resistant prostate cancer which overexpresses the glucocorticoid receptor (GR). In some embodiments, activation of the GR provides support for growth of the prostate cancer and/or confers antiandrogen resistance to the prostate cancer. In some embodiments, SARDs of this invention can be used to treat GR-dependent or GR-overexpressing prostate cancers, whether antiandrogen resistant or not.

Yet another embodiment of the invention encompasses a method of treating prostate or other cancers using a SARD compound of the invention wherein the other cancers are selected from breast cancer such as triple negative breast cancer (TNBC), testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer. In another embodiment, the breast cancer is triple negative breast cancer (TNBC).

The invention encompasses a method of reducing the levels of AR-splice variants in a subject comprising administering to the subject a therapeutically effective amount of a compound of this invention, or its isomer, pharmaceutically acceptable salt, pharmaceutical product, polymorph, hydrate or any combination thereof. The method may comprise further reducing the levels of AR-full length in the subject.

Another embodiment of the invention encompasses a method of treating Kennedy's disease in a subject comprising administering to the subject a compound of formulas I-IX, IA-ID, IIA, IIB, VIIA, VIIB, VIIIA, VIIIB, IXA or IXB or a compound of another formula of the invention.

Another embodiment of the invention encompasses a method of treating Kennedy's disease in a subject comprising administering to the subject a compound of formulas 44-46, 98, 300-308, 1050-1064, and 1068.

Yet another embodiment of the invention encompasses a method of: (a) treating acne in a subject, e.g., acne vulgaris; (b) decreasing sebum production in a subject, e.g., treats sehorrhea, seborrheic dermatitis, or acne; (c) treating hirsutism in a subject, e.g., female facial hair; (d) treating alopecia in a subject, e.g., androgenic alopecia, alopecia areata, alopecia secondary to chemotherapy, alopecia secondary to radiation therapy, alopecia induced by scarring, or alopecia induced by stress; (e) treating a hormonal condition in female, e.g., precocious puberty, early puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, or vaginal dryness; (f) treating sexual perversion, hypersexuality, or paraphilias in a subject; (g) treating androgen psychosis in a subject; (h) treating virilization in a subject; (i) treating complete or partial androgen insensitivity syndrome in a subject; (j) increasing or modulating ovulation in an animal; (k) treating of cancer in a subject; or any combination thereof, by administering a compound of this invention or a pharmaceutical composition thereof.

One embodiment of the invention encompasses methods of reducing the levels of polyglutamine (polyQ) AR polymorphs in a subject comprising administering a compound according to this invention. The method may inhibit, degrade, or both the function of the polyglutamine (polyQ) AR polymorphs (polyQ-AR). The polyQ-AR may be a short polyQ polymorph or a long polyQ polymorph. When the polyQ-AR is a short polyQ polymorph, the method further treats dermal disease. When the polyQ-AR is a long polyQ polymorph, the method further treats Kennedy's disease.

Another embodiment of the invention encompasses methods of treating amyotrophic lateral sclerosis (ALS) in a subject by administering a therapeutically effective amount of the compound of the invention, or its isomer, pharmaceutically acceptable salt, pharmaceutical product, polymorph, hydrate or any combination thereof; or a pharmaceutical composition thereof.

Another embodiment of the invention encompasses methods of treating abdominal aortic aneurysm (AAA) in a subject by administering a therapeutically effective amount of the compound of the invention, or its isomer, pharmaceutically acceptable salt, pharmaceutical product, polymorph, hydrate or any combination thereof; or a pharmaceutical composition thereof.

Yet another embodiment of the invention encompasses methods of treating uterine fibroids in a subject by administering a therapeutically effective amount of the compound of this invention, or its isomer, pharmaceutically acceptable salt, pharmaceutical product, polymorph, hydrate or any combination thereof; or a pharmaceutical composition thereof.

In yet another aspect, the invention provides a method of treating, suppressing, reducing the incidence, reducing the severity, or inhibiting the progression of a hormonal condition in a male in need thereof, comprising administering to the subject a therapeutically effective amount of a selective androgen receptor degrader (SARD) compound of the invention. In one embodiment, the condition in the method of the invention is hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers.

In one embodiment, the condition in the method of the invention is sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIGS. 1A-1C present in vitro AR antagonism of representative compounds. COS7 cells were transfected with 0.25 μg GRE-LUC, 0.01 g CMV-renilla LUC, and 25 ng CMV-hAR using Lipofectamine in OptiMEM® medium. Cells were treated 24 hours after transfection with a dose response at the indicated concentrations of the representative compounds, and luciferase assay performed 48 hours after transfection. Firefly luciferase values were normalized to renilla luciferase values. See Table 1 for compound structures.

FIGS. 2A and 2B present in vitro AR antagonism of representative compounds. See Table 1 for compound structures.

FIG. 3 presents FL AR degradation activity for representative compounds. The numbers under each lane represents the % change from vehicle. The bands were quantified using ImageJ® software. For each lane, the AR band was divided by GAPDH band and the % difference from vehicle was calculated and represented under each lane. The numbers shown are 0 (no degradation) or represented as decreases in AR levels normalized for GAPDH levels (some values represented as positive but still indicate degradation). From this experiment, it is apparent that 1048, 1058 and 1017 are high efficacy AR degraders at 3 μM dose. See Table 1 for compound structures.

FIG. 4 depicts Hershberger assay results to study the body weight changes of representative compounds. Intact Sprague Dawley rats (100-120 g body weight) (n=6/group) were dosed at 20 mg/kg daily for 13 days. Dosing solutions were prepared in 20% DMSO+80% PEG. Fourteen days after the initiation of treatment, animals were sacrificed and tissue weights were recorded. Body weights were measured on day 1 and at the time of sacrifice. Tissue weights were normalized to body weight and represented as percent change from vehicle-treated animals. As can be seen, most of these compounds did not significantly decreased body weight, suggesting that there is no gross toxicity for these compounds at this dose. See Table 1 for compound structures.

FIGS. 5A-5B present seminal vesicles changes following 20 mg/kg daily dosing. Each of the tested compounds exerted at least some AR antagonism in vivo as revealed in decreased support of the seminal vesicles organ weight. Note that even though indole 11 is the most potent SARD in vitro (IC₅₀ of 85.1 nM in Table 1 and 29 nM in Table 4), 11 was the weakest AR antagonist in vivo for this set of compounds, demonstrating less than 20% change from vehicle treated rats. Triazole 1045, pyrazoles 1017 and 1002 (Tart) performed equivocally to the pyrazole 1002 in this assay, whereas 1022 and 1058 may have exhibited slightly more efficacy at this dose. (Example 8) However, none of the compounds reduced seminal vesicles weight as much as castration, suggesting that higher doses and/or better bioavailability would be desirable. See Table 1 for compound structures.

FIG. 6 presents in vivo AR antagonism of 1048, 1065, 1058, 1022, and 1002 with regard to seminal vesicles weight reduction and corresponding serum concentrations and in vitro antagonism and degradation. 5 mg/kg dose for 1048 is shown in this figure. All other compounds are dosed at 20 mg/kg in this figure. Each of the four compounds produced in vivo antagonism in excess of 1002 which, assuming similar bioavailabilities, agrees well with 3-4 fold increased in vitro antagonism and improved degradation (Table 4). At sacrifice, blood samples were taken and serum drug levels were determined. These levels revealed that 1065, 1058, and 1022 had approximately 17-, 12- and 2-fold higher serum levels in rats when dosed at 20 mg/kg (Table 4). Correspondingly, 1065 demonstrated unexpectedly improved oral bioavailability compared to all previous SARD compounds, demonstrating full AR antagonism in vivo. See Table 1 for compound structures [Example 9].

FIGS. 7A-7D present % difference in organ weight from 1002 (% Diff from 1002) with 1002 defined as 0% change and vehicle defined as 100% change. When prostate and seminal vesicles weight reductions for all the compounds over the two studies are reported together, several compounds produced comparable to slightly improved efficacies compared to 1002. However, 1065 reached castration levels for seminal vesicles. See Table 1 for compound structures.

FIG. 8 presents serum testosterone levels of representative compounds. For animals treated with the 20 mg/kg of the indicated compounds, blood was drawn at the time of sacrifice and serum isolated. The serum was run through a LC-MS/MS to detect testosterone levels. As can be seen, even at levels much higher than those that produced chemical castration for 1065, there is no significant reduction in serum testosterone levels. Similar results were obtained for 1002 and 1058. This indicated that SARDs do not have any effect of the synthesis of testosterone but are potent in vivo AR antagonists by virtue of direct effects on AR. Further, this highlights that SARDs are potent antagonists which are capable of overcoming the endogenous androgens present in these intact animals. mpk-mg/kg. See Table 1 for compound structures.

FIGS. 9A-9B present GR antagonism of 1058 and dexamethasone as positive control. 1058 is a potent AR antagonist in vitro (83.7 nM) and capable of SV and FL AR degradation (70 and 80%, respectively). Further, dose response of 1058 in this GR transactivation assay in antagonist mode produced potent (1984 nM) and complete (comparable efficacy to RU486) GR antagonism in vitro. As a representive example, the lack of GR antagonism for 1002 is shown in FIG. 9B. As a positive control, dexamethasone demonstrated potent and high efficacy agonism in the same assay system (FIG. 9A). This, combined with the unexpected bioavailability, suggest that 1058 may be able to overcome or prevent the emergence of antiandrogen resistance mediated by GR, as discussed in Horm Cancer. (2014) 5(2), 72-89 or doi:10.1007/s12672-014-0173-2 and Cell (2013) 155, 1309-1322 or doi: 10.1016/j.cell.2013.11.012. See Table 1 for compound structures.

FIGS. 10A-OB present PR antagonism of 1058 and 1002. 1058, like many of the SARDs (1002 is shown), is also a potent PR antagonist (144 nM) in vitro suggesting the possibility of the treatment of breast cancers as well. Progesterone is a potent and high efficacy agonist in this system, providing a positive control for this assay.

FIGS. 11A-11D present improved pure antagonism and SARD activity of 1061, 1068, and 1002 (see Example 2 and Table 1 for structures).

FIG. 12 presents degradation of AR and AR-V7 with 1058, 1002, and 1065 in LNCaP-ARV7 cells (see Example 12 for structures). LNCaP-ARV7 cells were treated in growth medium for 24 hours. Cells were harvested, protein extracted, and Western blot for AR and GAPDH was performed. 1058 is more potent in degrading the AR and AR-V7. Expression of AR-V7 was induced by doxycycline addition to LNCaP-ARV7. Additionally, 1058, 1002, and 1065 caused the degradation of AR full length (which is a T877A mutant) and AR-V7. Results demonstrated that 1058 is more active at degrading the AR-V7 than close structural analog 1002. 1058 nearly completely degraded both at 10 M.

FIG. 13 presents the inhibition of R1881-dependent FKBP5 gene expression in LNCaP-ARV7 cells by 1002 and 1058. LNCaP-ARV7 cells maintained in charcoal-stripped serum containing medium were treated for 24 hours. RNA was extracted and expression of FKBP5 was measured and normalized to GAPDH using realtime PCR. 1058 is more potent than 1002. An unexpected 10-fold increased potency of 1058 was observed in the antagonism of R1881-induced expression of FKBP5, a classically known AR-dependent gene, in LNCaP-ARV7 cells.

FIG. 14 presents antiproliferative activities of 1002 and 1058 in LNCaP-ARV7 cells. LNCaP-ARV7 cells were plated in full serum and treated as indicated for 6 days (with medium change and retreatment after 3 days). Viable cells were measured using CellTiter Glo assay. 1058 inhibits the proliferation of cells starting from 0.3 uM. 1058 more potently inhibited AR-V7 dependent proliferation of LNCaP-ARV7 cells with antagonism seen of 0.3 M for 1058 vs. 1 M for 1002.

FIG. 15 presents inhibition of AR-V7 dependent FKBP5 gene expression in 22RV1 cells by 1002 and 1058. 22RV1 cells were treated in charcoal stripped serum containing medium for 3 days. RNA was isolated and expression of AR-target gene, FKBP5, was quantified and normalized to GAPDH using realtime PCR. 1058 inhibits even baseline activity in 22RV1 cells, which was mediated by AR-V7. 22RV1 prostate cancer cells endogeneously and constitutively express both full length AR (AR) and AR-V7. The bulk of the baseline AR-axis activity in 22RV1 cells is believed to be due to AR-V7 activity as reflected by poor AR antagonism of proliferation and AR-dependent genes by LBD-directed anti-androgens such as enzalutamide in 22RV1 (not shown). FIG. 15 shows that 1058 but not 1002 was able to inhibit the AR-V7 dependent baseline expression (i.e., in the absence of an added androgen to active AR-FL) of the AR-dependent gene FKBP5 in 22RV1 cells. Given the structural similarity of 1058 and 1002, it is unexpected that 1058 was >3-fold more potent than 1002 and 1002 did not demonstrate any efficacy in the dose range tested. The increased potency and efficacy of 1058 vs. 1002 shown in FIGS. 12-15 to degrade and inhibit the activity of AR-V7 in prostate cancer cells was unexpected and suggested improved ability to treat AR-SV dependent prostate cancers including presently untreatable CRPCs.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Androgens act in cells by binding to the AR, a member of the steroid receptor superfamily of transcription factors. As the growth and maintenance of prostate cancer (PCa) is largely controlled by circulating androgens, treatment of PCa heavily relies on therapies that target AR. Treatment with AR antagonists such as enzalutamide, apalutamide, bicalutamide or hydroxyflutamide to disrupt receptor activation has been successfully used in the past to reduce PCa growth. All currently available AR antagonists competitively bind AR and recruit corepressors such as NCoR and SMRT to repress transcription of target genes. However, altered intracellular signaling, AR mutations, and increased expression of coactivators lead to functional impairment of antagonists or even transformation of antagonists into agonists. Studies have demonstrated that mutation of W741 and T877 within AR converts bicalutamide and hydroxyflutamide, respectively, to agonists. Similarly, increased intracellular cytokines recruit coactivators instead of corepressors to AR-responsive promoters subsequently converting bicalutamide to an agonist. Similarly, mutations that have been linked to enzalutamide and apalutamide resistance include F876, H874, T877, and di-mutants T877/5888, T877/D890, F876/T877 (i.e., MR49 cells), and H874/T877 (Genome Biol. (2016) 17:10 (doi: 10.1186/s13059-015-0864-1)). Abiraterone resistance mutations include L702H mutations which results in activation of the AR by glucocorticoids such as prednisone, causing resistance to abiraterone because abiraterone is usually prescribed in combination with prednisone. If resistance develops to enzalutamide or apalutamide then often the patient is refractory to abiraterone also and vice versa; or the duration of response is very short. This situation highlights the need for a definitive androgen ablation therapy to prevent AR reactivation in advanced prostate cancers. Arora et al. in Cell 155, 1309-1322 reported the induction of glucocorticoid receptor (GR) expression as a common feature of drug-resistant tumors from prostate cancer cell lines (LNCaP/AR) and clinical samples. GR substituted for the AR to activate a similar but distinguishable set of target genes and was necessary for maintenance of the resistant phenotype. The GR agonist dexamethasone was sufficient to confer enzalutamide (or apalutamide) resistance, whereas a GR antagonist restored sensitivity. Acute AR inhibition resulted in GR upregulation in a subset of prostate cancer cells due to relief of AR-mediated feedback repression of GR expression. These findings establish a mechanism of escape from AR blockade through expansion of cells primed to drive AR target genes via an alternative nuclear receptor upon drug exposure. In some cases, the SARDs of this invention are potent GR antagonists in addition to potent AR antagonists. As such, they would possibly prevent the emergence of GR-dependent antiandrogen resistance or treat antiandrogen resistant prostate cancers which are dependent on GR.

Despite initial response to androgen deprivation therapy (ADT), PCa disease progression is inevitable and the cancer emerges as castration-resistant prostate cancer (CRPC). The primary reason for castration resistant prostate cancer (CRPC) re-emergence is re-activation of androgen receptor (AR) by alternate mechanisms such as:
(a) intracrine androgen synthesis;
(b) expression of AR splice variants (AR-SV), e.g., that lack ligand binding domain (LBD);
(c) AR-LBD mutations with potential to resist antagonists;
(d) hyper-sensitization of AR to low androgen levels, e.g., due to AR gene amplification or AR mutation;
(e) amplification of the AR gene within the tumor; and
(f) over expression of coactivators and/or altered intracellular signal transduction.

The invention encompasses novel selective androgen receptor degrader (SARD) compounds encompassed by 44-46, 98, 300-308, 1050-1064, and 1068, which inhibit the growth of prostate cancer (PCa) cells and tumors that are dependent on AR full length (AR-FL) including pathogenic and resistance mutations and wildtype, and/or AR splice variants (AR-SV) for proliferation.

The invention further encompasses novel selective androgen receptor degrader (SARD) compounds encompassed by formula I, which inhibit the growth of prostate cancer (PCa) cells and tumors that are dependent on AR full length (AR-FL) including pathogenic and resistance mutations and wildtype, and/or AR splice variants (AR-SV) for proliferation.

As used herein, unless otherwise defined, a “selective androgen receptor degrader” (SARD) compound is an androgen receptor antagonist capable of inhibiting the growth of PCa cells and tumors that are dependent on AR-full length (AR-FL) and/or AR splice variants (AR-SV) for proliferation. The SARD compound may not bind to ligand binding domain (LBD). Alternatively, a “selective androgen receptor degrader” (SARD) compound is an androgen receptor antagonist capable of causing degradation of a variety of pathogenic mutant variant AR's and wildtype AR and hence are capable of exerting anti-androgenism is a wide variety of pathogenic altered cellular environments found in the disease states embodied in this invention. In one embodiment, the SARD is orally active. In another embodiment, the SARD is applied topically to the site of action.

The SARD compound may bind to the N-terminal domain (NTD) of the AR; to an alternate binding and degradation domain (BDD) of the AR; to both the AR ligand binding domain (LBD) and to an alternate binding and degradation domain (BDD); or to both the N-terminal domain (NTD) and to the ligand binding domain (LBD) of the AR. In one embodiment, the BDD may be located in the NTD. In one embodiment, the BDD is located in the AF-1 region of the NTD. Alternatively, the SARD compound may be capable of: inhibiting growth driven by the N-terminal domain (NTD)-dependent constitutively active AR-SV; or inhibiting the AR through binding to a domain that is distinct from the AR LBD. Also, the SARD compound may be a strong (i.e., highly potent and highly efficacious) selective androgen receptor antagonist, which antagonizes the AR stronger than other known AR antagonists (e.g., enzalutamide, apalutamide, bicalutamide and abiraterone).

The SARD compound may be a selective androgen receptor antagonist, which targets AR-SVs, which cannot be inhibited by conventional antagonists. The SARD compound may exhibit any one of several activities including, but not limited to: AR-SV degradation activity; AR-FL degradation activity; AR-SV inhibitory activity (i.e., is an AR-SV antagonist); AR-FL inhibitory activity (i.e., is an AR-FL antagonist); inhibition of the constitutive activation of AR-SVs; or inhibition of the constitutive activation of AR-FLs. Alternatively, the SARD compound may possess dual AR-SV degradation and AR-SV inhibitory functions, and/or dual AR-FL degradation and AR-FL inhibitory functions; or alternatively possess all four of these activities.

The SARD compound may also degrade AR-FL and AR-SV. The SARD compound may degrade the AR through binding to a domain that is distinct from the AR LBD. The SARD compound may possess dual degradation and AR-SV inhibitory functions that are distinct from any available CRPC therapeutics. The SARD compound may inhibit the re-activation of the AR by alternate mechanisms such as: intracrine androgen synthesis, expression of AR-SV that lack ligand binding domain (LBD) and AR-LBD mutations with potential to resist antagonists, or inhibit re-activated androgen receptors present in pathogenic altered cellular environments.

Examples of AR-splice variants include, but are not limited to, AR-V7 and ARv567es (a.k.a. AR-V12; S. Sun, et al. Castration resistance in human prostate cancer is conferred by a frequently occurring androgen receptor splice variant. J Clin Invest. (2010) 120(8), 2715-2730). Nonlimiting examples of AR mutations conferring antiandrogen resistance are: W741L, T877A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029) mutations. Many other LBD resistance conferring mutations are known in the art and will continue to be discovered. AR-V7 is a splice variant of AR that lacks the LBD (A. H. Bryce & E. S. Antonarakis. Androgen receptor splice variant 7 in castration-resistant prostate cancer: Clinical considerations. Int J Urol. (2016 Jun. 3) 23(8), 646-53. doi: 10.1111/iju.13134). It is constitutively active and has been demonstrated to be responsible for aggressive PCa and resistance to endocrine therapy.

The invention encompasses novel selective androgen receptor degrader (SARD) compounds of formulas 44-46, 98, 300-308, 1050-1064, and 1068 which bind to the AR through an alternate binding and degradation domain (BDD), e.g., the NTD or AF-1. The SARDs may further bind the AR ligand binding domain (LBD).

The invention further encompasses novel selective androgen receptor degrader (SARD) compounds of formulas I-IX, IA-ID, IIA, IIB, VIIA, VIIB, VIIIA, VIIIB, IXA or IXB which bind to the AR through an alternate binding and degradation domain (BDD), e.g., the NTD or AF-1. The SARDs may further bind the AR ligand binding domain (LBD).

The SARD compounds may be used in treating CRPC that cannot be treated with any other antagonist. The SARD compounds may treat CRPC by degrading AR-SVs. The SARD compounds may maintain their antagonistic activity in AR mutants that normally convert AR antagonists to agonists. For instance, the SARD compounds maintain their antagonistic activity to AR mutants W741L, T877A, and F876L (J. D. Joseph et al. A clinically relevant androgen receptor mutation confers resistance to second-generation antiandrogens enzalutamide and ARN-509 [apalutamide]. Cancer Discov. (2013) 3(9), 1020-1029). Alternatively, the SARD compounds elicit antagonistic activity within an altered cellular environment in which LBD-targeted agents are not effective or in which NTD-dependent AR activity is constitutively active. Alternatively, SARD compounds can be co-antagonists of AR and GR and thereby overcome or prevent antiandrogen resistant CRPC in which GR is overexpressed and/or GR is activating the AR axis.

Selective Androgen Receptor Degrader (SARD) Compounds

The invention encompasses selective androgen receptor degrader (SARD) compounds selected from any one of the following structures:

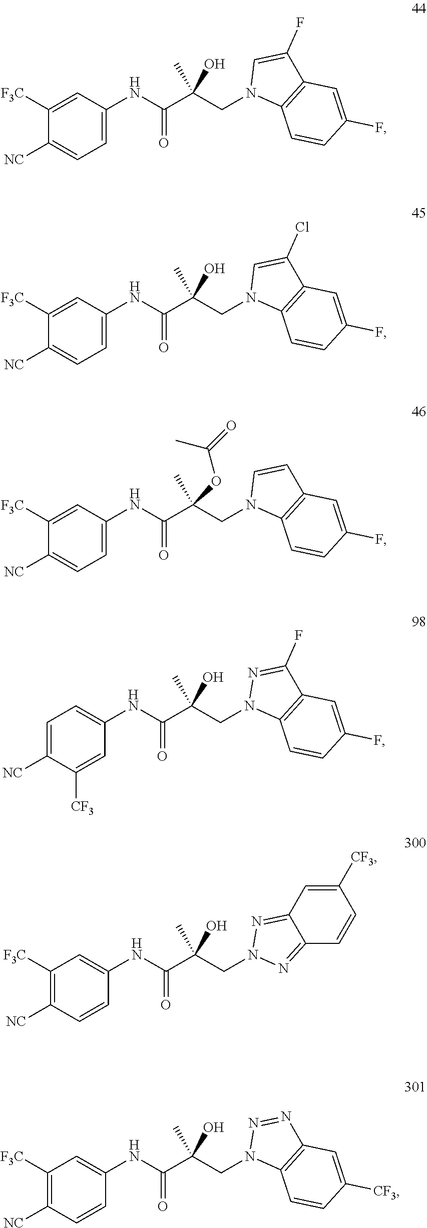 [SMILES: C[C@](O)(Cn1cc(F)c2cc(F)ccc21)C(=O)Nc1ccc(C#N)c(C(F)(F)F)c1 ; C[C@](O)(Cn1cc(Cl)c2cc(F)ccc21)C(=O)Nc1ccc(C#N)c(C(F)(F)F)c1 ; CC(=O)O[C@@](C)(Cn1ccc2cc(F)ccc21)C(=O)Nc1ccc(C#N)c(C(F)(F)F)c1 ; C[C@](O)(Cn1nc(F)c2cc(F)ccc21)C(=O)Nc1ccc(C#N)c(C(F)(F)F)c1 ; C[C@](O)(Cn1nc2ccc(C(F)(F)F)cc2n1)C(=O)Nc1ccc(C#N)c(C(F)(F)F)c1 ; C[C@](O)(Cn1nnc2cc(C(F)(F)F)ccc21)C(=O)Nc1ccc(C#N)c(C(F)(F)F)c1]

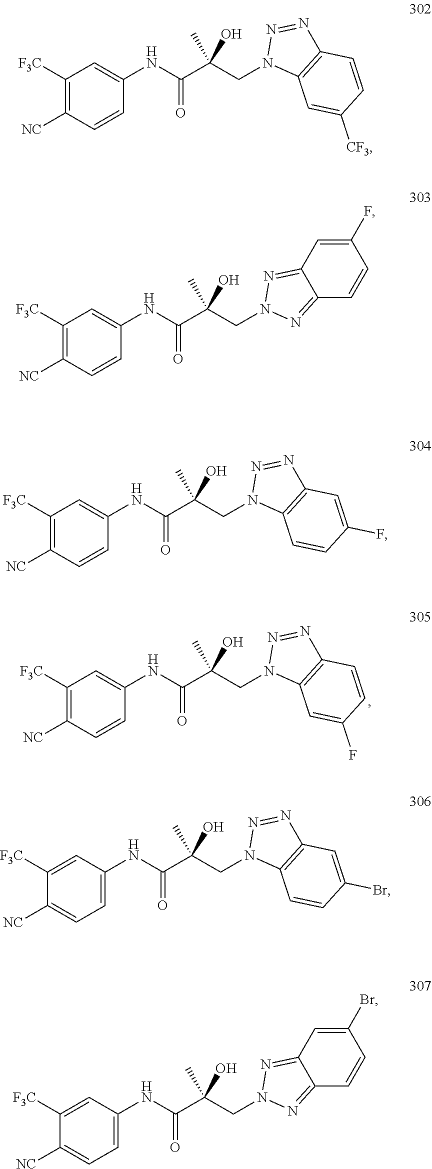 [SMILES: C[C@](O)(Cn1nnc2ccc(C(F)(F)F)cc21)C(=O)Nc1ccc(C#N)c(C(F)(F)F)c1 ; C[C@](O)(Cn1nc2ccc(F)cc2n1)C(=O)Nc1ccc(C#N)c(C(F)(F)F)c1 ; C[C@](O)(Cn1nnc2cc(F)ccc21)C(=O)Nc1ccc(C#N)c(C(F)(F)F)c1 ; C[C@](O)(Cn1nnc2ccc(F)cc21)C(=O)Nc1ccc(C#N)c(C(F)(F)F)c1 ; C[C@](O)(Cn1nnc2cc(Br)ccc21)C(=O)Nc1ccc(C#N)c(C(F)(F)F)c1 ; C[C@](O)(Cn1nc2ccc(Br)cc2n1)C(=O)Nc1ccc(C#N)c(C(F)(F)F)c1]

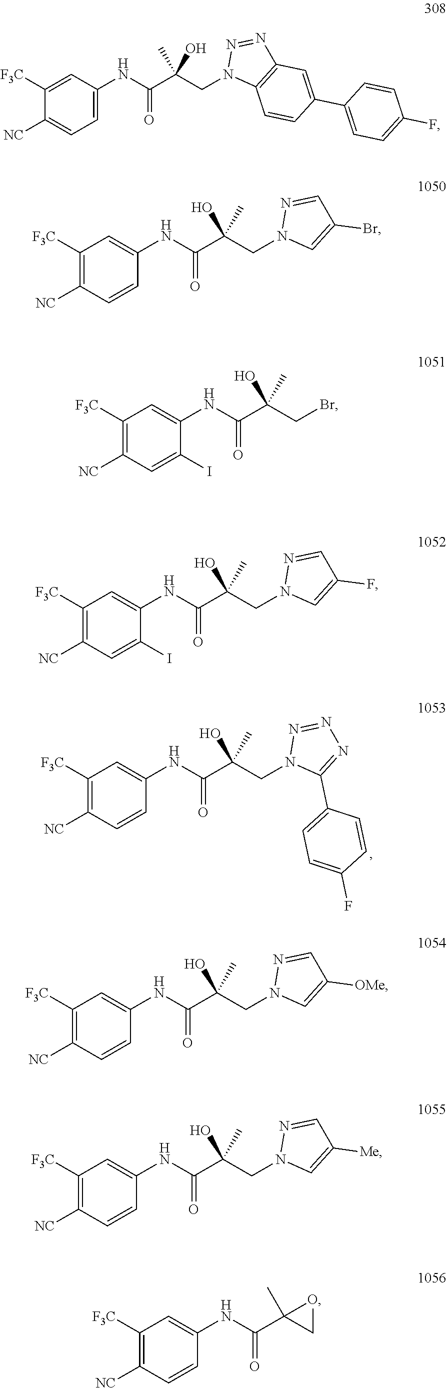

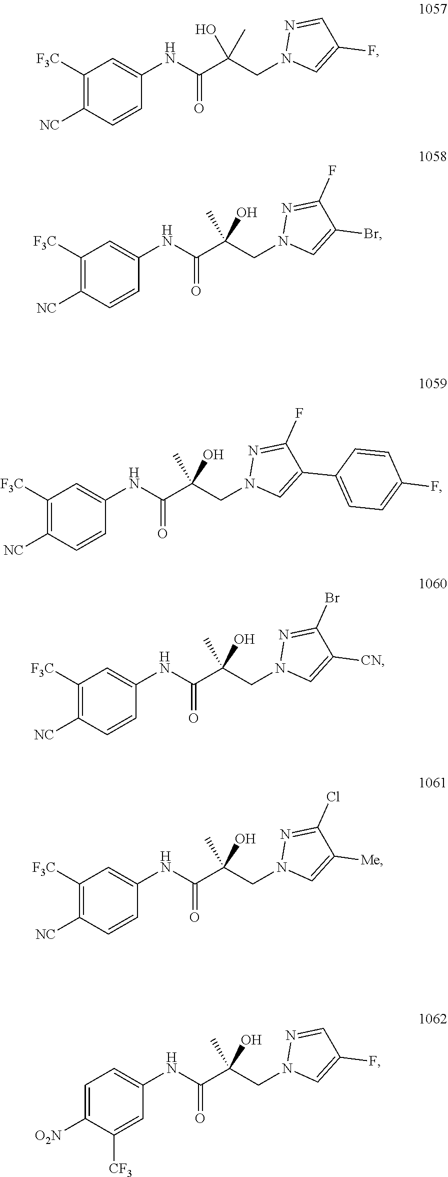 [SMILES: CC(O)(Cn1cc(F)cn1)C(=O)Nc1ccc(C#N)c(C(F)(F)F)c1 ; C[C@](O)(Cn1cc(Br)c(F)n1)C(=O)Nc1ccc(C#N)c(C(F)(F)F)c1 ; C[C@](O)(Cn1cc(-c2ccc(F)cc2)c(F)n1)C(=O)Nc1ccc(C#N)c(C(F)(F)F)c1 ; C[C@](O)(Cn1cc(C#N)c(Br)n1)C(=O)Nc1ccc(C#N)c(C(F)(F)F)c1 ; Cc1cn(C[C@](C)(O)C(=O)Nc2ccc(C#N)c(C(F)(F)F)c2)nc1Cl ; C[C@](O)(Cn1cc(F)cn1)C(=O)Nc1ccc([N+](=O)[O-])c(C(F)(F)F)c1]

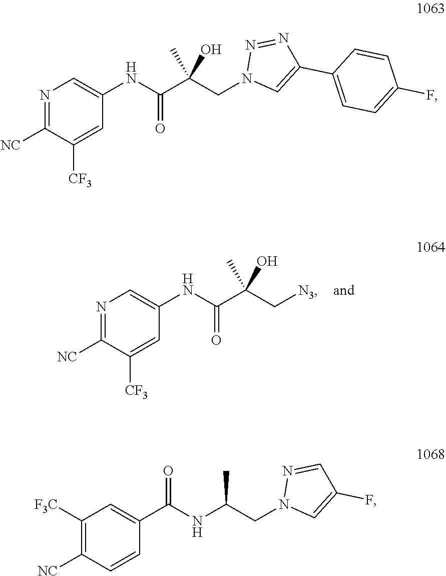 [SMILES: C[C@](O)(Cn1cc(-c2ccc(F)cc2)nn1)C(=O)Nc1cnc(C#N)c(C(F)(F)F)c1 ; C[C@](O)(CN=[N+]=[N-])C(=O)Nc1cnc(C#N)c(C(F)(F)F)c1 ; "and" ; C[C@@H](Cn1cc(F)cn1)NC(=O)c1ccc(C#N)c(C(F)(F)F)c1]

or its optical isomer, isomer, pharmaceutically acceptable salt, pharmaceutical product, polymorph, hydrate or any combination thereof.

The invention encompasses selective androgen receptor degrader (SARD) compounds represented by the structure of formula I:

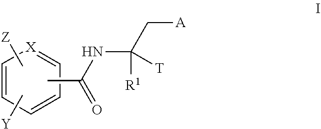

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R3;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its optical isomer, isomer, pharmaceutically acceptable salt, pharmaceutical product, polymorph, hydrate or any combination thereof.

In various embodiments, the SARD compound of formula I has a chiral carbon. In other embodiments, the SARD compound of formula I is a racemic mixture. In other embodiments, the SARD compound of formula I is an (S) isomer. In other embodiments, the SARD compound of formula I is an (R) isomer.

The invention encompasses selective androgen receptor degrader (SARD) compounds represented by the structure of formula IA:

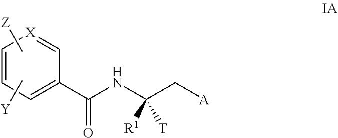

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its isomer, pharmaceutically acceptable salt, pharmaceutical product, polymorph, hydrate or any combination thereof.

The invention encompasses selective androgen receptor degrader (SARD) compounds represented by the structure of formula IB:

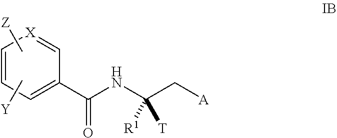

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its isomer, pharmaceutically acceptable salt, pharmaceutical product, polymorph, hydrate or any combination thereof.

The invention encompasses selective androgen receptor degrader (SARD) compounds represented by the structure of formula IC:

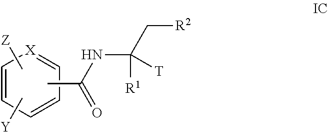

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its optical isomer, isomer, pharmaceutically acceptable salt, pharmaceutical product, polymorph, hydrate or any combination thereof.

The invention encompasses selective androgen receptor degrader (SARD) compounds represented by the structure of formula ID:

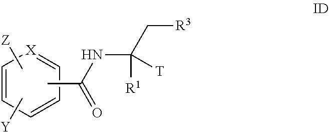

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴H, is alkyl, haloalkyl, cycloalkyl, aryl or heteroaryl, wherein said alkyl, haloalkyl, cycloalkyl, aryl or heteroaryl groups are optionally substituted;

or its optical isomer, isomer, pharmaceutically acceptable salt, pharmaceutical product, polymorph, hydrate or any combination thereof.

The invention encompasses a SARD compound represented by the structure of formula II:

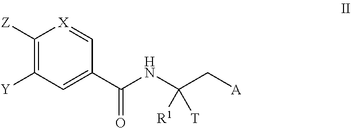

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its optical isomer, isomer, pharmaceutically acceptable salt, pharmaceutical product, polymorph, hydrate or any combination thereof.

In various embodiments, the SARD compound of formula II has a chiral carbon. In other embodiments, the SARD compound of formula II is a racemic mixture. In other embodiments, the SARD compound of formula II is an (S) isomer. In other embodiments, the SARD compound of formula II is an (R) isomer.

The invention encompasses a SARD compound represented by the structure of formula IIA:

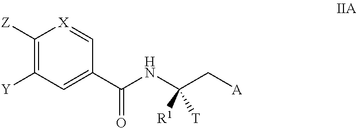

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;
or T and R¹ form a 3-8 carbocyclic or heterocyclic ring;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocylic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its isomer, pharmaceutically acceptable salt, pharmaceutical product, polymorph, hydrate or any combination thereof.

The invention encompasses a SARD compound represented by the structure of formula IIB:

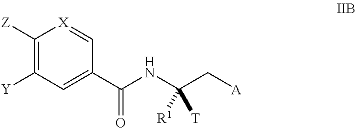

wherein

R¹ is H, CH₃, CH₂F, CHF₂, CF₃, CH₂CH₃, or CF₂CF₃;
or T and R¹ form a 3-8 carbocyclic or heterocyclic ring;

T is H, OH, OR, OCOR, CH₃, —NHCOCH₃, or NHCOR;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its isomer, pharmaceutically acceptable salt, pharmaceutical product, polymorph, hydrate or any combination thereof.

The invention encompasses a SARD compound represented by the structure of formula III:

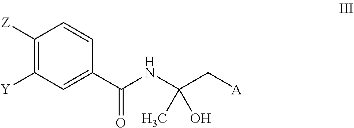

wherein

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its optical isomer, isomer, pharmaceutically acceptable salt, pharmaceutical product, polymorph, hydrate or any combination thereof.

In various embodiments, the SARD compound of formula III has a chiral carbon. In other embodiments, the SARD compound of formula III is a racemic mixture. In other embodiments, the SARD compound of formula III is an (S) isomer. In other embodiments, the SARD compound of formula III is an (R) isomer.

The invention encompasses a selective androgen receptor degrader compound represented by the structure of formula IV:

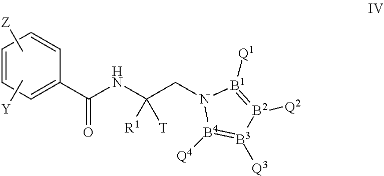

wherein

B¹, B², B³, and B⁴ are each independently carbon or nitrogen;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q¹, Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR; wherein if B¹, B², B³, or B⁴ is nitrogen then Q¹, Q², Q³, or Q⁴, respectively, is nothing;

or its optical isomer, isomer, pharmaceutically acceptable salt, pharmaceutical product, polymorph, hydrate or any combination thereof.

In various embodiments, the SARD compound of formula IV has a chiral carbon. In other embodiments, the SARD compound of formula IV is a racemic mixture. In other embodiments, the SARD compound of formula IV is an (S) isomer. In other embodiments, the SARD compound of formula IV is an (R) isomer.

The invention encompasses a selective androgen receptor degrader compound represented by the structure of formula V:

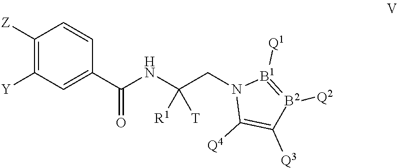

wherein

B¹ and B² are each independently carbon or nitrogen;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q¹, Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR; wherein if B¹ or B² is nitrogen then Q¹ or Q², respectively, is nothing;

or its optical isomer, isomer, pharmaceutically acceptable salt, pharmaceutical product, polymorph, hydrate or any combination thereof.

In various embodiments, the SARD compound of formula V has a chiral carbon. In other embodiments, the SARD compound of formula V is a racemic mixture. In other embodiments, the SARD compound of formula V is an (S) isomer. In other embodiments, the SARD compound of formula V is an (R) isomer.

The invention encompasses a selective androgen receptor degrader compound represented by the structure of formula VI:

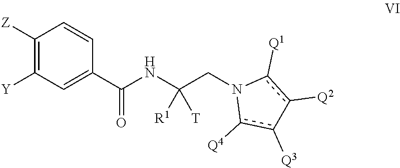

wherein

[symbol] is a single or double bond;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q¹, Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its optical isomer, isomer, pharmaceutically acceptable salt, pharmaceutical product, polymorph, hydrate or any combination thereof.

In various embodiments, the SARD compound of formula VI has a chiral carbon. In other embodiments, the SARD compound of formula VI is a racemic mixture. In other embodiments, the SARD compound of formula VI is an (S) isomer. In other embodiments, the SARD compound of formula VI is an (R) isomer.

The invention encompasses a selective androgen receptor degrader compound represented by the structure of formula VII:

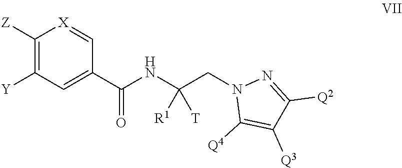

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its optical isomer, isomer, pharmaceutically acceptable salt, pharmaceutical product, polymorph, hydrate or any combination thereof.

In various embodiments, the SARD compound of formula VII has a chiral carbon. In other embodiments, the SARD compound of formula VII is a racemic mixture. In other embodiments, the SARD compound of formula VII is an (S) isomer. In other embodiments, the SARD compound of formula VII is an (R) isomer.

The invention encompasses a selective androgen receptor degrader compound represented by the structure of formula VIIA:

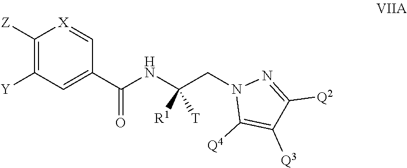

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its isomer, pharmaceutically acceptable salt, pharmaceutical product, polymorph, hydrate or any combination thereof.

The invention encompasses a selective androgen receptor degrader compound represented by the structure of formula VIIB:

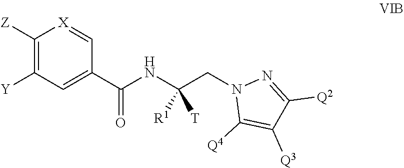

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q², Q³, or Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its isomer, pharmaceutically acceptable salt, pharmaceutical product, polymorph, hydrate or any combination thereof.

In another embodiment, the invention encompasses a selective androgen receptor degrader compound represented by the structure of formula VIII:

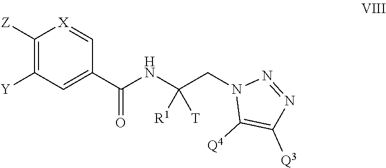

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q³ and Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its optical isomer, isomer, pharmaceutically acceptable salt, pharmaceutical product, polymorph, hydrate or any combination thereof.

In another embodiment, the invention encompasses a selective androgen receptor degrader compound represented by the structure of formula VIIIA:

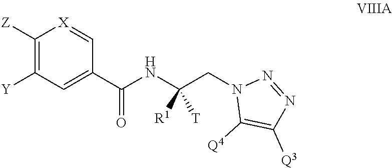

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q³ and Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its isomer, pharmaceutically acceptable salt, pharmaceutical product, polymorph, hydrate or any combination thereof.

In another embodiment, the invention encompasses a selective androgen receptor degrader compound represented by the structure of formula VIIIB:

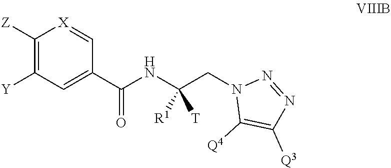

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH;
and Q³ and Q⁴ are each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;
   or its isomer, pharmaceutically acceptable salt, pharmaceutical product, polymorph, hydrate or any combination thereof.

In another embodiment, the invention encompasses a selective androgen receptor degrader compound represented by the structure of formula IX:

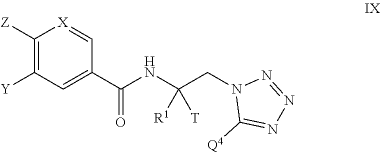

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q⁴ is selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its optical isomer, isomer, pharmaceutically acceptable salt, pharmaceutical product, polymorph, hydrate or any combination thereof.

In another embodiment, the invention encompasses a selective androgen receptor degrader compound represented by the structure of formula IXA:

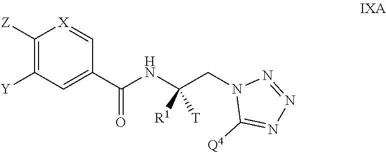

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH; and

Q⁴ is selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its isomer, pharmaceutically acceptable salt, pharmaceutical product, polymorph, hydrate or any combination thereof.

In another embodiment, the invention encompasses a selective androgen receptor degrader compound represented by the structure of formula IXB:

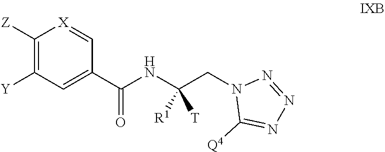

wherein

X is CH or N;

Y is H, CF₃, F, I, Br, Cl, CN, or C(R)₃;

Z is H, NO₂, CN, halide, COOH, COR, NHCOR, CONHR,

or Y and Z form a 5 to 8 membered fused ring;

R¹ is H, CH₃, CH₂F, CHF₂, CF₃, CH₂CH₃, or CF₂CF₃;

T is H, OH, OR, OCOR, CH₃, —NHCOCH₃, or NHCOR;
or T and R¹ form a 3-8 carbocyclic or heterocyclic ring;

R is H, alkyl, alkenyl, haloalkyl, alcohol, CH₂CH₂OH, CF₃, CH₂Cl, CH₂CH₂Cl, aryl, F, Cl, Br, I, or OH;

and

Q⁴ is selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

or its isomer, pharmaceutically acceptable salt, pharmaceutical product, polymorph, hydrate or any combination thereof.

In one embodiment, A of formula I-III, IA, IB, IIA, and IIB and R² of formula IC is a five or six-membered saturated or unsaturated ring having at least one nitrogen atom. In another embodiment, A is a substituted or unsubstituted pyrrole, pyrroline, pyrrolidine, pyrazole, pyrazoline, pyrazolidine, imidazole, imidazoline, imidazolidine, triazole, tetrazole, pyridine, morpholine, or other heterocyclic ring. Each represents a separate embodiment of this invention. In another embodiment, A is a five or six-membered heterocyclic ring. In another embodiment, a nitrogen atom of the five or six membered saturated or unsaturated ring is attached to the backbone structure of the molecule. In another embodiment, a carbon atom of the five or six membered saturated or unsaturated ring is attached to the backbone structure of the molecule.

In one embodiment, A of formula I-III, IA, IB, IIA, and IIB and R³ of formula ID is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), NO₂, cyanate, isocyanate, thiocyanate, isothiocyanate, mesylate, tosylate, triflate, PO(OH)₂ or OPO(OH)₂; wherein R⁴ is H, alkyl, haloalkyl, cycloalkyl, aryl or heteroaryl, wherein said alkyl, haloalkyl, cycloalkyl, aryl or heteroaryl groups are optionally substituted.

In one embodiment, A of formula I-III, IA, IB, IIA, and IIB and R³ of formula ID is NHR². In one embodiment, A of formula I-III, IA, IB, IIA, and IIB and R³ of formula ID is halide. In one embodiment, A of formula I-III, IA, IB, IIA, and IIB and R³ of formula ID is F. In one embodiment, A of formula I-III, IA, IB, IIA, and IIB and R³ of formula ID is Br. In one embodiment, A of formula I-III, IA, IB, IIA, and IIB and R³ of formula ID is Cl. In one embodiment, A of formula I-III, IA, IB, IIA, and IIB and R³ of formula ID is I. In one embodiment, A of formula I-III, IA, IB, IIA, and IIB and R³ of formula ID is N₃. In one embodiment, A of formula I-III, IA, IB, IIA, and IIB and R³ of formula ID is OR⁴. In one embodiment, A of formula I-III, IA, IB, IIA, and IIB and R³ of formula ID is CF₃. In one embodiment, A of formula I-III, IA, IB, IIA, and IIB and R³ of formula ID is COR⁴. In one embodiment, A of formula I-III, IA, IB, IIA, and IIB and R³ of formula ID is COCl. In one embodiment, A of formula I-III, IA, IB, IIA, and IIB and R³ of formula ID is COOCOR⁴. In one embodiment, A of formula I-III, IA, IB, IIA, and IIB and R³ of formula ID is COOR⁴. In one embodiment, A of formula I-III, IA, IB, IIA, and IIB and R³ of formula ID is OCOR⁴. In one embodiment, A of formula I-III, IA, IB, IIA, and IIB and R³ of formula ID is OCONHR⁴. In one embodiment, A of formula I-III, IA, IB, IIA, and IIB and R³ of formula ID is NHCOOR⁴. In one embodiment, A of formula I-III, IA, IB, IIA, and IIB and R³ of formula ID is NHCONHR⁴. In one embodiment, A of formula I-III, IA, IB, IIA, and IIB and R³ of formula ID is OCOOR⁴. In one embodiment, A of formula I-III, IA, IB, IIA, and IIB and R³ of formula ID is CN. In one embodiment, A of formula I-III, IA, IB, IIA, and IIB and R³ of formula ID is CON(R⁴)₂. In one embodiment, A of formula I-III, IA, IB, IIA, and IIB and R³ of formula ID is SR⁴. In one embodiment, A of formula I-III, IA, IB, IIA, and IIB and R³ of formula ID is SO₂R⁴. In one embodiment, A of formula I-III, IA, IB, IIA, and IIB and R³ of formula ID is SOR⁴. In one embodiment, A of formula I-III, IA, IB, IIA, and IIB and R³ of formula ID is SO₃H. In one embodiment, A of formula I-III, IA, IB, IIA, and IIB and R³ of formula ID is SO₂NH₂. In one embodiment, A of formula I-III, IA, IB, IIA, and IIB and R³ of formula ID is SO₂NH(R⁴). In one embodiment, A of formula I-III, IA, IB, IIA, and IIB and R³ of formula ID is SO₂N(R⁴)₂. In one embodiment, A of formula I-III, IA, IB, IIA, and IIB and R³ of formula ID is NH₂. In one embodiment, A of formula I-III, IA, IB, IIA, and IIB and R³ of formula ID is NH(R⁴). In one embodiment, A of formula I-III, IA, IB, IIA, and IIB and R³ of formula ID is N(R⁴)₂. In one embodiment, A of formula I-III, IA, IB, IIA, and IIB and R³ of formula ID is CONH₂. In one embodiment, A of formula I-III, IA, IB, IIA, and IIB and R³ of formula ID is CONH(R⁴). In one embodiment, A of formula I-III, IA, IB, IIA, and IIB and R³ of formula ID is CO(N-heterocycle). In one embodiment, A of formula I-III, IA, IB, IIA, and IIB and R³ of formula ID is NO₂. In one embodiment, A of formula I-III, IA, IB, IIA, and IIB and R³ of formula ID is cyanate. In one embodiment, A of formula I-III, IA, IB, IIA, and IIB and R³ of formula ID is isocyanate. In one embodiment, A of formula I-III, IA, IB, IIA, and IIB and R³ of formula ID is thiocyanate. In one embodiment, A of formula I-III, IA, IB, IIA, and IIB and R³ of formula ID is isothiocyanate. In one embodiment, A of formula I-III, IA, IB, IIA, and IIB and R³ of formula ID is mesylate. In one embodiment, A of formula I-III, IA, IB, IIA, and IIB and R³ of formula ID is tosylate. In one embodiment, A of formula I-III, IA, IB, IIA, and IIB and R³ of formula ID is triflate. In one embodiment, A of formula I-III, IA, IB, IIA, and IIB and R³ of formula ID is PO(OH)₂. In one embodiment, A of formula I-III, IA, IB, IIA, and IIB and R³ of formula ID is OPO(OH)₂.

In one embodiment R⁴ is H, alkyl, haloalkyl, cycloalkyl, aryl or heteroaryl, wherein said alkyl, haloalkyl, cycloalkyl, aryl or heteroaryl groups are optionally substituted. Each represents a separate embodiment of this invention. In other embodiment, R⁴ is H. In other embodiments, R⁴ is alkyl. In other embodiments, the alkyl is methyl, ethyl, propyl, isopropyl, butyl, t-butyl, pentyl, neopentyl, iso-pentyl, hexyl, or heptyl, each represents a separate embodiment of this invention. In other embodiments, R⁴ is haloalkyl In another embodiment, the haloalkyl is CF₃, CF₂CF₃, iodomethyl, bromomethyl, bromoethyl, bromopropyl, each represents a separate embodiment of the invention. In other embodiments, R⁴ is cycloalkyl. In other embodiments the cycloalkyl is cyclobutyl, cyclopentyl, cyclohexyl. In various embodiments, the alkyl, haloalkyl, cycloalkyl, aryl or heteroaryl of R⁴ are further substituted by one or more groups selected from: halide, CN, CO₂H, OH, SH, NH₂, NO₂, CO₂—(C₁-C₆ alkyl) or O—(C₁-C₆ alkyl); each represents a separate embodiment of this invention.

In a particular embodiment of formulas I-VI, IA-IC, IIA, or IIB, Q¹ is hydrogen. In a particular embodiment of formulas I-VI, IA-IC, IIA, or IIB, Q¹ is CN. In a particular embodiment of formulas I-VI, IA-IC, IIA, or IIB, Q¹ is F. In a particular embodiment of formulas I-VI, IA-IC, IIA, or IIB, Q¹ is NCS. In a particular embodiment of formulas I-VI, IA-IC, IIA, or IIB, Q¹ is maleimide. In a particular embodiment of formulas I-VI, IA-IC, IIA, or IIB, Q¹ is NHCOOR. In a particular embodiment of formulas I-VI, IA-IC, IIA, or IIB, Q¹ is N(R)₂. In a particular embodiment of formulas I-VI, IA-IC, IIA, or IIB, Q¹ is CONHR. In a particular embodiment of formulas I-VI, IA-IC, IIA, or IIB, Q¹ is NHCOR. In a particular embodiment of formulas I-VI, IA-IC, IIA, or IIB, Q¹ is Cl. In a particular embodiment of formulas I-VI, IA-IC, IIA, or IIB, Q¹ is Br. In a particular embodiment of formulas I-VI, IA-IC, IIA, or IIB, Q¹ is I. In a particular embodiment of formulas I-VI, IA-IC, IIA, or IIB, Q¹ is NO₂. In a particular embodiment of formulas I-VI, IA-IC, IIA, or IIB, Q¹ is phenyl. In a particular embodiment of formulas I-VI, IA-IC, IIA, or IIB, Q¹ is 4-fluorophenyl. In a particular embodiment of formulas I-VI, IA-IC, IIA, or IIB, Q¹ is CF₃. In a particular embodiment of formulas I-VI, IA-IC, IIA, or IIB, Q¹ is substituted or unsubstituted alkyl. In a particular embodiment of formulas I-VI, IA-IC, IIA, or IIB, Q¹ is substituted or unsubstituted cycloalkyl. In a particular embodiment of formulas I-VI, IA-IC, IIA, or IIB, Q¹ is substituted or unsubstituted heterocycloalkyl. In a particular embodiment of formulas I-VI, IA-IC, IIA, or IIB, Q¹ is haloalkyl. In a particular embodiment of formulas I-VI, IA-IC, IIA, or IIB, Q¹ is substituted or unsubstituted aryl. In a particular embodiment of formulas I-VI, IA-IC, IIA, or IIB, Q¹ is hydroxyl. In a particular embodiment of formulas I-VI, IA-IC, IIA, or IIB, Q¹ is alkoxy. In a particular embodiment of formulas I-VI, IA-IC, IIA, or IIB, Q¹ is OR. In a particular embodiment of formulas I-VI, IA-IC, IIA, or IIB, Q¹ is arylalkyl. In a particular embodiment of formulas I-VI, IA-IC, IIA, or IIB, Q¹ is amine. In a particular embodiment of formulas I-VI, IA-IC, IIA, or IIB, Q¹ is amide. In a particular embodiment of formulas I-VI, IA-IC, IIA, and IIB, Q¹ is COOR. In a particular embodiment of formulas I-VI, IA-IC, IIA, or IIB, Q¹ is COR. In a particular embodiment of formulas I-VI, IA-IC, IIA, or IIB, Q¹ is keto.

In a particular embodiment of formulas I-VII, IA-IC, IIA, IIB, VIIA, or VIIB, Q² is CN. In a particular embodiment of formulas I-VII, IA-IC, IIA, IIB, VIIA, or VIIB, Q² is hydrogen. In a particular embodiment of formulas I-VII, IA-IC, IIA, IIB, VIIA, or VIIB, Q² is keto. In a particular embodiment of formulas I-VII, IA-IC, IIA, IIB, VIIA, or VIIB, Q² is NCS. In a particular embodiment of formulas I-VII, IA-IC, IIA, IIB, VIIA, or VIIB, Q² is maleimide. In a particular embodiment of formulas I-VII, IA-IC, IIA, IIB, VIIA, or VIIB, Q² is NHCOOR. In a particular embodiment of formulas I-VII, IA-IC, IIA, IIB, VIIA, or VIIB, Q² is N(R)₂. In a particular embodiment of formulas I-VII, IA-IC, IIA, IIB, VIIA, or VIIB, Q² is CONHR. In a particular embodiment of formulas I-VII, IA-IC, IIA, IIB, VIIA, or VIIB, Q² is NHCOR. In a particular embodiment of formulas I-VII, IA-IC, IIA, IIB, VIIA, or VIIB, Q² is F. In a particular embodiment of formulas I-VII, IA-IC, IIA, IIB, VIIA, or VIIB, Q² is Cl. In a particular embodiment of formulas I-VII, IA-IC, IIA, IIB, VIIA, or VIIB, Q² is Br. In a particular embodiment of formulas I-VII, IA-IC, IIA, IIB, VIIA, or VIIB, Q² is I. In a particular embodiment of formulas I-VII, IA-IC, IIA, IIB, VIIA, or VIIB, Q² is NO₂. In a particular embodiment of formulas I-VII, IA-IC, IIA, IIB, VIIA, or VIIB, Q² is phenyl. In a particular embodiment of formulas I-VII, IA-IC, IIA, IIB, VIIA, or VIIB, Q² is 4-fluorophenyl. In a particular embodiment of formulas I-VII, IA-IC, IIA, IIB, VIIA, or VIIB, Q² is CF₃. In a particular embodiment of formulas I-VII, IA-IC, IIA, IIB, VIIA, or VIIB, Q² is substituted or unsubstituted alkyl. In a particular embodiment of formulas I-VII, IA-IC, IIA, IIB, VIIA, or VIIB, Q² is substituted or unsubstituted cycloalkyl. In a particular embodiment of formulas I-VII, IA-IC, IIA, IIB, VIIA, or VIIB, Q² is substituted or unsubstituted heterocycloalkyl. In a particular embodiment of formulas I-VII, IA-IC, IIA, IIB, VIIA, or VIIB, Q² is haloalkyl. In a particular embodiment of formulas I-VII, IA-IC, IIA, IIB, VIIA, or VIIB, Q² is substituted or unsubstituted aryl. In a particular embodiment of formulas I-VII, IA-IC, IIA, IIB, VIIA, or VIIB, Q² is hydroxyl. In a particular embodiment of formulas I-VII, IA-IC, IIA, IIB, VIIA, or VIIB, Q² is alkoxy. In a particular embodiment of formulas I-VII, IA-IC, IIA, IIB, VIIA, or VIIB, Q² is OR. In a particular embodiment of formulas I-VII, IA-IC, IIA, IIB, VIIA, or VIIB, Q² is arylalkyl. In a particular embodiment of formulas I-VII, IA-IC, IIA, IIB, VIIA, or VIIB, Q² is amine. In a particular embodiment of formulas I-VII, IA-IC, IIA, IIB, VIIA, or VIIB, Q² is amide. In a particular embodiment of formulas I-VII, IA-IC, IIA, IIB, VIIA, or VIIB, Q² is COOR. In a particular embodiment of formulas I-VII, IA-IC, IIA, IIB, VIIA, or VIIB, Q² is COR.

In a particular embodiment of formulas I-VIII, IA-IC, IIA, IIB, VIIA, VIIB, VIIIA or VIIIB, Q³ is CN. In a particular embodiment of formulas I-VIII, IA-IC, IIA, IIB, VIIA, VIIB, VIIIA or VIIIB, Q³ is F. In a particular embodiment of formulas I-VIII, IA-IC, IIA, IIB, VIIA, VIIB, VIIIA or VIIIB, Q³ is NCS. In a particular embodiment of formulas I-VIII, IA-IC, IIA, IIB, VIIA, VIIB, VIIIA or VIIIB, Q³ is maleimide. In a particular embodiment of formulas I-VIII, IA-IC, IIA, IIB, VIIA, VIIB, VIIIA or VIIIB, Q³ is NHCOOR. In a particular embodiment of formulas I-VIII, IA-IC, IIA, IIB, VIIA, VIIB, VIIIA or VIIIB, Q³ is N(R)₂. In a particular embodiment of formulas I-VIII, IA-IC, IIA, IIB, VIIA, VIIB, VIIIA or VIIIB, Q³ is CONHR. In a particular embodiment of formulas I-VIII, IA-IC, IIA, IIB, VIIA, VIIB, VIIIA or VIIIB, Q³, is NHCOR. In a particular embodiment of formulas I-VIII, IA-IC, IIA, IIB, VIIA, VIIB, VIIIA or VIIIB, Q³ is hydrogen. In a particular embodiment of formulas I-VIII, IA-IC, IIA, IIB, VIIA, VIIB, VIIIA or VIIIB, Q³ is keto. In a particular embodiment of formulas I-VIII, IA-IC, IIA, IIB, VIIA, VIIB, VIIIA or VIIIB, Q³ is Cl. In a particular embodiment of formulas I-VIII, IA-IC, IIA, IIB, VIIA, VIIB, VIIIA or VIIIB, Q³ is Br. In a particular embodiment of formulas I-VIII, IA-IC, IIA, IIB, VIIA, VIIB, VIIIA or VIIIB, Q³ is I. In a particular embodiment of formulas I-VIII, IA-IC, IIA, IIB, VIIA, VIIB, VIIIA or VIIIB, Q³ is NO₂. In a particular embodiment of formulas I-VIII, IA-IC, IIA, IIB, VIIA, VIIB, VIIIA or VIIIB, Q³ is phenyl. In a particular embodiment of formulas I-VIII, IA-IC, IIA, IIB, VIIA, VIIB, VIIIA or VIIIB, Q³ is 4-fluorophenyl. In a particular embodiment of formulas I-VIII, IA-IC, IIA, IIB, VIIA, VIIB, VIIIA or VIIIB, Q³ is CF₃. In a particular embodiment of formulas I-VIII, IA-IC, IIA, IIB, VIIA, VIIB, VIIIA or VIIIB, Q³ is substituted or unsubstituted alkyl. In a particular embodiment of formulas I-VIII, IA-IC, IIA, IIB, VIIA, VIIB, VIIIA or VIIIB, Q³ is substituted or unsubstituted cycloalkyl. In a particular embodiment of formulas I-VIII, IA-IC, IIA, IIB, VIIA, VIIB, VIIIA or VIIIB, Q³ is substituted or unsubstituted heterocycloalkyl. In a particular embodiment of formulas I-VIII, IA-IC, IIA, IIB, VIIA, VIIB, VIIIA or VIIIB, Q³ is haloalkyl. In a particular embodiment of formulas I-VIII, IA-IC, IIA, IIB, VIIA, VIIB, VIIIA or VIIIB, Q³ is substituted or unsubstituted aryl. In a particular embodiment of formulas I-VIII, IA-IC, IIA, IIB, VIIA, VIIB, VIIIA or VIIIB, Q³ is hydroxyl. In a particular embodiment of formulas I-VIII, IA-IC, IIA, IIB, VIIA, VIIB, VIIIA or VIIIB, Q³ is alkoxy. In a particular embodiment of formulas I-VIII, IA-IC, IIA, IIB, VIIA, VIIB, VIIIA or VIIIB, Q³ is OR. In a particular embodiment of formulas I-VIII, IA-IC, IIA, IIB, VIIA, VIIB, VIIIA or VIIIB, Q³ is arylalkyl. In a particular embodiment of formulas I-VIII, IA-IC, IIA, IIB, VIIA, VIIB, VIIIA or VIIIB, Q³ is amine. In a particular embodiment of formulas I-VIII, IA-IC, IIA, IIB, VIIA, VIIB, VIIIA or VIIIB, Q³ is amide. In a particular embodiment of formulas I-VIII, IA-IC, IIA, IIB, VIIA, VIIB, VIIIA or VIIIB, Q³ is COOR. In a particular embodiment of formulas I-VIII, IA-IC, IIA, IIB, VIIA, VIIB, VIIIA or VIIIB, Q³ is COR.

In a particular embodiment of formulas I-IX, IA-IC, IIA, IIB, VIIA, VIIB, VIIIA, VIIIB, IXA or IXB, Q⁴ is CN. In a particular embodiment of formulas I-IX, IA-IC, IIA, IIB, VIIA, VIIB, VIIIA, VIIIB, IXA or IXB, Q⁴ is F. In a particular embodiment of formulas I-IX, IA-IC, IIA, IIB, VIIA, VIIB, VIIIA, VIIIB, IXA or IXB, Q⁴ is NCS. In a particular embodiment of formulas I-IX, IA-IC, IIA, IIB, VIIA, VIIB, VIIIA, VIIIB, IXA or IXB, Q⁴ is maleimide. In a particular embodiment of formulas I-IX, IA-IC, IIA, IIB, VIIA, VIIB, VIIIA, VIIIB, IXA or IXB, Q⁴ is NHCOOR. In a particular embodiment of formulas I-IX, IA-IC, IIA, IIB, VIIA, VIIB, VIIIA, VIIIB, IXA or IXB, Q⁴ is N(R)₂. In a particular embodiment of formulas I-IX, IA-IC, IIA, IIB, VIIA, VIIB, VIIIA, VIIIB, IXA or IXB, Q⁴ is CONHR. In a particular embodiment of formulas I-IX, IA-IC, IIA, IIB, VIIA, VIIB, VIIIA, VIIIB, IXA or IXB, Q⁴, is NHCOR. In a particular embodiment of formulas I-IX, IA-IC, IIA, IIB, VIIA, VIIB, VIIIA, VIIIB, IXA or IXB, Q⁴ is hydrogen. In a particular embodiment of formulas I-IX, IA-IC, IIA, IIB, VIIA, VIIB, VIIIA, VIIIB, IXA or IXB, Q⁴ is keto. In a particular embodiment of formulas I-IX, IA-IC, IIA, IIB, VIIA, VIIB, VIIIA, VIIIB, IXA or IXB B, Q⁴ is Cl. In a particular embodiment of formulas I-IX, IA-IC, IIA, IIB, VIIA, VIIB, VIIIA, VIIIB, IXA or IXB, Q⁴ is Br. In a particular embodiment of formulas I-IX, IA-IC, IIA, IIB, VIIA, VIIB, VIIIA, VIIIB, IXA or IXB, Q⁴ is I. In a particular embodiment of formulas I-IX, IA-IC, IIA, IIB, VIIA, VIIB, VIIIA, VIIIB, IXA or IXB, Q⁴ is NO₂. In a particular embodiment of formulas I-IX, IA-IC, IIA, IIB, VIIA, VIIB, VIIIA, VIIIB, IXA or IXB, Q⁴ is phenyl. In a particular embodiment of formulas I-IX, IA-IC, IIA, IIB, VIIA, VIIB, VIIIA, VIIIB, IXA or IXB, Q⁴ is 4-fluorophenyl. In a particular embodiment of formulas I-IX, IA-IC, IIA, IIB, VIIA, VIIB, VIIIA, VIIIB, IXA or IXB, Q⁴ is CF₃. In a particular embodiment of formulas I-IX, IA-IC, IIA, IIB, VIIA, VIIB, VIIIA, VIIIB, IXA or IXB, Q⁴ is substituted or unsubstituted alkyl. In a particular embodiment of formulas I-IX, IA-IC, IIA, IIB, VIIA, VIIB, VIIIA, VIIIB, IXA or IXB, Q⁴ is substituted or unsubstituted cycloalkyl. In a particular embodiment of formulas I-IX, IA-IC, IIA, IIB, VIIA, VIIB, VIIIA, VIIIB, IXA or IXB, Q⁴ is substituted or unsubstituted heterocycloalkyl. In a particular embodiment of formulas I-IX, IA-IC, IIA, IIB, VIIA, VIIB, VIIIA, VIIIB, IXA or IXB, Q⁴ is haloalkyl. In a particular embodiment of formulas I-IX, IA-IC, IIA, IIB, VIIA, VIIB, VIIIA, VIIIB, IXA or IXB, Q⁴ is substituted or unsubstituted aryl. In a particular embodiment of formulas I-IX, IA-IC, IIA, IIB, VIIA, VIIB, VIIIA, VIIIB, IXA or IXB, Q⁴ is hydroxyl. In a particular embodiment of formulas I-IX, IA-IC, IIA, IIB, VIIA, VIIB, VIIIA, VIIIB, IXA or IXB, Q⁴ is alkoxy. In a particular embodiment of formulas I-IX, IA-IC, IIA, IIB, VIIA, VIIB, VIIIA, VIIIB, IXA or IXB, Q⁴ is OR. In a particular embodiment of formulas I-IX, IA-IC, IIA, IIB, VIIA, VIIB, VIIIA, VIIIB, IXA or IXB, Q⁴ is arylalkyl. In a particular embodiment of formulas I-IX, IA-IC, IIA, IIB, VIIA, VIIB, VIIIA, VIIIB, IXA or IXB, Q⁴ is amine. In a particular embodiment of formulas I-IX, IA-IC, IIA, IIB, VIIA, VIIB, VIIIA, VIIIB, IXA or IXB, Q³ is amide. In a particular embodiment of formulas I-IX, IA-IC, IIA, IIB, VIIA, VIIB, VIIIA, VIIIB, IXA or IXB, Q⁴ is COOR. In a particular embodiment of formulas I-IX, IA-IC, IIA, IIB, VIIA, VIIB, VIIIA, VIIIB, IXA or IXB, Q⁴ is COR.

In a particular embodiment of formulas I, IA, IB, IC, ID, II, IIA, IIB, VII, VIIA, VIIB, VIII, VIIIA, VIIIB, IX, IXA or IXB, X is CH. In a particular embodiment of formulas I, IA, IB, IC, ID, II, IIA, IIB, VII, VIIA, VIIB, VIII, VIIIA, VIIIB, IX, IXA or IXB, X is N.

In a particular embodiment of formulas I-IX, IA, IB, IC, ID, IIA, IIB, VIIA, VIIB, VIIIA, VIIIB, IXA, or IXB, Y is H. In a particular embodiment of formulas I-IX, IA, IB, IC, ID, IIA, IIB, VIIA, VIIB, VIIIA, VIIIB, IXA, or IXB, Y is CF₃. In a particular embodiment of formulas I-IX, IA, IB, IC, ID, IIA, IIB, VIIA, VIIB, VIIIA, VIIIB, IXA, or IXB, Y is F. In a particular embodiment of formulas I-IX, IA, IB, IC, ID, IIA, IIB, VIIA, VIIB, VIIIA, VIIIB, IXA, or IXB, Y is I. In a particular embodiment of formulas I-IX, IA, IB, IC, ID, IIA, IIB, VIIA, VIIB, VIIIA, VIIIB, IXA, or IXB, Y is Br. In a particular embodiment of formulas I-IX, IA, IB, IC, ID, IIA, IIB, VIIA, VIIB, VIIIA, VIIIB, IXA, or IXB, Y is Cl. In a particular embodiment of formulas I-IX, IA, IB, IC, ID, IIA, IIB, VIIA, VIIB, VIIIA, VIIIB, IXA, or IXB, Y is CN. In a particular embodiment of formulas I-IX, IA, IB, IC, ID, IIA, IIB, VIIA, VIIB, VIIIA, VIIIB, IXA, or IXB, Y is C(R)₃.

In a particular embodiment of formulas I-IX, IA, IB, IC, ID, IIA, IIB, VIIA, VIIB, VIIIA, VIIIB, IXA, or IXB, Z is H. In a particular embodiment of formulas I-IX, IA, IB, IC, ID, IIA, IIB, VIIA, VIIB, VIIIA, VIIIB, IXA, or IXB, Z is NO₂. In a particular embodiment of formulas I-IX, IA, IB, IC, ID, IIA, IIB, VIIA, VIIB, VIIIA, VIIIB, IXA, or IXB, Z is CN. In a particular embodiment of formulas I-IX, IA, IB, IC, ID, IIA, IIB, VIIA, VIIB, VIIIA, VIIIB, IXA, or IXB, Z is a halide. In a particular embodiment of formulas I-VII, IA, IB, IC, ID, IIA, IIB, VIIA, or VIIB, Z is F. In a particular embodiment of formulas I-IX, IA, IB, IC, ID, IIA, IIB, VIIA, VIIB, VIIIA, VIIIB, IXA, or IXB, Z is Cl. In a particular embodiment of formulas I-IX, IA, IB, IC, ID, IIA, IIB, VIIA, VIIB, VIIIA, VIIIB, IXA, or IXB, Z is Br. In a particular embodiment of formulas I-IX, IA, IB, IC, ID, IIA, IIB, VIIA, VIIB, VIIIA, VIIIB, IXA, or IXB, Z is I. In a particular embodiment of formulas I-IX, IA, IB, IC, ID, IIA, IIB, VIIA, VIIB, VIIIA, VIIIB, IXA, or IXB, Z is COOH. In a particular embodiment of formulas I-IX, IA, IB, IC, ID, IIA, IIB, VIIA, VIIB, VIIIA, VIIIB, IXA, or IXB, Z is COR. In a particular embodiment of formulas I-IX, IA, IB, IC, ID, IIA, IIB, VIIA, VIIB, VIIIA, VIIIB, IXA, or IXB, Z is NHCOR. In a particular embodiment of formulas I-IX, IA, IB, IC, ID, IIA, IIB, VIIA, VIIB, VIIIA, VIIIB, IXA, or IXB, Z is CONHR.

In a particular embodiment of formulas I I-IX, IA, IB, IC, ID, IIA, IIB, VIIA, VIIB, VIIIA, VIIIB, IXA, or IXB, Y and Z forms a fused ring with the phenyl. In other embodiments, the fused ring with the phenyl is a 5 to 8 membered ring. In other embodiments, the fused ring with the phenyl is a 5 or 6 membered ring. In other embodiments, the ring is a carbocyclic or heterocyclic. In other embodiments, Y and Z form together with the phenyl to form a naphthyl, quinolinyl, benzimidazolyl, indazolyl, indolyl, isoindolyl, indenyl, or quinazolinyl. In a particular embodiment, Y and Z form together with the phenyl to form a quinazolin-6-yl ring system.

In a particular embodiment of formulas I, II, IV, V, VI, VII, VIII, IX IA, IB, IC, ID, IIA, IIB, VIIA, VIIB, VIIIA, VIIIB, IXA or IXB R¹ is H. In a particular embodiment of formulas I, II, IV, V, VI, VII, VIII, IX IA, IB, IC, ID, IIA, IIB, VIIA, VIIB, VIIIA, VIIIB, IXA or IXB, R¹ is CH₃. In a particular embodiment of formulas I, II, IV, V, VI, VII, VIII, IX IA, IB, IC, ID, IIA, IIB, VIIA, VIIB, VIIIA, VIIIB, IXA or IXB, R¹ is CH₂F. In a particular embodiment of formulas I, II, IV, V, VI, VII, VIII, IX IA, IB, IC, ID, IIA, IIB, VIIA, VIIB, VIIIA, VIIIB, IXA or IXB, R¹ is CHF₂. In a particular embodiment of formulas I, II, IV, V, VI, VII, VIII, IX IA, IB, IC, ID, IIA, IIB, VIIA, VIIB, VIIIA, VIIIB, IXA or IXB, R₁ is CF₃. In a particular embodiment of formulas I, II, IV, V, VI, VII, VIII, IX IA, IB, IC, ID, IIA, IIB, VIIA, VIIB, VIIIA, VIIIB, IXA or IXB, R¹ is CH₂CH₃. In a particular embodiment of formulas I, II, IV, V, VI, VII, VIII, IX IA, IB, IC, ID, IIA, IIB, VIIA, VIIB, VIIIA, VIIIB, IXA or IXB, R¹ is CF₂CF₃.

In a particular embodiment of formulas I, II, IV, V, VI, VII, VIII, IX, IA, IB, IC, ID, IIA, IIB, VIIA, VIIB, VIIIA, VIIIB, IXA or IXB, T is H. In a particular embodiment of formulas I, II, IV, V, VI, VII, VIII, IX, IA, IB, IC, ID, IIA, IIB, VIIA, VIIB, VIIIA, VIIIB, IXA or IXB, T is OH. In a particular embodiment of formulas I, II, IV, V, VI, VII, VIII, IX, IA, IB, IC, ID, IIA, IIB, VIIA, VIIB, VIIIA, VIIIB, IXA or IXB, T is OR. In a particular embodiment of formulas I, II, IV, V, VI, VII, VIII, IX, IA, IB, IC, ID, IIA, IIB, VIIA, VIIB, VIIIA, VIIIB, IXA or IXB, T is OCOR In a particular embodiment of formulas I, II, IV, V, VI, VII, VIII, IX, IA, IB, IC, ID, IIA, IIB, VIIA, VIIB, VIIIA, VIIIB, IXA or IXB, T is CH₃. In a particular embodiment of formulas I, II, IV, V, VI, VII, VIII, IX, IA, IB, IC, ID, IIA, IIB, VIIA, VIIB, VIIIA, VIIIB, IXA or IXB, T is —NHCOCH₃. In a particular embodiment of formulas I, II, IV, V, VI, VII, VIII, IX, IA, IB, IC, ID, IIA, IIB, VIIA, VIIB, VIIIA, VIIIB, IXA or IXB, T is NHCOR.

In a particular embodiment of formulas I, II, IV, V, VI, VII, VIII, IX, IA, IB, IC, ID, IIA, IIB, VIIA, VIIB, VIIIA, VIIIB, IXA or IXB, T and R¹ form a 3-8 carbocyclic or heterocyclic ring. In other embodiments, T and R¹ form a 3, 4, 5, 6, 7, or 8 membered carbocyclic or heterocyclic ring. Each represents a separate embodiment of this invention. In some embodiments T and R¹ form a carbocyclic ring such as cyclopropyl, cyclobutyl, cyclopentyl, cyclohexyl, etc. In some embodiments T and R¹ form a heterocyclic ring such as piperidine, pyridine, furan, thiphene, pyrrole, pyrazole, pyrimidine, etc.

In a particular embodiment of formulas I-IX, IA, IB, IC, ID, IIA, IIB, VIIA, VIIB, VIIIA, VIIIB, IXA or IXB, R is H. In a particular embodiment of formulas I-IX, IA, IB, IC, ID, IIA, IIB, VIIA, VIIB, VIIIA, VIIIB, IXA, R is alkyl. In a particular embodiment of formulas I-IX, IA, IB, IC, ID, IIA, IIB, VIIA, VIIB, VIIIA, VIIIB, IXA, R is alkenyl. In a particular embodiment of formulas I-IX, IA, IB, IC, ID, IIA, IIB, VIIA, VIIB, VIIIA, VIIIB, IXA, R is haloalkyl. In a particular embodiment of formulas I-IX, IA, IB, IC, ID, IIA, IIB, VIIA, VIIB, VIIIA, VIIIB, IXA, R is alcohol. In a particular embodiment of formulas I-VII, IA, IB, IC, ID, IIA, IIB, VIIA, or VIIB, R is CH₂CH₂OH. In a particular embodiment of formulas I-IX, IA, IB, IC, ID, IIA, IIB, VIIA, VIIB, VIIIA, VIIIB, IXA, R is CF₃. In a particular embodiment of formulas I-VII, IA, IB, IC, ID, IIA, IIB, VIIA, or VIIB, R is CH₂Cl. In a particular embodiment of formulas I-IX, IA, IB, IC, ID, IIA, IIB, VIIA, VIIB, VIIIA, VIIIB, IXA, R is CH₂CH₂Cl. In a particular embodiment of formulas I-IX, IA, IB, IC, ID, IIA, IIB, VIIA, VIIB, VIIIA, VIIIB, IXA, R is aryl. In a particular embodiment of formulas I-IX, IA, IB, IC, ID, IIA, IIB, VIIA, VIIB, VIIIA, VIIIB, IXA, R is F. In a particular embodiment of formulas I-IX, IA, IB, IC, ID, IIA, IIB, VIIA, VIIB, VIIIA, VIIIB, IXA, R is Cl. In a particular embodiment of formulas I-IX, IA, IB, IC, ID, IIA, IIB, VIIA, VIIB, VIIIA, VIIIB, IXA, R is Br. In a particular embodiment of formulas I-IX, IA, IB, IC, ID, IIA, IIB, VIIA, VIIB, VIIIA, VIIIB, IXA, R is I. In a particular embodiment of formulas I-IX, IA, IB, IC, ID, IIA, IIB, VIIA, VIIB, VIIIA, VIIIB, IXA, R is OH.

In a particular embodiment of formula IV, Q¹ is H, CN, CF₃, phenyl, 4-fluorophenyl, F, Br, Cl, I, COMe, NHCOOMe, NHCOMe or NHCOOC(CH₃)₃.

In a particular embodiment of formula V, Q¹ is H, CN, CF₃, phenyl, 4-fluorophenyl, F, Br, Cl, I, COMe, NHCOOMe, NHCOMe or NHCOOC(CH₃)₃.

In a particular embodiment of formula VI, Q¹ is H, CN, CF₃, phenyl, 4-fluorophenyl, F, Br, Cl, I, COMe, NHCOOMe, NHCOMe or NHCOOC(CH₃)₃.

In a particular embodiment of formula IV, Q² is H, CN, CF₃, phenyl, 4-fluorophenyl, F, Br, Cl, I, COMe, NHCOOMe, NHCOMe or NHCOOC(CH₃)₃.

In a particular embodiment of formula V, Q² is H, CN, CF₃, phenyl, 4-fluorophenyl, F, Br, Cl, I, COMe, NHCOOMe, NHCOMe or NHCOOC(CH₃)₃.

In a particular embodiment of formula VI, Q² is H, CN, CF₃, phenyl, 4-fluorophenyl, F, Br, Cl, I, COMe, NHCOOMe, NHCOMe or NHCOOC(CH₃)₃.

In a particular embodiment of formula VII, Q² is H, CN, CF₃, phenyl, 4-fluorophenyl, F, Br, Cl, I, COMe, NHCOOMe, NHCOMe or NHCOOC(CH₃)₃.

In a particular embodiment of formula VIIA, Q² is H, CN, CF₃, phenyl, 4-fluorophenyl, F, Br, Cl, I, COMe, NHCOOMe, NHCOMe or NHCOOC(CH₃)₃.

In a particular embodiment of formula VIIB, Q² is H, CN, CF₃, phenyl, 4-fluorophenyl, F, Br, Cl, I, COMe, NHCOOMe, NHCOMe or NHCOOC(CH₃)₃.

In a particular embodiment of formula IV, Q³ is H, CN, CF₃, phenyl, 4-fluorophenyl, F, Br, Cl, I, COMe, NHCOOMe, NHCOMe or NHCOOC(CH₃)₃.

In a particular embodiment of formula V, Q³ is H, CN, CF₃, phenyl, 4-fluorophenyl, F, Br, Cl, I, COMe, NHCOOMe, NHCOMe or NHCOOC(CH₃)₃.

In a particular embodiment of formula VI, Q³ is H, CN, CF₃, phenyl, 4-fluorophenyl, F, Br, Cl, I, COMe, NHCOOMe, NHCOMe or NHCOOC(CH₃)₃.

In a particular embodiment of formula VII, Q³ is H, CN, CF₃, phenyl, 4-fluorophenyl, F, Br, Cl, I, COMe, NHCOOMe, NHCOMe or NHCOOC(CH₃)₃.

In a particular embodiment of formula VIII, Q³ is H, CN, CF₃, phenyl, 4-fluorophenyl, F, Br, Cl, I, COMe, NHCOOMe, NHCOMe or NHCOOC(CH₃)₃.

In a particular embodiment of formula IV, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula V, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula VI, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula VII, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula VIIA, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula VIIB, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula VIII, VIIIA, or VIIIB, Q⁴ is H, CN, CF₃, phenyl, 4-fluorophenyl, F, Br, Cl, I, COMe, NHCOOMe, NHCOMe or NHCOOC(CH₃)₃.

In a particular embodiment of formula IX, IXA, or IXB, Q⁴ is H, CN, CF₃, phenyl, 4-fluorophenyl, F, Br, Cl, I, COMe, NHCOOMe, NHCOMe or NHCOOC(CH₃)₃.

The invention encompasses a selective androgen receptor degrader (SARD) of compound 1068.

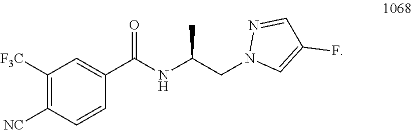 [SMILES: C[C@@H](Cn1cc(F)cn1)NC(=O)c1ccc(C#N)c(C(F)(F)F)c1]

As used herein, the term “heterocycle” or “heterocyclic ring” group refers to a ring structure comprising in addition to carbon atoms, at least one atom of sulfur, oxygen, nitrogen or any combination thereof, as part of the ring. The heterocycle may be a 3-12 membered ring; 4-8 membered ring; a 5-7 membered ring; or a 6 membered ring. Preferably, the heterocycle is a 5 to 6 membered ring. Typical examples of heterocycles include, but are not limited to, piperidine, pyridine, furan, thiophene, pyrrole, pyrrolidine, pyrazole, pyrazine, piperazine or pyrimidine. Examples of C₅-C₈ heterocyclic rings include pyran, dihydropyran, tetrahydropyran, dihydropyrrole, tetrahydropyrrole, pyrazine, dihydropyrazine, tetrahydropyrazine, pyrimidine, dihydropyrimidine, tetrahydropyrimidone, pyrazole, dihydropyrazole, tetrahydropyrazole, triazole, tetrazole, piperidine, piperazine, pyridine, dihydropyridine, tetrahydropyridine, morpholine, thiomorpholine, furan, dihydrofuran, tetrahydrofuran, thiophene, dihydrothiophene, tetrahydrothiophene, thiazole, imidazole, isoxazole, and the like. In one embodiment, the heterocyclic ring includes, but is not limited to, indole, indoline, benzotriazole, indazole, pyrrolo-pyridine, benzimidazoles, isoquinolines and quinolines, pyridine, pyrimidine, pyrrole, pyrroline, pyrrolidine, pyrazole, pyrazoline, pyrazolidine, imidazole, imidazoline, imidazolidine, triazole, tetrazole, morpholine. The heterocycle ring may be fused to another saturated or unsaturated cycloalkyl or a saturated or unsaturated heterocyclic ring. When the heterocycle ring is substituted, the substituents include at least one of halogen, haloalkyl, hydroxyl, alkoxy, carbonyl, amido, alkylamido, dialkylamido, cyano, nitro, CO₂H, amino, alkylamino, dialkylamino, carboxyl, thiol, or thioalkyl.

The term “cycloalkyl” refers to anon-aromatic, monocyclic or polycyclic ring comprising carbon and hydrogen atoms. A cycloalkyl group can have one or more carbon-carbon double bonds in the ring so long as the ring is not rendered aromatic by their presence. Examples of cycloalkyl groups include, but are not limited to, (C₃-C₇) cycloalkyl groups, such as cyclopropyl, cyclobutyl, cyclopentyl, cyclohexyl, and cycloheptyl, and saturated cyclic and bicyclic terpenes and (C₃-C₇) cycloalkenyl groups, such as cyclopropenyl, cyclobutenyl, cyclopentenyl, cyclohexenyl, and cycloheptenyl, and unsaturated cyclic and bicyclic terpenes. Examples of C₅-C₈ carbocyclic include cyclopentane, cyclopentene, cyclohexane, and cyclohexene rings. A cycloalkyl group can be unsubstituted or substituted by at least one substituent. Preferably, the cycloalkyl group is a monocyclic ring or bicyclic ring.

The term “alkyl” refers to a saturated aliphatic hydrocarbon, including straight-chained and branched-chained. Typically, the alkyl group has 1-12 carbons, 1-7 carbons, 1-6 carbons, or 1-4 carbon atoms. A branched alkyl is an alkyl substituted by alkyl side chains of 1 to 5 carbons. The branched alkyl may have an alkyl substituted by a C₁-C₅ haloalkyl. Additionally, the alkyl group may be substituted by at least one of halogen, haloalkyl, hydroxyl, alkoxy carbonyl, amido, alkylamido, dialkylamido, nitro, CN, amino, alkylamino, dialkylamino, carboxyl, thio or thioalkyl.

An “arylalkyl” group refers to an alkyl bound to an aryl, wherein alkyl and aryl are as defined herein. An example of an arylalkyl group is a benzyl group.

An “alkenyl” group refers to an unsaturated hydrocarbon, including straight chain and branched chain having one or more double bonds. The alkenyl group may have 2-12 carbons, preferably the alkenyl group has 2-6 carbons or 2-4 carbons. Examples of alkenyl groups include, but are not limited to, ethenyl, propenyl, butenyl, cyclohexenyl, etc. The alkenyl group may be substituted by at least one halogen, hydroxy, alkoxy carbonyl, amido, alkylamido, dialkylamido, nitro, amino, alkylamino, dialkylamino, carboxyl, thio, or thioalkyl.

As used herein ther term “aryl” group refers to an aromatic group having at least one carbocyclic aromatic group or heterocyclic aromatic group, which may be unsubstituted or substituted. When present, substituents include, but are not limited to, at least one halogen, haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like. The aryl group may be a 4-12 membered ring, preferably the aryl group is a 4-8 membered ring. Also the aryl group may be a 6 or 5 membered ring.

The term “heteroaryl” refers to an aromatic group having at least one heterocyclic aromatic ring. In one embodiment, the heteroaryl comprises at least one heteroatom such as sulfur, oxygen, nitrogen, silicon, phosphorous or any combination thereof, as part of the ring. In another embodiment, the heteroaryl may be unsubstituted or substituted by one or more groups selected from halogen, aryl, heteroaryl, cyano, haloalkyl, hydroxy, alkoxy carbonyl, amido, alkylamido, dialkylamido, nitro, amino, alkylamino, dialkylamino, carboxy or thio or thioalkyl. Nonlimiting examples of heteroaryl rings are pyranyl, pyrrolyl, pyrazinyl, pyrimidinyl, pyrazolyl, pyridinyl, furanyl, thiophenyl, thiazolyl, indolyl, imidazolyl, isoxazolyl, and the like. In one embodiment, the heteroaryl group is a 5-12 membered ring. In one embodiment, the heteroaryl group is a five membered ring. In one embodiment, the heteroaryl group is a six membered ring. In another embodiment, the heteroaryl group is a 5-8 membered ring. In another embodiment, the heteroaryl group comprises of 1-4 fused rings. In one embodiment, the heteroaryl group is 1,2,3-triazole. In one embodiment the heteroaryl is a pyridyl. In one embodiment the heteroaryl is a bipyridyl. In one embodiment the heteroaryl is a terpyridyl.

As used herein, the term “haloalkyl” group refers to an alkyl group that is substituted by one or more halogen atoms, e.g. by F, Cl, Br or I.

A “hydroxyl” group refers to an OH group. It is understood by a person skilled in the art that when T, Q¹, Q², Q³, or Q⁴, in the compounds of the present invention is OR, then R is not OH.

The term “halogen” or “halo” or “halide” refers to a halogen; F, Cl, Br or I.

In one embodiment, this invention provides the compounds and/or its use and/or its derivative, optical isomer, isomer, metabolite, pharmaceutically acceptable salt, pharmaceutical product, hydrate, N-oxide, prodrug, polymorph, crystal or combinations thereof.

In one embodiment, the methods of this invention make use of “pharmaceutically acceptable salts” of the compounds, which may be produced, by reaction of a compound of this invention with an acid or base.

The compounds of the invention may be converted into pharmaceutically acceptable salts. A pharmaceutically acceptable salt may be produced by reaction of a compound with an acid or base.

Suitable pharmaceutically acceptable salts of amines may be prepared from an inorganic acid or from an organic acid. Examples of inorganic salts of amines include, but are not limited to, bisulfates, borates, bromides, chlorides, hemisulfates, hydrobromates, hydrochlorates, 2-hydroxyethylsulfonates (hydroxyethanesulfonates), iodates, iodides, isothionates, nitrates, persulfates, phosphates, sulfates, sulfamates, sulfanilates, sulfonic acids (alkylsulfonates, arylsulfonates, halogen substituted alkylsulfonates, halogen substituted arylsulfonates), sulfonates, or thiocyanates.

Examples of organic salts of amines may be selected from aliphatic, cycloaliphatic, aromatic, araliphatic, heterocyclic, carboxylic and sulfonic classes of organic acids, examples of which are acetates, arginines, aspartates, ascorbates, adipates, anthranilates, algenates, alkane carboxylates, substituted alkane carboxylates, alginates, benzenesulfonates, benzoates, bisulfates, butyrates, bicarbonates, bitartrates, carboxylates, citrates, camphorates, camphorsulfonates, cyclohexylsulfamates, cyclopentanepropionates, calcium edetates, camsylates, carbonates, clavulanates, cinnamates, dicarboxylates, digluconates, dodecylsulfonates, dihydrochlorides, decanoates, enanthuates, ethanesulfonates, edetates, edisylates, estolates, esylates, fumarates, formates, fluorides, galacturonates, gluconates, glutamates, glycolates, glucorates, glucoheptanoates, glycerophosphates, gluceptates, glycollylarsanilates, glutarates, glutamates, heptanoates, hexanoates, hydroxymaleates, hydroxycarboxlic acids, hexylresorcinates, hydroxybenzoates, hydroxynaphthoates, hydrofluorates, lactates, lactobionates, laurates, malates, maleates, methylenebis(beta-oxynaphthoate), malonates, mandelates, mesylates, methane sulfonates, methylbromides, methylnitrates, methylsulfonates, monopotassium maleates, mucates, monocarboxylates, nitrates, naphthalenesulfonates, 2-naphthalenesulfonates, nicotinates, napsylates, N-methylglucamines, oxalates, octanoates, oleates, pamoates, phenylacetates, picrates, phenylbenzoates, pivalates, propionates, phthalates, pectinates, phenylpropionates, palmitates, pantothenates, polygalacturates, pyruvates, quinates, salicylates, succinates, stearates, sulfanilates, subacetates, tartarates, theophyllineacetates, p-toluenesulfonates (tosylates), trifluoroacetates, terephthalates, tannates, teoclates, trihaloacetates, triethiodide, tricarboxylates, undecanoates and valerates. Examples of inorganic salts of carboxylic acids or phenols may be selected from ammonium, alkali metals, and alkaline earth metals. Alkali metals include, but are not limited to, lithium, sodium, potassium, or cesium. Alkaline earth metals include, but are not limited to, calcium, magnesium, aluminium; zinc, barium, cholines, or quaternary ammoniums. Examples of organic salts of carboxylic acids or phenols may be selected from arginine, organic amines to include aliphatic organic amines, alicyclic organic amines, aromatic organic amines, benzathines, t-butylamines, benethamines (N-benzylphenethylamine), dicyclohexylamines, dimethylamines, diethanolamines, ethanolamines, ethylenediamines, hydrabamines, imidazoles, lysines, methylamines, meglumines, N-methyl-D-glucamines, N,N′-dibenzylethylenediamines, nicotinamides, organic amines, ornithines, pyridines, picolines, piperazines, procaine, tris(hydroxymethyl)methylamines, triethylamines, triethanolamines, trimethylamines, tromethamines and ureas.

In various embodiments, the pharmaceutically acceptable salts of the compounds of this invention include: HCl salt, oxalic acid salt, L-(+)-tartaric acid salt, HBr salt and succinic acid salt. Each represents a separate embodiment of this invention. E.g., the tartaric acid salt of 1002 (1002 Tart.) is exemplified in Table 1.

Salts may be formed by conventional means, such as by reacting the free base or free acid form of the product with one or more equivalents of the appropriate acid or base in a solvent or medium in which the salt is insoluble or in a solvent such as water, which is removed in vacuo or by freeze drying or by exchanging the ions of a existing salt for another ion or suitable ion-exchange resin.

The methods of the invention may use an uncharged compound or a pharmaceutically acceptable salt of the compound. In particular, the methods use pharmaceutically acceptable salts of compounds of formulas I-IX, IA, IB, IC, ID, IIA, IIB, VIIA, VIIB, VIIIA, VIIIB, IXA or IXB. The pharmaceutically acceptable salt may be an amine salt or a salt of a phenol of the compounds of formulas I-IX, IA, IB, IC, ID, IIA, IIB, VIIA, VIIB, VIIIA, VIIIB, IXA or IXB.

The methods of the invention may use an uncharged compound or a pharmaceutically acceptable salt of the compound. In particular, the methods use pharmaceutically acceptable salts of compounds of formulas 44-46, 98, 300-308, 1050-1064, and 1068. The pharmaceutically acceptable salt may be an amine salt or a salt of a phenol of the compounds of formulas 44-46, 98, 300-308, 1050-1064, and 1068.

In one embodiment, the methods of this invention make use of a free base, free acid, non charged or non-complexed compounds of formulas I-IX, IA, IB, IC, ID, IIA, IIB, VIIA, VIIB, VIIIA, VIIIB, IXA or IXB, and/or its isomer, pharmaceutical product, hydrate, polymorph, or combinations thereof.

In one embodiment, the methods of this invention make use of a free base, free acid, non charged or non-complexed compounds of formulas 44-46, 98, 300-308, 1050-1064, and 1068, and/or its isomer, pharmaceutical product, hydrate, polymorph, or combinations thereof.

In one embodiment, the methods of this invention make use of an optical isomer of a compound of formulas I-IX, IA, IB, IC, ID, IIA, IIB, VIIA, VIIB, VIIIA, VIIIB, IXA or IXB. In one embodiment, the methods of this invention make use of an isomer of a compound of formulas I-IX, IA, IB, IC, ID, IIA, IIB, VIIA, VIIB, VIIIA, VIIIB, IXA or IXB. In one embodiment, the methods of this invention make use of a pharmaceutical product of a compound of formulas I-IX, IA, IB, IC, ID, IIA, IIB, VIIA, VIIB, VIIIA, VIIIB, IXA or IXB. In one embodiment, the methods of this invention make use of a hydrate of a compound of I-IX, IA, IB, IC, ID, IIA, IIB, VIIA, VIIB, VIIIA, VIIIB, IXA or IXB. In one embodiment, the methods of this invention make use of a polymorph of a compound of formulas I-IX, IA, IB, IC, ID, IIA, IIB, VIIA, VIIB, VIIIA, VIIIB, IXA or IXB. In one embodiment, the methods of this invention make use of a metabolite of a compound of formulas I-IX, IA, IB, IC, ID, IIA, IIB, VIIA, VIIB, VIIIA, VIIIB, IXA or IXB. In another embodiment, the methods of this invention make use of a composition comprising a compound of formulas I-IX, IA, IB, IC, ID, IIA, IIB, VIIA, VIIB, VIIIA, VIIIB, IXA or IXB, as described herein, or, in another embodiment, a combination of isomer, metabolite, pharmaceutical product, hydrate, polymorph of a compound of formulas I-IX, IA, IB, IC, ID, IIA, IIB, VIIA, VIIB, VIIIA, VIIIB, IXA or IXB.

In one embodiment, the methods of this invention make use of an optical isomer of a compound of formulas 44-46, 98, 300-308, 1050-1064, and 1068. In one embodiment, the methods of this invention make use of an isomer of a compound of formulas 44-46, 98, 300-308, 1050-1064, and 1068. In one embodiment, the methods of this invention make use of a pharmaceutical product of a compound of formulas 44-46, 98, 300-308, 1050-1064, and 1068. In one embodiment, the methods of this invention make use of a hydrate of a compound of formulas 44-46, 98, 300-308, 1050-1064, and 1068. In one embodiment, the methods of this invention make use of a polymorph of a compound of formulas 44-46, 98, 300-308, 1050-1064, and 1068. In one embodiment, the methods of this invention make use of a metabolite of a compound of formulas 44-46, 98, 300-308, 1050-1064, and 1068. In another embodiment, the methods of this invention make use of a composition comprising a compound of formulas 44-46, 98, 300-308, 1050-1064, and 1068, as described herein, or, in another embodiment, a combination of isomer, metabolite, pharmaceutical product, hydrate, polymorph of a compound of formulas 44-46, 98, 300-308, 1050-1064, and 1068.

As used herein, the term “isomer” includes, but is not limited to, optical isomers, structural isomers, or conformational isomers.

The term “isomer” is meant to encompass optical isomers of the SARD compound. It will be appreciated by those skilled in the art that the SARDs of the present invention contain at least one chiral center. Accordingly, the compounds may exist as optically-active (such as an (R) isomer or (S) isomer) or racemic forms. Optically active compounds may exist as enantiomerically enriched mixtures. Some compounds may also exhibit polymorphism. It is to be understood that the present invention encompasses any racemic, optically active, polymorphic, or stereroisomeric form, or mixtures thereof. Thus, the invention may encompass SARD compounds as pure (R)-isomers or as pure (S)-isomers. It is known in the art how to prepare optically active forms. For example, by resolution of the racemic form by recrystallization techniques, by synthesis from optically active starting materials, by chiral synthesis, or by chromatographic separation using a chiral stationary phase.

Compounds of the invention may be hydrates of the compounds. As used herein, the term “hydrate” includes, but is not limited to, hemihydrate, monohydrate, dihydrate, or trihydrate. The invention also includes use of N-oxides of the amino substituents of the compounds described herein.

This invention provides, in other embodiments, use of metabolites of the compounds as herein described. In one embodiment, “metabolite” means any substance produced from another substance by metabolism or a metabolic process.

In one embodiment, the compounds of this invention are prepared according to Examples 1, 3-6, and 12.

### Biological Activity of Selective Androgen Receptor Degraders

The invention provides a method of treating prostate cancer (PCa) or increasing the survival of a male subject suffering from prostate cancer comprising administering to the subject a therapeutically effective amount of a compound or its pharmaceutically acceptable salt, represented by a compound of formula I:

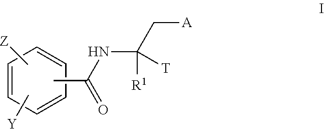

wherein

T is H, OH, OR, OCOR, CH₃, —NHCOCH₃, or NHCOR;

R¹ is H, CH₃, CH₂F, CHF₂, CF₃, CH₂CH₃, or CF₂CF₃;

or T and R¹ form a 3-8 carbocyclic or heterocyclic ring;

Y is H, CF₃, F, I, Br, Cl, CN, or C(R)₃;

Z H, is NO₂, CN, halide, COOH, COR, NHCOR, CONHR, or Y and Z form a 5 to 8 membered ring;

X is CH or N;

R is H, alkyl, alkenyl, haloalkyl, alcohol, CH₂CH₂OH, CF₃, CH₂Cl, CH₂CH₂Cl, aryl, F, Cl, Br, I, or OH;

A is R² or R³;

R² is an N-heterocyclic ring, optionally substituted with at least one of Q¹, Q², Q³, and Q⁴, each independently selected from hydrogen, keto, substituted or unsubstituted linear or branched alkyl, substituted or unsubstituted cycloalkyl, substituted or unsubstituted heterocycloalkyl, haloalkyl, CF₃, substituted or unsubstituted aryl, substituted or unsubstituted phenyl, F, Cl, Br, I, CN, NO₂, hydroxyl, alkoxy, OR, arylalkyl, NCS, maleimide, NHCOOR, N(R)₂, NHCOR, CONHR, COOR or COR;

R³ is NHR², halide, N₃, OR⁴, CF₃, COR⁴, COCl, COOCOR⁴, COOR⁴, OCOR⁴, OCONHR⁴, NHCOOR⁴, NHCONHR⁴, OCOOR⁴, CN, CONH₂, CONH(R⁴), CON(R⁴)₂, SR⁴, SO₂R⁴, SOR⁴ SO₃H, SO₂NH₂, SO₂NH(R⁴), SO₂N(R⁴)₂, NH₂, NH(R⁴), N(R⁴)₂, CO(N-heterocycle), C(O)(C₁-C₁₀)alkyl, NO₂, cyanate, isocyanate, thiocyanate, isothiocyanate, mesylate, tosylate, triflate, PO(OH)₂ or OPO(OH)₂; and

R⁴ is H, alkyl, haloalkyl, cycloalkyl, aryl or heteroaryl, wherein said alkyl, haloalkyl, cycloalkyl, aryl or heteroaryl groups are optionally substituted;

or its optical isomer, isomer, pharmaceutically acceptable salt, pharmaceutical product, polymorph, hydrate or any combination thereof.

The invention provides a method of treating prostate cancer (PCa) or increasing the survival of a male subject suffering from prostate cancer comprising administering to the subject a therapeutically effective amount of a compound or its pharmaceutically acceptable salt, or isomer, represented by a compound of formulas I-IX, IA-ID, IIA, IIB, VIIA, VIIB, VIIIA, VIIIB, IXA or IXB.

The invention provides a method of treating prostate cancer (PCa) or increasing the survival of a male subject suffering from prostate cancer comprising administering to the subject a therapeutically effective amount of a compound or its pharmaceutically acceptable salt, or isomer, represented by a compound of formulas 44-46, 98, 300-308, 1050-1064, and 1068.

The prostate cancer may be advanced prostate cancer, refractory prostate cancer, castration resistant prostate cancer (CRPC), metastatic CRPC (mCRPC), non-metastatic CRPC (nmCRPC), high-risk nmCRPC or any combination thereof.

The prostate cancer may depend on AR-FL and/or AR-SV for proliferation. The prostate or other cancer may be resistant to treatment with an androgen receptor antagonist. The prostate or other cancer may be resistant to treatment with enzalutamide, apalutamide, bicalutamide, abiraterone, ARN-509, ODM-201 (darolutamide), EPI-001, EPI-506, AZD-3514, galeterone, ASC-J9, flutamide, hydroxyflutamide, nilutamide, cyproterone acetate, ketoconazole, spironolactone, or any combination thereof. The method may also reduce the levels of AR, AR-FL, AR-FL with antiandrogen resistance-conferring AR-LBD mutations, AR-SV, gene-amplified AR, or any combination thereof.

In one embodiment, this invention provides a method of treating enzalutamide resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

In one embodiment, this invention provides a method of treating apalutamide resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

In one embodiment, this invention provides a method of treating abiraterone resistant prostate cancer comprising administering to the subject a therapeutically effective amount of a compound of this invention, or its optical isomer, isomer, pharmaceutically acceptable salt, pharmaceutical product, polymorph, hydrate or any combination thereof.

In one embodiment, this invention provides a method of treating triple negative breast cancer (TNBC) comprising administering to the subject a therapeutically effective amount of a compound of this invention, or its optical isomer, isomer, pharmaceutically acceptable salt, pharmaceutical product, polymorph, hydrate or any combination thereof.

The method may further comprise a second therapy such as androgen deprivation therapy (ADT) or LHRH agonist or antagonist. LHRH agonists include, but are not limited to, leuprolide acetate.

The invention encompasses a method of treating or inhibiting the progression of prostate cancer (PCa) or increasing the survival of a male subject suffering from prostate cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of prostate cancer (PCa) or increasing the survival of a male subject suffering from prostate cancer comprising administering to the subject a therapeutically effective amount of a SARD compound or pharmaceutically acceptable salt, wherein the compound is at least one of compounds 44-46, 98, 300-308, 1050-1064, and 1068.

The invention encompasses a method of treating or inhibiting the progression of refractory prostate cancer (PCa) or increasing the survival of a male subject suffering from refractory prostate cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of refractory prostate cancer (PCa) or increasing the survival of a male subject suffering from refractory prostate cancer comprising administering to the subject a therapeutically effective amount of a SARD compound or pharmaceutically acceptable salt, wherein the compound is represented by a compound of formulas 44-46, 98, 300-308, 1050-1064, and 1068.

The invention encompasses a method of treating or increasing the survival of a male subject suffering from castration resistant prostate cancer (CRPC) comprising administering to the subject a therapeutically effective amount of a SARD wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or increasing the survival of a male subject suffering from castration resistant prostate cancer (CRPC) comprising administering to the subject a therapeutically effective amount of a SARD wherein the compound is represented by a compound of formulas 44-46, 98, 300-308, 1050-1064, and 1068.

The method may further comprise administering androgen deprivation therapy to the subject.

The invention encompasses a method of treating or inhibiting the progression of enzalutamide resistant prostate cancer (PCa) or increasing the survival of a male subject suffering from enzalutamide resistant prostate cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of enzalutamide resistant prostate cancer (PCa) or increasing the survival of a male subject suffering from enzalutamide resistant prostate cancer comprising administering to the subject a therapeutically effective amount of a SARD compound or pharmaceutically acceptable salt, wherein the compound is represented by a compound of formulas 44-46, 98, 300-308, 1050-1064, and 1068.

The method may further comprise administering androgen deprivation therapy to the subject.

The invention encompasses a method of treating or inhibiting the progression of apalutamide resistant prostate cancer (PCa) or increasing the survival of a male subject suffering from apalutamide resistant prostate cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of apalutamide resistant prostate cancer (PCa) or increasing the survival of a male subject suffering from apalutamide resistant prostate cancer comprising administering to the subject a therapeutically effective amount of a SARD compound or pharmaceutically acceptable salt, wherein the compound is represented by a compound of formulas 44-46, 98, 300-308, 1050-1064, and 1068.

The invention encompasses a method of treating or inhibiting the progression of darolutamide resistant prostate cancer (PCa) or increasing the survival of a male subject suffering from darolutamide resistant prostate cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of darolutamide resistant prostate cancer (PCa) or increasing the survival of a male subject suffering from darolutamide resistant prostate cancer comprising administering to the subject a therapeutically effective amount of a SARD compound or pharmaceutically acceptable salt, wherein the compound is represented by a compound of formulas 44-46, 98, 300-308, 1050-1064, and 1068.

The method may further comprise administering androgen deprivation therapy to the subject.

The invention encompasses a method of treating or inhibiting the progression of triple negative breast cancer (TNBC) or increasing the survival of a female subject suffering from triple negative breast cancer comprising administering to the subject a therapeutically effective amount of a SARD compound or pharmaceutically acceptable salt, wherein the compound is represented by a compound of formulas I-IX, IA, IB, IC, ID, IIA, IIB, VIIA, VIIB, VIIIA, VIIIB, IXA or IXB, or any compounds as described herein.

The invention encompasses a method of treating or inhibiting the progression of triple negative breast cancer (TNBC) or increasing the survival of a female subject suffering from triple negative breast cancer comprising administering to the subject a therapeutically effective amount of a SARD compound or pharmaceutically acceptable salt, wherein the compound is represented by a compound of formulas 44-46, 98, 300-308, 1050-1064, and 1068.

The invention encompasses a method of treating breast cancer in a subject in need thereof, wherein said subject has AR expressing breast cancer, AR-SV expressing breast cancer, and/or AR-V7 expressing breast cancer, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating breast cancer in a subject in need thereof, wherein said subject has AR expressing breast cancer, AR-SV expressing breast cancer, and/or AR-V7 expressing breast cancer, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by a compound of formulas 44-46, 98, 300-308, 1050-1064, and 1068, or any compounds as described herein.

The invention encompasses a method of treating AR expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating AR expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by a compound of formulas 44-46, 98, 300-308, 1050-1064, and 1068, or any compounds as described herein.

The invention encompasses a method of treating AR-SV expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating AR-SV expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by a compound of formulas 44-46, 98, 300-308, 1050-1064, and 1068, or any compounds as described herein.

The invention encompasses a method of treating AR-V7 expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating AR-V7 expressing breast cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by a compound of formulas 44-46, 98, 300-308, 1050-1064, and 1068, or any compounds as described herein.

As used herein, the term “increase the survival” refers to a lengthening of time when describing the survival of a subject. Thus in this context, the compounds of the invention may be used to increase the survival of men with advanced prostate cancer, refractory prostate cancer, castration resistant prostate cancer (CRPC); metastatic CRPC (mCRPC); non-metastatic CRPC (nmCRPC); or high-risk nmCRPC; or women with TNBC.

Alternatively, as used herein, the terms “increase”, increasing”, or “increased” may be used interchangeably and refer to an entity becoming progressively greater (as in size, amount, number, or intensity), wherein for example the entity is sex hormone-binding globulin (SHBG) or prostate-specific antigen (PSA).

The compounds and compositions of the invention may be used for increasing metastasis-free survival (MFS) in a subject suffering from non-metastatic prostate cancer. The non-metastatic prostate cancer may be non-metastatic advanced prostate cancer, non-metastatic CRPC (nmCRPC), or high-risk nmCRPC.

The SARD compounds described herein may be used to provide a dual action. For example, the SARD compounds may treat prostate cancer and prevent metastasis. The prostate cancer may be refractory prostate cancer; advanced prostate cancer; castration resistant prostate cancer (CRPC); metastatic CRPC (mCRPC); non-metastatic CRPC (nmCRPC); or high-risk nmCRPC.

The SARD compounds described herein may be used to provide a dual action. For example, the SARD compounds may treat TNBC and prevent metastasis.

Men with advanced prostate cancer who are at high risk for progression to castration resistant prostate cancer (CRPC) are men on ADT with serum total testosterone concentrations greater than 20 ng/dL or men with advanced prostate cancer who at the time of starting ADT had either (1) confirmed Gleason pattern 4 or 5 prostate cancer, (2) metastatic prostate cancer, (3) a PSA doubling time<3 months, (4) a PSA≥20 ng/mL, or (5) a PSA relapse in <3 years after definitive local therapy (radical prostatectomy or radiation therapy).

Normal levels of prostate specific antigen (PSA) are dependent on several factors, such as age and the size of a male subject's prostate, among others. PSA levels in the range between 2.5-10 ng/mL are considered “borderline high” while levels above 10 ng/mL are considered “high.” A rate change or “PSA velocity” greater than 0.75/year is considered high. PSA levels may increase despite ongoing ADT or a history of ADT, surgical castration or despite treatment with antiandrogens and/or LHRH agonist.

Men with high risk non-metastatic castration resistant prostate cancer (high-risk nmCRPC) may include those with rapid PSA doubling times, having an expected progression-free survival of approximately 18 months or less (Miller K, Moul J W, Gleave M, et al. 2013. “Phase III, randomized, placebo-controlled study of once-daily oral zibotentan (ZD4054) in patients with non-metastatic castration-resistant prostate cancer,” Prostate Canc Prost Dis. February; 16:187-192). This relatively rapid progression of their disease underscores the importance of novel therapies for these individuals.

The methods of the invention may treat subjects with PSA levels greater than 8 ng/mL where the subject suffers from high-risk nmCRPC. The patient population includes subjects suffering from nmCRPC where PSA doubles in less than 8 months or less than 10 months. The method may also treat patient populations where the total serum testosterone levels are greater than 20 ng/mL in a subject suffering from high-risk nmCRPC. In one case, the serum free testosterone levels are greater than those observed in an orchiectomized male in a subject suffering from high-risk nmCRPC.

The pharmaceutical compositions of the invention may further comprise at least one LHRH agonist or antagonist, antiandrogen, anti-programmed death receptor 1 (anti-PD-1) drug or anti-PD-L1 drug. LHRH agonists include, but are not limited to, leuprolide acetate (Lupron®) (U.S. Pat. Nos. 5,480,656; 5,575,987; 5,631,020; 5,643,607; 5,716,640; 5,814,342; 6,036,976 hereby incorporated by reference) or goserelin acetate (Zoladex®) (U.S. Pat. Nos. 7,118,552; 7,220,247; 7,500,964 hereby incorporated by reference). LHRH antagonists include, but are not limited to, degarelix or abarelix. Antiandrogens include, but are not limited to, bicalutamide, flutamide, finasteride, dutasteride, enzalutamide, apalutamide, nilutamide, chlormadinone, abiraterone, or any combination thereof. Anti-PD-1 drugs include, but are not limited to, AMP-224, nivolumab, pembrolizumab, pidilizumab, and AMP-554. Anti-PD-L1 drugs include, but are not limited to, BMS-936559, atezolizumab, durvalumab, avelumab, and MPDL3280A. Anti-CTLA-4 drugs include, but are not limited to, ipilimumab and tremelimumab.

Treatment of prostate cancer, advanced prostate cancer, CRPC, mCRPC and/or nmCRPC may result in clinically meaningful improvement in prostate cancer related symptoms, function and/or survival. Clinically meaningful improvement can be determined by an increase in radiographic progression free survival (rPFS) if cancer is metastatic, or an increase metastasis-free survival (MFS) if cancer is non-metastatic, among others.

The invention encompasses methods of lowering serum prostate specific antigen (PSA) levels in a male subject suffering from prostate cancer, advanced prostate cancer, metastatic prostate cancer or castration resistant prostate cancer (CRPC) comprising administering a therapeutically effective amount of a SARD compound, wherein the compound is represented by the structure of formula I-IX, IA, IB, IC, ID, IIA, IIB, VIIA, VIIB, VIIIA, VIIIB, IXA or IXB.

The invention encompasses methods of lowering serum prostate specific antigen (PSA) levels in a male subject suffering from prostate cancer, advanced prostate cancer, metastatic prostate cancer or castration resistant prostate cancer (CRPC) comprising administering a therapeutically effective amount of a SARD compound, wherein the compound is represented by the structure of formulas 44-46, 98, 300-308, 1050-1064, and 1068.

The invention encompasses a method of secondary hormonal therapy that reduces serum PSA in a male subject suffering from castration resistant prostate cancer (CRPC) comprising administering a therapeutically effective amount of a compound of formula I-IX, IA, IB, IC, ID, IIA, IIB, VIIA, VIIB, VIIIA, VIIIB, IXA or IXB that reduces serum PSA in a male subject suffering from castration resistant prostate cancer.

The invention encompasses a method of secondary hormonal therapy that reduces serum PSA in a male subject suffering from castration resistant prostate cancer (CRPC) comprising administering a therapeutically effective amount of a compound of formulas 44-46, 98, 300-308, 1050-1064, and 1068 that reduces serum PSA in a male subject suffering from castration resistant prostate cancer.

The invention encompasses a method of reducing levels of AR, AR-full length (AR-FL), AR-FL with antiandrogen resistance-conferring AR-LBD mutations, AR-splice variant (AR-SV), and/or amplifications of the AR gene within the tumor in the subject in need thereof comprising administering a therapeutically effective amount of a compound of formula I-IX, IA, IB, IC, ID, IIA, IIB, VIIA, VIIB, VIIIA, VIIIB, IXA or IXB to reduce the level of AR, AR-full length (AR-FL), AR-FL with antiandrogen resistance-conferring AR-LBD or other AR mutations, AR-splice variant (AR-SV), and/or amplifications of the AR gene within the tumor.

The invention encompasses a method of reducing levels of AR, AR-full length (AR-FL), AR-FL with antiandrogen resistance-conferring AR-LBD mutations, AR-splice variant (AR-SV), and/or amplifications of the AR gene within the tumor in the subject in need thereof comprising administering a therapeutically effective amount of a compound of formulas 44-46, 98, 300-308, 1050-1064, and 1068 to reduce the level of AR, AR-full length (AR-FL), AR-FL with antiandrogen resistance-conferring AR-LBD or other AR mutations, AR-splice variant (AR-SV), and/or amplifications of the AR gene within the tumor. Alternatively, the invention encompasses a method of inhibiting an AR-axis that has been reactivated due to overexpression of GR.

The method may increase radiographic progression free survival (rPFS) or metastasis-free survival (MFS).

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, or non-metastatic castration resistant prostate cancer (nmCRPC) patient. In these subjects, the refractory may be enzalutamide resistant prostate cancer. In these subjects, the nmCRPC may be high-risk nmCRPC. Further the subject may be on androgen deprivation therapy (ADT) with or without castrate levels of total T.

Subjects may have non-metastatic cancer; failed androgen deprivation therapy (ADT), undergone orchidectomy, or have high or increasing prostate specific antigen (PSA) levels; subjects may be a patient with prostate cancer, advanced prostate cancer, refractory prostate cancer, CRPC patient, metastatic castration resistant prostate cancer (mCRPC) patient, or non-metastatic castration resistant prostate cancer (nmCRPC) patient. In these subjects, the refractory may be apalutamide resistant prostate cancer. In these subjects, the nmCRPC may be high-risk nmCRPC. Further the subject may be on androgen deprivation therapy (ADT) with or without castrate levels of total T.

As used herein, the phrase “a subject suffering from castration resistant prostate cancer” refers to a subject with at least one of the following characteristics: has been previously treated with androgen deprivation therapy (ADT); has responded to the ADT and currently has a serum PSA>2 ng/mL or >2 ng/mL and representing a 25% increase above the nadir achieved on the ADT; a subject which despite being maintained on androgen deprivation therapy is diagnosed to have serum PSA progression; a castrate level of serum total testosterone (<50 ng/dL) or a castrate level of serum total testosterone (<20 ng/dL). The subject may have rising serum PSA on two successive assessments at least 2 weeks apart; been effectively treated with ADT; or has a history of serum PSA response after initiation of ADT.

As used herein, the term “serum PSA progression” refers to a 25% or greater increase in serum PSA and an absolute increase of 2 ng/ml or more from the nadir; or to serum PSA>2 ng/mL, or >2 ng/mL and a 25% increase above the nadir after the initiation of androgen deprivation therapy (ADT). The term “nadir” refers to the lowest PSA level while a patient is undergoing ADT.

The term “serum PSA response” refers to at least one of the following: at least 90% reduction in serum PSA value prior to the initiation of ADT; to <10 ng/mL undetectable level of serum PSA (<0.2 ng/mL) at any time; at least 50% decline from baseline in serum PSA; at least 90% decline from baseline in serum PSA; at least 30% decline from baseline in serum PSA; or at least 10% decline from baseline in serum PSA.

The methods of this invention comprise administering a combination of forms of ADT and a compound of this invention. Forms of ADT include a LHRH agonist. LHRH agonist includes, but is not limited to, leuprolide acetate (Lupron®)(U.S. Pat. Nos. 5,480,656; 5,575,987; 5,631,020; 5,643,607; 5,716,640; 5,814,342; 6,036,976 hereby incorporated by reference) or goserelin acetate (Zoladex®) (U.S. Pat. Nos. 7,118,552; 7,220,247; 7,500,964 hereby incorporated by reference). Forms of ADT include, but are not limited to LHRH antagonists, reversible antiandrogens, or bilateral orchidectomy. LHRH antagonists include, but are not limited to, degarelix and abarelix. Antiandrogens include, but are not limited to, bicalutamide, flutamide, apalutamide, finasteride, dutasteride, enzalutamide, apalutamide, EPI-001, EPI-506, ARN-509, ODM-201 (darolutamide), nilutamide, chlormadinone, abiraterone, or any combination thereof.

The methods of the invention encompass administering at least one compound of the invention and a lyase inhibitor (e.g., abiraterone).

The term “advanced prostate cancer” refers to metastatic cancer having originated in the prostate, and having widely metastasized to beyond the prostate such as the surrounding tissues to include the seminal vesicles the pelvic lymph nodes or bone, or to other parts of the body. Prostate cancer pathologies are graded with a Gleason grading from 1 to 5 in order of increasing malignancy. Patients with significant risk of progressive disease and/or death from prostate cancer should be included in the definition and any patient with cancer outside the prostate capsule with disease stages as low as IIB clearly has “advanced” disease. “Advanced prostate cancer” can refer to locally advanced prostate cancer. Similarly, “advanced breast cancer” refers to metastatic cancer having originated in the breast, and having widely metastasized to beyond the breast to surrounding tissues or other parts of the body such as the liver, brain, lungs, or bone.

The term “refractory” may refer to cancers that do not respond to treatment. E.g., prostate or breast cancer may be resistant at the beginning of treatment or it may become resistant during treatment. “Refractory cancer” may also be referred to herein as “resistant cancer”.

The term “castration resistant prostate cancer” (CRPC) refers to advanced prostate cancer that is worsening or progressing while the patient remains on ADT or other therapies to reduce testosterone, or prostate cancer which is considered hormone refractory, hormone naïve, androgen independent or chemical or surgical castration resistant. CRPC may be the result of AR activation by intracrine androgen synthesis; expression of AR splice variants (AR-SV) that lack ligand binding domain (LBD); or expression of AR-LBD or other AR mutations with potential to resist antagonists. Castration resistant prostate cancer (CRPC) is an advanced prostate cancer which developed despite ongoing ADT and/or surgical castration. Castration resistant prostate cancer is defined as prostate cancer that continues to progress or worsen or adversely affect the health of the patient despite prior surgical castration, continued treatment with gonadotropin releasing hormone agonists (e.g., leuprolide) or antagonists (e.g., degarelix or abarelix), antiandrogens (e.g., bicalutamide, flutamide, apalutamide, enzalutamide, apalutamide, ketoconazole, aminoglutethamide), chemotherapeutic agents (e.g., docetaxel, paclitaxel, cabazitaxel, adriamycin, mitoxantrone, estramustine, cyclophosphamide), kinase inhibitors (imatinib (Gleevec®) or gefitinib (Iressa), cabozantinib (Cometriq™, also known as XL184)) or other prostate cancer therapies (e.g., vaccines (sipuleucel-T (Provenge), GVAX, etc.), herbal (PC-SPES) and lyase inhibitor (abiraterone)) as evidenced by increasing or higher serum levels of prostate specific antigen (PSA), metastasis, bone metastasis, pain, lymph node involvement, increasing size or serum markers for tumor growth, worsening diagnostic markers of prognosis, or patient condition.

Castration resistant prostate cancer may be defined as hormone naïve prostate cancer. In men with castration resistant prostate cancer, the tumor cells may have the ability to grow in the absence of androgens (hormones that promote the development and maintenance of male sex characteristics).

Many early prostate cancers require androgens for growth, but advanced prostate cancers are androgen-independent, or hormone naïve.

The term “androgen deprivation therapy” (ADT) may include orchiectomy; administering luteinizing hormone-releasing hormone (LHRH) analogs; administering luteinizing hormone-releasing hormone (LHRH) antagonists; administering 5(-reductase inhibitors; administering antiandrogens; administering inhibitors of testosterone biosynthesis; administering estrogens; or administering 17α-hydroxylase/C17,20 lyase (CYP17A1) inhibitors. LHRH drugs lower the amount of testosterone made by the testicles. Examples of LHRH analogs available in the United States include leuprolide (Lupron®, Viadur®, Eligard®), goserelin (Zoladex®), triptorelin (Trelstar®), and histrelin (Vantas®). Antiandrogens block the body's ability to use any androgens. Examples of antiandrogens drugs include darolutamide, enzalutamide (Xtandi®), apalutamide (Erleada®), flutamide (Eulexin®), apalutamide (Erleada®), bicalutamide (Casodex®), and nilutamide (Nilandron®). Luteinizing hormone-releasing hormone (LHRH) antagonists include abarelix (Plenaxis®) or degarelix (Firmagon®) (approved for use by the FDA in 2008 to treat advanced prostate cancer). 5α-Reductase inhibitors block the body's ability to convert testosterone to the more active androgen, 5α-dihydrotestosterone (DHT) and include drugs such as finasteride (Proscar®) and dutasteride (Avodart®). Inhibitors of testosterone biosynthesis include drugs such as ketoconazole (Nizoral®). Estrogens include diethylstilbestrol or 17β-estradiol. 17α-Hydroxylase/C17,20 lyase (CYP17A1) inhibitors include abiraterone (Zytiga®).

The invention encompasses a method of treating antiandrogen-resistant prostate cancer. The antiandrogen may include, but is not limited to, bicalutamide, hydroxyflutamide, flutamide, enzalutamide, apalutamide, or abiraterone.

The invention encompasses a method of treating prostate cancer in a subject in need thereof, wherein said subject has a rearranged AR, AR overexpressing prostate cancer, castration-resistant prostate cancer, castration-sensitive prostate cancer, AR-V7 expressing prostate cancer, or d567ES expressing prostate cancer, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating prostate cancer in a subject in need thereof, wherein said subject has a rearranged AR, AR overexpressing prostate cancer, castration-resistant prostate cancer, castration-sensitive prostate cancer, AR-V7 expressing prostate cancer, or d567ES expressing prostate cancer, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein.

In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or expressing castration-resistant prostate cancer.

In one embodiment, the castration-sensitive prostate cancer is F876L mutation expressing castration-sensitive prostate cancer, F876L_T877A double mutation castration-sensitive prostate cancer, and/expressing castration-sensitive prostate cancer.

In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating a rearranged AR and/or AR overexpressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating a rearranged AR and/or AR overexpressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein.

The invention encompasses a method of treating castration-resistant prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein. In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or expressing castration-resistant prostate cancer.

The invention encompasses a method of treating castration-resistant prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein. In one embodiment, the castration-resistant prostate cancer is a rearranged AR, AR overexpressing castration-resistant prostate cancer, F876L mutation expressing castration-resistant prostate cancer, F876L_T877A double mutation expressing castration-resistant prostate cancer, AR-V7 expressing castration-resistant prostate cancer, d567ES expressing castration-resistant prostate cancer, and/or expressing castration-resistant prostate cancer.

The invention encompasses a method of treating castration-sensitive prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein. In one embodiment, the castration-sensitive prostate cancer is F876L mutation expressing castration-sensitive prostate cancer, F876L_T877A double mutation castration-sensitive prostate cancer, and/or expressing castration-sensitive prostate cancer. In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating castration-sensitive prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein. In one embodiment, the castration-sensitive prostate cancer is F876L mutation expressing castration-sensitive prostate cancer, F876L_T877A double mutation castration-sensitive prostate cancer, and/or expressing castration-sensitive prostate cancer. In one embodiment, the treating of castration-sensitive prostate cancer is conducted in a non-castrate setting, or as monotherapy, or when castration-sensitive prostate cancer tumor is resistant to darolutamide, enzalutamide, apalutamide, and/or abiraterone.

The invention encompasses a method of treating AR-V7 expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating AR-V7 expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein.

The invention encompasses a method of treating d567ES expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein.

The invention encompasses a method of treating d567ES expressing prostate cancer in a subject in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented represented by a compound of formulas 44-46, 98, 300-308, 1050-1064, and 1068, or any compounds as described herein.

### Treatment of Triple Negative Breast Cancer (TNBC)

Triple negative breast cancer (TNBC) is a type of breast cancer lacking the expression of the estrogen receptor (ER), progesterone receptor (PR), and HER2 receptor kinase. As such, TNBC lacks the hormone and kinase therapeutic targets used to treat other types of primary breast cancers. Correspondingly, chemotherapy is often the initial pharmacotherapy for TNBC. Interestingly, AR is often still expressed in TNBC and may offer a hormone targeted therapeutic alternative to chemotherapy. In ER-positive breast cancer, AR is a positive prognostic indicator as it is believed that activation of AR limits and/or opposes the effects of the ER in breast tissue and tumors. However, in the absence of ER, it is possible that AR actually supports the growth of breast cancer tumors. Though the role of AR is not fully understood in TNBC, there is evidence that certain TNBC's may be supported by androgen independent activation of AR-SVs lacking the LBD or androgen-dependent activation of AR full length. As such, enzalutamide, apalutamide and other LBD-directed traditional AR antagonists would not be able to antagonize AR-SVs in these TNBC's. However, SARDs of this invention which are capable of destroying AR-SVs (see Table 1 and Examples 2 and 7) through a binding site in the NTD of AR (see Example 9 of US2017-0368003) would be able to antagonize AR in these TNBC's and provide an anti-tumor effect, as shown in Example 8 of US2017-0368003.

### Treatment of Kennedy's Disease

Muscle atrophy (MA) is characterized by wasting away or diminution of muscle and a decrease in muscle mass. For example, post-polio MA is muscle wasting that occurs as part of the post-polio syndrome (PPS). The atrophy includes weakness, muscle fatigue, and pain. Another type of MA is X-linked spinal-bulbar muscular atrophy (SBMA—also known as Kennedy's Disease). This disease arises from a defect in the androgen receptor gene on the X chromosome, affects only males, and its onset is in late adolescence to adulthood. Proximal limb and bulbar muscle weakness results in physical limitations including dependence on a wheelchair in some cases. The mutation results in an extended polyglutamine tract at the N-terminal domain of the androgen receptor (polyQ AR).

Binding and activation of the polyQ AR by endogeneous androgens (testosterone and DHT) results in unfolding and nuclear translocation of the mutant androgen receptor. The androgen-induced toxicity and androgen-dependent nuclear accumulation of polyQ AR protein seems to be central to the pathogenesis. Therefore, the inhibition of the androgen-activated polyQ AR might be a therapeutic option (A. Baniahmad. Inhibition of the androgen receptor by antiandrogens in spinobulbar muscle atrophy. J. Mol. Neurosci. 2016 58(3), 343-347). These steps are required for pathogenesis and result in partial loss of transactivation function (i.e., an androgen insensitivity) and a poorly understood neuromuscular degeneration. Peripheral polyQ AR anti-sense therapy rescues disease in mouse models of SBMA (Cell Reports 7, 774-784, May 8, 2014). Further support of use antiandrogen comes in a report in which the antiandrogen flutamide protects male mice from androgen-dependent toxicity in three models of spinal bulbar muscular atrophy (Renier K J, Troxell-Smith S M, Johansen J A, Katsuno M, Adachi H, Sobue G, Chua J P, Sun Kim H, Lieberman A P, Breedlove S M, Jordan C L. Endocrinology 2014, 155(7), 2624-2634). These steps are required for pathogenesis and result in partial loss of transactivation function (i.e., an androgen insensitivity) and a poorly understood neuromuscular degeneration. Currently there are no disease-modifying treatments but rather only symptom directed treatments. Efforts to target the polyQ AR as the proximal mediator of toxicity by harnessing cellular machinery to promote its degradation hold promise for therapeutic intervention.

Selective androgen receptor degraders such as those reported herein bind to, inhibit transactivation, and degrade all androgen receptors tested to date (full length, splice variant, antiandrogen resistance mutants, etc.), indicating that they are promising leads for treatment diseases whose pathogenesis is androgen-dependent such as SBMA.

The invention encompasses methods of treating Kennedy's disease comprising administering a therapeutically effective amount of a compound of formulas I-IX, IA, IB, IC, ID, IIA, IIB, VIIA, VIIB, VIIIA, VIIIB, IXA or IXB.

The invention encompasses methods of treating Kennedy's disease comprising administering a therapeutically effective amount of a compound of formulas 44-46, 98, 300-308, 1050-1064, and 1068.

As used herein, the term “androgen receptor associated conditions” or “androgen sensitive diseases or disorders” or “androgen-dependent diseases or disorders” are conditions, diseases, or disorders that are modulated by or whose pathogenesis is dependent upon the activity of the androgen receptor. The androgen receptor is expressed in most tissues of the body however it is overexpressed in, inter alia, the prostate and skin. ADT has been the mainstay of prostate cancer treatment for many years, and SARDs may also be useful in treating various prostate cancers, benign prostatic hypertrophy, prostamegaly, and other maladies of the prostate.

The invention encompasses methods of treating benign prostatic hypertrophy comprising administering a therapeutically effective amount of at least one compound of formulas I-IX, IA, IB, IC, ID, IIA, IIB, VIIA, VIIB, VIIIA, VIIIB, IXA or IXB.

The invention encompasses methods of treating benign prostatic hypertrophy comprising administering a therapeutically effective amount of at least one compound of formulas 44-46, 98, 300-308, 1050-1064, and 1068.

The invention encompasses methods of treating prostamegaly comprising administering a therapeutically effective amount of at least one compound of formulas I-IX, IA, IB, IC, ID, IIA, IIB, VIIA, VIIB, VIIIA, VIIIB, IXA or IXB.

The invention encompasses methods of treating prostamegaly comprising administering a therapeutically effective amount of at least one compound of formulas 44-46, 98, 300-308, 1050-1064, and 1068.

The invention encompasses methods of treating hyperproliferative prostatic disorders and diseases comprising administering a therapeutically effective amount of a compound of formulas I-IX, IA, IB, IC, ID, IIA, IIB, VIIA, VIIB, VIIIA, VIIIB, IXA or IXB.

The invention encompasses methods of treating hyperproliferative prostatic disorders and diseases comprising administering a therapeutically effective amount of a compound of formulas 44-46, 98, 300-308, 1050-1064, and 1068.

The effect of the AR on the skin is apparent in the gender dimorphism and puberty related dermatological problems common to teens and early adults. The hyperandrogenism of puberty stimulates terminal hair growth, sebum production, and predisposes male teens to acne, acne vulgaris, seborrhea, excess sebum, hidradenitis suppurativa, hirsutism, hypertrichosis, hyperpilosity, androgenic alopecia, male pattern baldness, and other dermatological maladies. Although antiandrogens theoretically should prevent the hyperandrogenic dermatological diseases discussed, they are limited by toxicities, sexual side effects, and lack of efficacy when topically applied. The SARDs of this invention potently inhibit ligand-dependent and ligand-independent AR activation, and (in some cases) have short biological half-lives in the serum, suggesting that topically formulated SARDs of this invention could be applied to the areas affected by acne, seborrheic dermatitis, and/or hirsutism without risk of systemic side effects.

The invention encompasses methods of treating acne, acne vulgaris, seborrhea, seborrheic dermatitis, hidradenitis supporativa, hirsutism, hypertrichosis, hyperpilosity, or alopecia comprising administering a therapeutically effective amount of a compound of formulas I-IX, IA, IB, IC, ID, IIA, IIB, VIIA, VIIB, VIIIA, VIIIB, IXA or IXB, or any compounds as described herein.

The invention encompasses methods of treating acne, acne vulgaris, seborrhea, seborrheic dermatitis, hidradenitis supporativa, hirsutism, hypertrichosis, hyperpilosity, or alopecia comprising administering a therapeutically effective amount of a compound of formulas 44-46, 98, 300-308, 1050-1064, and 1068.

The compounds and/or compositions described herein may be used for treating hair loss, alopecia, androgenic alopecia, alopecia areata, alopecia secondary to chemotherapy, alopecia secondary to radiation therapy, alopecia induced by scarring or alopecia induced by stress. Generally “hair loss” or “alopecia” refers to baldness as in the very common type of male-pattern baldness. Baldness typically begins with patch hair loss on the scalp and sometimes progresses to complete baldness and even loss of body hair. Hair loss affects both males and females.

The invention encompasses methods of treating androgenic alopecia comprising administering a therapeutically effective amount of a compound of formula I-IX, IA, IB, IC, ID, IIA, IIB, VIIA, VIIB, VIIIA, VIIIB, IXA or IXB, or any compounds as described herein.

The invention encompasses methods of treating androgenic alopecia comprising administering a therapeutically effective amount of a compound of formulas 44-46, 98, 300-308, 1050-1064, and 1068.

SARDs of this invention may also be useful in the treatment of hormonal conditions in females which can have hyperandrogenic pathogenesis such as precocious puberty, early puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, and/or vaginal dryness.

The invention encompasses methods of treating precocious puberty or early puberty, dysmenorrhea or amenorrhea, multilocular uterus syndrome, endometriosis, hysteromyoma, abnormal uterine bleeding, hyper-androgenic diseases (such as polycystic ovary syndrome (PCOS)), fibrocystic breast disease, fibroids of the uterus, ovarian cysts, polycystic ovary syndrome, pre-eclampsia, eclampsia of pregnancy, preterm labor, premenstrual syndrome, or vaginal dryness comprising administering a therapeutically effective amount of a compound of formulas I-IX, IA, IB, IC, ID, IIA, IIB, VIIA, VIIB, VIIIA, VIIIB, IXA or IXB, or any compounds as described herein.

The invention encompasses methods of treating precocious puberty or early puberty, dysmenorrhea or amenorrhea, multilocular uterus syndrome, endometriosis, hysteromyoma, abnormal uterine bleeding, hyper-androgenic diseases (such as polycystic ovary syndrome (PCOS)), fibrocystic breast disease, fibroids of the uterus, ovarian cysts, polycystic ovary syndrome, pre-eclampsia, eclampsia of pregnancy, preterm labor, premenstrual syndrome, or vaginal dryness comprising administering a therapeutically effective amount of a compound of formulas 44-46, 98, 300-308, 1050-1064, and 1068.

The invention encompasses methods of treating, suppressing, reducing the incidence, reducing the severity, or inhibiting the progression of a hormonal condition in a male in need thereof, comprising administering to the subject a therapeutically effective amount of a selective androgen receptor degrader (SARD) compound, or its isomer, pharmaceutically acceptable salt, pharmaceutical product, polymorph, hydrate or any combination thereof, wherein said SARD compound is represented by the structure of formula I-IX, IA, IB, IC, ID, IIA, IIB, VIIA, VIIB, VIIIA, VIIIB, IXA or IXB, or any compounds as described herein. In one embodiment, the condition is sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer. In one embodiment, the hormonal condition includes, but is not limited to, hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers.

The invention encompasses methods of treating, suppressing, reducing the incidence, reducing the severity, or inhibiting the progression of a hormonal condition in a male in need thereof, comprising administering a therapeutically effective amount of a compound of formulas 44-46, 98, 300-308, 1050-1064, and 1068, or its isomer, pharmaceutically acceptable salt, pharmaceutical product, polymorph, hydrate or any combination thereof. In one embodiment, the hormonal condition includes, but is not limited to, hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers. In one embodiment, the condition is sexual dysfunction, decreased sexual libido, erectile dysfunction, hypogonadism, sarcopenia, osteopenia, osteoporosis, alterations in cognition and mood, depression, anemia, hair loss, obesity, benign prostate hyperplasia and/or prostate cancer.

SARDs of this invention may also find utility in treatment of sexual perversion, hypersexuality, paraphilias, androgen psychosis, virilization, androgen insensitivity syndromes (AIS) (such as complete AIS (CAIS) and partial AIS (PAIS)), and improving ovulation in an animal.

The invention encompasses methods of treating sexual perversion, hypersexuality, paraphilias, androgen psychosis, virilization androgen, insensitivity syndromes, increasing or modulating or improving ovulation comprising administering a therapeutically effective amount of a compound of formulas I-IX, IA, IB, IC, ID, IIA, IIB, VIIA, VIIB, VIIIA, VIIIB, IXA or IXB, or any compounds as described herein.

The invention encompasses methods of treating sexual perversion, hypersexuality, paraphilias, androgen psychosis, virilization androgen, insensitivity syndromes, increasing or modulating or improving ovulation comprising administering a therapeutically effective amount of a compound of formulas 44-46, 98, 300-308, 1050-1064, and 1068.

SARDs of this invention may also be useful for treating hormone-dependent cancers such as prostate cancer, breast cancer, testicular cancer, ovarian cancer, hepatocellular carcinoma, urogenital cancer, etc. In another embodiment, the breast cancer is triple negative breast cancer. Further, local or systemic SARD administration may be useful for treatment of precursors of hormone-dependent cancers such as prostatic intraepithelial neoplasia (PIN) and atypical small acinar proliferation (ASAP).

The invention encompasses methods of treating breast cancer, testicular cancer, uterine cancer, ovarian cancer, urogenital cancer, precursors of prostate cancer, or AR related or AR expressing solid tumors, comprising administering a therapeutically effective amount of a compound of formulas I-IX, IA, IB, IC, ID, IIA, IIB, VIIA, VIIB, VIIIA, VIIIB, IXA or IXB. A precursor of prostate cancers may be prostatic intraepithelial neoplasia (PIN) or atypical small acinar proliferation (ASAP). The tumor may be hepatocellular carcinoma (HCC) or bladder cancer. Serum testosterone may be positively linked to the development of HCC. Based on epidemiologic, experimental observations, and notably the fact that men have a substantially higher risk of bladder cancer than women, androgens and/or the AR may also play a role in bladder cancer initiation. 25 [00277] The invention encompasses methods of treating breast cancer, testicular cancer, uterine cancer, ovarian cancer, urogenital cancer, precursors of prostate cancer, or AR related or AR expressing solid tumors, comprising administering a therapeutically effective amount of a compound of formulas 44-46, 98, 300-308, 1050-1064, and 1068. A precursor of prostate cancers may be prostatic intraepithelial neoplasia (PIN) or atypical small acinar proliferation (ASAP). The tumor may be hepatocellular carcinoma (HCC) or bladder cancer. Serum testosterone may be positively linked to the development of HCC. Based on epidemiologic, experimental observations, and notably the fact that men have a substantially higher risk of bladder cancer than women, androgens and/or the AR may also play a role in bladder cancer initiation.

Although traditional antiandrogens such as enzalutamide, apalutamide, bicalutamide and flutamide and androgen deprivation therapies (ADT) such as leuprolide were approved for use in prostate cancer, there is significant evidence that antiandrogens could also be used in a variety of other hormone-dependent and hormone-independent cancers. For example, antiandrogens have been successfully tested in breast cancer (enzalutamide; Breast Cancer Res (2014) 16(1): R7), non-small cell lung cancer (shRNAi AR), renal cell carcinoma (ASC-J9), partial androgen insensitivity associated malignancies such as gonadal tumors and seminoma, advanced pancreatic cancer (World J Gastroenterology 20(29):9229), cancer of the ovary, fallopian tubes, or peritoneum, cancer of the salivary gland (Head and Neck (2016) 38: 724-731; ADT was tested in AR-expressing recurrent/metastatic salivary gland cancers and was confirmed to have benefit on progression free survival and overall survival endpoints), bladder cancer (Oncotarget 6 (30): 29860-29876); Int J Endocrinol (2015), Article ID 384860), pancreatic cancer, lymphoma (including mantle cell), and hepatocellular carcinoma. Use of a more potent antiandrogen such as a SARD in these cancers may treat the progression of these and other cancers. Other cancers may also benefit from SARD treatment such as testicular cancer, uterine cancer, ovarian cancer, urogenital cancer, breast cancer, brain cancer, skin cancer, lymphoma, liver cancer, renal cancer, osteosarcoma, pancreatic cancer, endometrial cancer, lung cancer, non-small cell lung cancer (NSCLC), colon cancer, perianal adenoma, or central nervous system cancer.

SARDs of this invention may also be useful for treating other cancers containing AR such as breast, brain, skin, ovarian, bladder, lymphoma, liver, kidney, pancreas, endometrium, lung (e.g., NSCLC), colon, perianal adenoma, osteosarcoma, CNS, melanoma, hypercalcemia of malignancy and metastatic bone disease, etc.

Thus, the invention encompasses methods of treating hypercalcemia of malignancy, metastatic bone disease, brain cancer, skin cancer, bladder cancer, lymphoma, liver cancer, renal cancer, osteosarcoma, pancreatic cancer, endometrial cancer, lung cancer, central nervous system cancer, gastric cancer, colon cancer, melanoma, amyotrophic lateral sclerosis (ALS), and/or uterine fibroids comprising administering a therapeutically effective amount of a compound of formulas I-IX, IA, IB, IC, ID, IIA, IIB, VIIA, VIIB, VIIIA, VIIIB, IXA or IXB, or any compounds as described herein. The lung cancer may be non-small cell lung cancer (NSCLC).

The invention encompasses methods of treating hypercalcemia of malignancy, metastatic bone disease, brain cancer, skin cancer, bladder cancer, lymphoma, liver cancer, renal cancer, osteosarcoma, pancreatic cancer, endometrial cancer, lung cancer, central nervous system cancer, gastric cancer, colon cancer, melanoma, amyotrophic lateral sclerosis (ALS), and/or uterine fibroids comprising administering a therapeutically effective amount of a compound of formulas 44-46, 98, 300-308, 1050-1064, and 1068. The lung cancer may be non-small cell lung cancer (NSCLC).

SARDs of this invention may also be useful for the treating of non-hormone-dependent cancers. Non-hormone-dependent cancers include liver, salivary duct, etc.

In another embodiment, the SARDs of this invention are used for treating gastric cancer. In another embodiment, the SARDs of this invention are used for treating salivary duct carcinoma. In another embodiment, the SARDs of this invention are used for treating bladder cancer. In another embodiment, the SARDs of this invention are used for treating esophageal cancer. In another embodiment, the SARDs of this invention are used for treating pancreatic cancer. In another embodiment, the SARDs of this invention are used for treating colon cancer. In another embodiment, the SARDs of this invention are used for treating non-small cell lung cancer. In another embodiment, the SARDs of this invention are used for treating renal cell carcinoma.

AR plays a role in cancer initiation in hepatocellular carcinoma (HCC). Therefore, targeting AR may be an appropriate treatment for patients with early stage HCC. In late-stage HCC disease, there is evidence that metastasis is suppressed by androgens. In another embodiment, the SARDs of this invention are used for treating hepatocellular carcinoma (HCC).

Locati et al. in Head & Neck, 2016, 724-731 demonstrated the use of androgen deprivation therapy (ADT) in AR-expressing recurrent/metastatic salivary gland cancers and confirmed improved progression free survival and overall survival endpoints with ADT. In another embodiment, the SARDs of this invention are used for treating salivary gland cancer.

Kawahara et al. in Oncotarget, 2015, Vol 6 (30), 29860-29876 demonstrated that ELK1 inhibition, together with AR inactivation, has the potential of being a therapeutic approach for bladder cancer. McBeth et al. Int J Endocrinology, 2015, Vol 2015, Article ID 384860 suggested that the combination of antiandrogen therapy plus glucocorticoids as treatment of bladder cancer as this cancer is believed to have an inflammatory etiology. In another embodiment, the SARDs of this invention are used for treating bladder cancer, optionally in combination with glucocorticoids.

### Abdominal Aortic Aneurysm (AAA)

An abdominal aortic aneurysm (AAA) is an enlarged area in the lower part of the aorta, the major blood vessel that supplies blood to the body. The aorta, about the thickness of a garden hose, runs from your heart through the center of your chest and abdomen. Because the aorta is the body's main supplier of blood, a ruptured abdominal aortic aneurysm can cause life-threatening bleeding. Depending on the size and the rate at which your abdominal aortic aneurysm is growing, treatment may vary from watchful waiting to emergency surgery. Once an abdominal aortic aneurysm is found, doctors will closely monitor it so that surgery can be planned if it is necessary. Emergency surgery for a ruptured abdominal aortic aneurysm can be risky. AR blockade (pharmacologic or genetic) reduces AAA. Davis et al. (Davis J P, Salmon M, Pope N H, Lu G, Su G, Meher A, Ailawadi G, Upchurch G R Jr. J Vase Surg (2016) 63(6):1602-1612) showed that flutamide (50 mg/kg) or ketoconazole (150 mg/kg) attenuated AAA induced by porcine pancreatic elastase (0.35 U/mL) by 84.2% and 91.5% compared to vehicle (121%). Further AR −/− mice showed attenuated AAA growth (64.4%) compared to wildtype (both treated with elastase). Correspondingly, administration of a SARD to a patient suffering from an AAA may help reverse, treat or delay progression of AAA to the point where surgery is needed.

### Treatment of Wounds

Wounds and/or ulcers are normally found protruding from the skin or on a mucosal surface or as a result of an infarction in an organ. A wound may be a result of a soft tissue defect or a lesion or of an underlying condition. The term “wound” denotes a bodily injury with disruption of the normal integrity of tissue structures, sore, lesion, necrosis, and/or ulcer. The term “sore” refers to any lesion of the skin or mucous membranes and the term “ulcer” refers to a local defect, or excavation, of the surface of an organ or tissue, which is produced by the sloughing of necrotic tissue. “Lesion” generally includes any tissue defect. “Necrosis” refers to dead tissue resulting from infection, injury, inflammation, or infarctions. All of these are encompassed by the term “wound,” which denotes any wound at any particular stage in the healing process including the stage before any healing has initiated or even before a specific wound like a surgical incision is made (prophylactic treatment).

Examples of wounds which can be treated in accordance with the present invention are aseptic wounds, contused wounds, incised wounds, lacerated wounds, non-penetrating wounds (i.e. wounds in which there is no disruption of the skin but there is injury to underlying structures), open wounds, penetrating wounds, perforating wounds, puncture wounds, septic wounds, subcutaneous wounds, etc. Examples of sores include, but are not limited to, bed sores, canker sores, chrome sores, cold sores, pressure sores, etc. Examples of ulcers include, but are not limited to, peptic ulcer, duodenal ulcer, gastric ulcer, gouty ulcer, diabetic ulcer, hypertensive ischemic ulcer, stasis ulcer, ulcus cruris (venous ulcer), sublingual ulcer, submucous ulcer, symptomatic ulcer, trophic ulcer, tropical ulcer, veneral ulcer, e.g., caused by gonorrhoea (including urethritis, endocervicitis and proctitis). Conditions related to wounds or sores which may be successfully treated according to the invention include, but are not limited to, burns, anthrax, tetanus, gas gangrene, scalatina, erysipelas, sycosis barbae, folliculitis, impetigo contagiosa, impetigo bullosa, etc. It is understood, that there may be an overlap between the use of the terms “wound” and “ulcer,” or “wound” and “sore” and, furthermore, the terms are often used at random.

The kinds of wounds to be treated according to the invention include also: i) general wounds such as, e.g., surgical, traumatic, infectious, ischemic, thermal, chemical and bullous wounds; ii) wounds specific for the oral cavity such as, e.g., post-extraction wounds, endodontic wounds especially in connection with treatment of cysts and abscesses, ulcers and lesions of bacterial, viral or autoimmunological origin, mechanical, chemical, thermal, infectious and lichenoid wounds; herpes ulcers, stomatitis aphthosa, acute necrotising ulcerative gingivitis and burning mouth syndrome are specific examples; and iii) wounds on the skin such as, e.g., neoplasm, burns (e.g. chemical, thermal), lesions (bacterial, viral, autoimmunological), bites and surgical incisions. Another way of classifying wounds is by tissue loss, where: i) small tissue loss (due to surgical incisions, minor abrasions, and minor bites) or ii) significant tissue loss. The latter group includes ischemic ulcers, pressure sores, fistulae, lacerations, severe bites, thermal burns and donor site wounds (in soft and hard tissues) and infarctions. Other wounds include ischemic ulcers, pressure sores, fistulae, severe bites, thermal burns, or donor site wounds.

Ischemic ulcers and pressure sores are wounds, which normally only heal very slowly and especially in such cases an improved and more rapid healing is of great importance to the patient. Furthermore, the costs involved in the treatment of patients suffering from such wounds are markedly reduced when the healing is improved and takes place more rapidly.

Donor site wounds are wounds which e.g. occur in connection with removal of hard tissue from one part of the body to another part of the body e.g. in connection with transplantation. The wounds resulting from such operations are very painful and an improved healing is therefore most valuable.

In one case, the wound to be treated is selected from the group consisting of aseptic wounds, infarctions, contused wounds, incised wounds, lacerated wounds, non-penetrating wounds, open wounds, penetrating wounds, perforating wounds, puncture wounds, septic wounds, and subcutaneous wounds.

The invention encompasses methods of treating a subject suffering from a wound comprising administering to the subject a therapeutically effective amount of a compound of formulas I-IX, IA, IB, IC, ID, IIA, IIB, VIIA, VIIB, VIIIA, VIIIB, IXA or IXB, pharmaceutically acceptable salt thereof, or a pharmaceutical composition thereof.

The invention encompasses methods of treating a subject suffering from a wound comprising administering to the subject a therapeutically effective amount of a compound of formulas 44-46, 98, 300-308, 1050-1064, and 1068, pharmaceutically acceptable salt thereof, or a pharmaceutical composition thereof.

The invention encompasses methods of treating a subject suffering from a burn comprising administering to the subject a therapeutically effective amount of a compound of formulas I-IX, IA, IB, IC, ID, IIA, IIB, VIIA, VIIB, VIIIA, VIIIB, IXA or IXB, pharmaceutically acceptable salt thereof, or a pharmaceutical composition thereof.

The invention encompasses methods of treating a subject suffering from a burn comprising administering to the subject a therapeutically effective amount of a compound of formulas 44-46, 98, 300-308, 1050-1064, and 1068, pharmaceutically acceptable salt thereof, or a pharmaceutical composition thereof.

The term “skin” is used in a very broad sense embracing the epidermal layer of the skin and in those cases where the skin surface is more or less injured also the dermal layer of the skin. Apart from the stratum corneum, the epidermal layer of the skin is the outer (epithelial) layer and the deeper connective tissue layer of the skin is called the dermis.

Since the skin is the most exposed part of the body, it is particularly susceptible to various kinds of injuries such as, e.g., ruptures, cuts, abrasions, burns and frostbites or injuries arising from various diseases. Furthermore, much skin is often destroyed in accidents. However, due to the important barrier and physiologic function of the skin, the integrity of the skin is important to the well-being of the individual, and any breach or rupture represents a threat that must be met by the body in order to protect its continued existence.

Apart from injuries on the skin, injuries may also be present in all kinds of tissues (i.e. soft and hard tissues). Injuries on soft tissues including mucosal membranes and/or skin are especially relevant in connection with the present invention.

Healing of a wound on the skin or on a mucosal membrane undergoes a series of stages that results either in repair or regeneration of the skin or mucosal membrane. In recent years, regeneration and repair have been distinguished as the two types of healing that may occur. Regeneration may be defined as a biological process whereby the architecture and function of lost tissue are completely renewed. Repair, on the other hand, is a biological process whereby continuity of disrupted tissue is restored by new tissues which do not replicate the structure and function of the lost ones.

The majority of wounds heal through repair, meaning that the new tissue formed is structurally and chemically unlike the original tissue (scar tissue). In the early stage of the tissue repair, one process which is almost always involved is the formation of a transient connective tissue in the area of tissue injury. This process starts by formation of a new extracellular collagen matrix by fibroblasts. This new extracellular collagen matrix is then the support for a connective tissue during the final healing process. The final healing is, in most tissues, a scar formation containing connective tissue. In tissues which have regenerative properties, such as, e.g., skin and bone, the final healing includes regeneration of the original tissue. This regenerated tissue has frequently also some scar characteristics, e.g. a thickening of a healed bone fracture.

Under normal circumstances, the body provides mechanisms for healing injured skin or mucosa in order to restore the integrity of the skin barrier or the mucosa. The repair process for even minor ruptures or wounds may take a period of time extending from hours and days to weeks. However, in ulceration, the healing can be very slow and the wound may persist for an extended period of time, i.e. months or even years.

Burns are associated with reduced testosterone levels, and hypogonadism is associated with delayed wound healing. The invention encompasses methods for treating a subject suffering from a wound or a burn by administering at least one SARD compound according to this invention. The SARD may promote resolving of the burn or wound, participates in the healing process of a burn or a wound, or, treats a secondary complication of a burn or wound.

The treatment of burns or wounds may further use at least one growth factor such as epidermal growth factor (EGF), transforming growth factor-α (TGF-α), platelet derived growth factor (PDGF), fibroblast growth factors (FGFs) including acidic fibroblast growth factor (α-FGF) and basic fibroblast growth factor (β-FGF), transforming growth factor-β (TGF-β) and insulin like growth factors (IGF-1 and IGF-2), or any combination thereof, which promote wound healing.

Wound healing may be measured by many procedures known in the art, including, but not limited to, wound tensile strength, hydroxyproline or collagen content, procollagen expression, or re-epithelialization. As an example, a SARD as described herein may be administered orally or topically at a dosage of about 0.1-100 mg per day. Therapeutic effectiveness is measured as effectiveness in enhancing wound healing as compared to the absence of the SARD compound. Enhanced wound healing may be measured by known techniques such as decrease in healing time, increase in collagen density, increase in hydroxyproline, reduction in complications, increase in tensile strength, and increased cellularity of scar tissue.

The term “reducing the pathogenesis” is to be understood to encompass reducing tissue damage, or organ damage associated with a particular disease, disorder or condition. The term may include reducing the incidence or severity of an associated disease, disorder or condition, with that in question or reducing the number of associated diseases, disorders or conditions with the indicated, or symptoms associated thereto.

### Pharmaceutical Compositions

The compounds of the invention may be used in pharmaceutical compositions. As used herein, “pharmaceutical composition” means either the compound or pharmaceutically acceptable salt of the active ingredient with a pharmaceutically acceptable carrier or diluent. A “therapeutically effective amount” as used herein refers to that amount which provides a therapeutic effect for a given indication and administration regimen.

As used herein, the term “administering” refers to bringing a subject in contact with a compound of the present invention. As used herein, administration can be accomplished in vitro, i.e. in a test tube, or in vivo, i.e. in cells or tissues of living organisms, for example humans. The subjects may be a male or female subject or both.

Numerous standard references are available that describe procedures for preparing various compositions or formulations suitable for administration of the compounds of the invention. Examples of methods of making formulations and preparations can be found in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

The mode of administration and dosage form are closely related to the therapeutic amounts of the compounds or compositions which are desirable and efficacious for the given treatment application.

The pharmaceutical compositions of the invention can be administered to a subject by any method known to a person skilled in the art. These methods include, but are not limited to, orally, parenterally, intravascularly, paracancerally, transmucosally, transdermally, intramuscularly, intranasally, intravenously, intradermally, subcutaneously, sublingually, intraperitoneally, intraventricularly, intracranially, intravaginally, by inhalation, rectally, or intratumorally. These methods include any means in which the composition can be delivered to tissue (e.g., needle or catheter). Alternatively, a topical administration may be desired for application to dermal, ocular, or mucosal surfaces. Another method of administration is via aspiration or aerosol formulation. The pharmaceutical compositions may be administered topically to body surfaces, and are thus formulated in a form suitable for topical administration. Suitable topical formulations include gels, ointments, creams, lotions, drops and the like. For topical administrations, the compositions are prepared and applied as solutions, suspensions, or emulsions in a physiologically acceptable diluent with or without a pharmaceutical carrier.

Suitable dosage forms include, but are not limited to, oral, rectal, sub-lingual, mucosal, nasal, ophthalmic, subcutaneous, intramuscular, intravenous, transdermal, spinal, intrathecal, intra-articular, intra-arterial, sub-arachinoid, bronchial, lymphatic, and intra-uterile administration, and other dosage forms for systemic delivery of active ingredients. Depending on the indication, formulations suitable for oral or topical administration are preferred.

Topical Administration: The compounds of formulas I-IX, IA, IB, IC, ID, IIA, IIB, VIIA, VIIB, VIIIA, VIIIB, IXA or IXB may be administered topically. As used herein, “topical administration” refers to application of the compounds of formulas I-IX, IA, IB, IC, ID, IIA, IIB, VIIA, VIIB, VIIIA, VIIIB, IXA or IXB (and optional carrier) directly to the skin and/or hair.

The compounds of formulas 44-46, 98, 300-308, 1050-1064, and 1068 may be administered topically. In one embodiment, as used herein, “topical administration” refers to application of the compounds of formulas 44-46, 98, 300-308, 1050-1064, and 1068 (and optional carrier) directly to the skin and/or hair.

The topical composition can be in the form of solutions, lotions, salves, creams, ointments, liposomes, sprays, gels, foams, roller sticks, and any other formulation routinely used in dermatology.

Topical administration is used for indications found on the skin, such as hirsutism, alopecia, acne, and excess sebum. The dose will vary, but as a general guideline, the compound will be present in a dermatologically acceptable carrier in an amount of from about 0.01 to 50 w/w %, and more typically from about 0.1 to 10 w/w %. Typically, the dermatological preparation will be applied to the affected area from 1 to 4 times daily. “Dermatologically acceptable” refers to a carrier which may be applied to the skin or hair, and which will allow the drug to diffuse to the site of action. More specifically “site of action”, it refers to a site where inhibition of androgen receptor or degradation of the androgen receptor is desired.

The compounds of formulas I-IX, IA, IB, IC, ID, IIA, IIB, VIIA, VIIB, VIIIA, VIIIB, IXA or IXB, may be used topically to relieve alopecia, especially androgenic alopecia.

The compounds of formulas 44-46, 98, 300-308, 1050-1064, and 1068, may be used topically to relieve alopecia, especially androgenic alopecia.

Androgens have a profound effect on both hair growth and hair loss. In most body sites, such as the beard and pubic skin, androgens stimulate hair growth by prolonging the growth phase of the hair cycle (anagen) and increasing follicle size. Hair growth on the scalp does not require androgens but, paradoxically, androgens are necessary for the balding on the scalp in genetically predisposed individuals (androgenic alopecia) where there is a progressive decline in the duration of anagen and in hair follicle size. Androgenic alopecia is also common in women where it usually presents as a diffuse hair loss rather than showing the patterning seen in men.

While the compounds of formulas I-IX, IA, IB, IC, ID, IIA, IIB, VIIA, VIIB, VIIIA, VIIIB, IXA or IXB will most typically be used to alleviate androgenic alopecia, the compounds may be used to alleviate any type of alopecia.

The compounds of formulas 44-46, 98, 300-308, 1050-1064, and 1068 will most typically be used to alleviate androgenic alopecia, and the compounds may further be used to alleviate any type of alopecia.

Examples of non-androgenic alopecia include, but are not limited to, alopecia areata, alopecia due to radiotherapy or chemotherapy, scarring alopecia, or stress related alopecia.

The compounds of formulas I-IX, IA, IB, IC, ID, IIA, IIB, VIIA, VIIB, VIIIA, VIIIB, IXA or IXB can be applied topically to the scalp and hair to prevent, or treat balding. Further, the compound of formulas I-IX, IA, IB, IC, ID, IIA, IIB, VIIA, VIIB, VIIIA, VIIIB, IXA or IXB can be applied topically in order to induce or promote the growth or regrowth of hair on the scalp.

The compounds of formulas 44-46, 98, 300-308, 1050-1064, and 1068 can be applied topically to the scalp and hair to prevent, or treat balding. Further, the compound of formulas 44-46, 98, 300-308, 1050-1064, and 1068 can be applied topically in order to induce or promote the growth or regrowth of hair on the scalp.

The invention further encompasses topically administering a compound of formula I-IX, IA, IB, IC, ID, IIA, IIB, VIIA, VIIB, VIIIA, VIIIB, IXA or IXB to treat or prevent the growth of hair in areas where such hair growth in not desired.

The invention also encompasses topically administering a compound of formula 44-46, 98, 300-308, 1050-1064, and 1068 to treat or prevent the growth of hair in areas where such hair growth in not desired. One such use will be to alleviate hirsutism. Hirsutism is excessive hair growth in areas that typically do not have hair (e.g., a female face). Such inappropriate hair growth occurs most commonly in women and is frequently seen at menopause. The topical administration of the compounds of formulas 44-46, 98, 300-308, 1050-1064, and 1068 will alleviate this condition leading to a reduction, or elimination of this inappropriate, or undesired, hair growth. The topical administration of the compounds of formulas I-IX, IA, IB, IC, ID, IIA, IIB, VIIA, VIIB, VIIIA, VIIIB, IXA or IXB will also alleviate this condition leading to a reduction, or elimination of this inappropriate, or undesired, hair growth.

The compounds of formulas I-IX, IA, IB, IC, ID, IIA, IIB, VIIA, VIIB, VIIIA, VIIIB, IXA or IXB may be used topically to decrease sebum production. The compounds of formulas 44-46, 98, 300-308, 1050-1064, and 1068 may also be used topically to decrease sebum production. Sebum is composed of triglycerides, wax esters, fatty acids, sterol esters and squalene. Sebum is produced in the acinar cells of the sebaceous glands and accumulates as these cells age. At maturation, the acinar cells lyse, releasing sebum into the luminal duct so that it may be deposited on the surface of the skin.

In some individuals, an excessive quantity of sebum is secreted onto the skin. This can have a number of adverse consequences. It can exacerbate acne, since sebum is the primary food source for Propionbacterium acnes, the causative agent of acne. It can cause the skin to have a greasy appearance, typically considered cosmetically unappealing.

Formation of sebum is regulated by growth factors and a variety of hormones including androgens. The cellular and molecular mechanism by which androgens exert their influence on the sebaceous gland has not been fully elucidated. However, clinical experience documents the impact androgens have on sebum production. Sebum production is significantly increased during puberty, when androgen levels are their highest. The compounds of formulas 44-46, 98, 300-308, 1050-1064, and 1068 inhibit the secretion of sebum and thus reduce the amount of sebum on the surface of the skin. The compounds of formulas 44-46, 98, 300-308, 1050-1064, and 1068 can be used to treat a variety of dermal diseases such as acne or seborrheic dermatitis. Further, the compounds of formulas I-IX, IA, IB, IC, ID, IIA, IIB, VIIA, VIIB, VIIIA, VIIIB, IXA or IXB inhibit the secretion of sebum and thus reduce the amount of sebum on the surface of the skin. The compounds of formulas I-IX, IA, IB, IC, ID, IIA, IIB, VIIA, VIIB, VIIIA, VIIIB, IXA or IXB can be used to treat a variety of dermal diseases such as acne or seborrheic dermatitis.

In addition to treating diseases associated with excess sebum production, the compounds of formulas 44-46, 98, 300-308, 1050-1064, and 1068 can be used to achieve a cosmetic effect. Some consumers believe that they are afflicted with overactive sebaceous glands. They feel that their skin is oily and thus unattractive. These individuals may use the compounds of formulas 44-46, 98, 300-308, 1050-1064, and 1068 to decrease the amount of sebum on their skin. Decreasing the secretion of sebum will alleviate oily skin in individuals afflicted with such conditions.

Further, in addition to treating diseases associated with excess sebum production, the compounds of formulas I-IX, IA, IB, IC, ID, IIA, IIB, VIIA, VIIB, VIIIA, VIIIB, IXA or IXB can also be used to achieve a cosmetic effect. When some consumers believe that they are afflicted with overactive sebaceous glands and they feel that their skin is oily and thus unattractive, these individuals may use the compounds of formulas I-IX, IA, IB, IC, ID, IIA, IIB, VIIA, VIIB, VIIIA, VIIIB, IXA or IXB to decrease the amount of sebum on their skin.

To treat these topical indications, the invention further encompasses cosmetic or pharmaceutical compositions (such as dermatological compositions), comprising at least one of the compounds of formulas 44-46, 98, 300-308, 1050-1064, and 1068. The invention also encompasses cosmetic or pharmaceutical compositions (such as dermatological compositions), comprising at least one of the compounds of formulas I-IX, IA, IB, IC, ID, IIA, IIB, VIIA, VIIB, VIIIA, VIIIB, IXA or IXB. Such dermatological compositions will contain from 0.001% to 10% w/w % of the compound(s) in admixture with a dermatologically acceptable carrier, and more typically, from 0.1 to 5 w/w % of the compounds. Such compositions will typically be applied from 1 to 4 times daily. The reader's attention is directed to Remington's Pharmaceutical Science, Edition 17, Mark Publishing Co., Easton, Pa. for a discussion of how to prepare such formulations.

The compositions of the invention may also include solid preparations such as cleansing soaps or bars. These compositions are prepared according to methods known in the art.

Formulations such as aqueous, alcoholic, or aqueous-alcoholic solutions, or creams, gels, emulsions or mousses, or aerosol compositions with a propellant may be used to treat indications that arise where hair is present. Thus, the composition can also be a hair care composition. Such hair care compositions include, but are not limited to, shampoo, a hair-setting lotion, a treating lotion, a styling cream or gel, a dye composition, or a lotion or gel for preventing hair loss. The amounts of the various constituents in the dermatological compositions are those conventionally used in the fields considered.

Medicinal and cosmetic agents containing the compounds of formulas 44-46, 98, 300-308, 1050-1064, and 1068 will also typically be packaged for retail distribution (i.e., an article of manufacture). Medicinal and cosmetic agents containing the compounds of formulas I-IX, IA, IB, IC, ID, IIA, IIB, VIIA, VIIB, VIIIA, VIIIB, IXA or IXB will also may be packaged for retail distribution (i.e., an article of manufacture). Such articles will be labeled and packaged in a manner to instruct the patient how to use the product. Such instructions will include the condition to be treated, duration of treatment, dosing schedule, etc.

Antiandrogens, such as finasteride or flutamide, have been shown to decrease androgen levels or block androgen action in the skin to some extent but suffer from undesirable systemic effects. An alternative approach is to topically apply a selective androgen receptor degrader (SARD) compound to the affected areas. Such SARD compound would exhibit potent but local inhibition of AR activity, and local degradation of the AR, would not penetrate to the systemic circulation of the subject, or would be rapidly metabolized upon entry into the blood, limiting systemic exposure.

To prepare such pharmaceutical dosage forms, the active ingredient may be mixed with a pharmaceutical carrier according to conventional pharmaceutical compounding techniques. The carrier may take a wide variety of forms depending on the form of preparation desired for administration.

As used herein “pharmaceutically acceptable carriers or diluents” are well known to those skilled in the art. The carrier or diluent may be a solid carrier or diluent for solid formulations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

Oral and Parenteral Administration: In preparing the compositions in oral dosage form, any of the usual pharmaceutical media may be employed. Thus, for liquid oral preparations, such as, suspensions, elixirs, and solutions, suitable carriers and additives include water, glycols, oils, alcohols, flavoring agents, preservatives, coloring agents, and the like. For solid oral preparations such as, powders, capsules, and tablets, suitable carriers and additives include starches, sugars, diluents, granulating agents, lubricants, binders, disintegrating agents, and the like. Due to their ease in administration, tablets and capsules represent the most advantageous oral dosage unit form. If desired, tablets may be sugar coated or enteric coated by standard techniques.

For parenteral formulations, the carrier will usually comprise sterile water, though other ingredients may be included, such as ingredients that aid solubility or for preservation. Injectable solutions may also be prepared in which case appropriate stabilizing agents may be employed.

In some applications, it may be advantageous to utilize the active agent in a “vectorized” form, such as by encapsulation of the active agent in a liposome or other encapsulant medium, or by fixation of the active agent, e.g., by covalent bonding, chelation, or associative coordination, on a suitable biomolecule, such as those selected from proteins, lipoproteins, glycoproteins, and polysaccharides.

Methods of treatment using formulations suitable for oral administration may be presented as discrete units such as capsules, cachets, tablets, or lozenges, each containing a predetermined amount of the active ingredient. Optionally, a suspension in an aqueous liquor or a non-aqueous liquid may be employed, such as a syrup, an elixir, an emulsion, or a draught.

A tablet may be made by compression or molding, or wet granulation, optionally with one or more accessory ingredients. Compressed tablets may be prepared by compressing in a suitable machine, with the active compound being in a free-flowing form such as a powder or granules which optionally is mixed with, for example, a binder, disintegrant, lubricant, inert diluent, surface active agent, or discharging agent. Molded tablets comprised of a mixture of the powdered active compound with a suitable carrier may be made by molding in a suitable machine.

A syrup may be made by adding the active compound to a concentrated aqueous solution of a sugar, for example sucrose, to which may also be added any accessory ingredient(s). Such accessory ingredient(s) may include flavorings, suitable preservative, agents to retard crystallization of the sugar, and agents to increase the solubility of any other ingredient, such as a polyhydroxy alcohol, for example glycerol or sorbitol.

Formulations suitable for parenteral administration may comprise a sterile aqueous preparation of the active compound, which preferably is isotonic with the blood of the recipient (e.g., physiological saline solution). Such formulations may include suspending agents and thickening agents and liposomes or other microparticulate systems which are designed to target the compound to blood components or one or more organs. The formulations may be presented in unit-dose or multi-dose form.

Parenteral administration may comprise any suitable form of systemic delivery. Administration may for example be intravenous, intra-arterial, intrathecal, intramuscular, subcutaneous, intramuscular, intra-abdominal (e.g., intraperitoneal), etc., and may be effected by infusion pumps (external or implantable) or any other suitable means appropriate to the desired administration modality.

Nasal and other mucosal spray formulations (e.g. inhalable forms) can comprise purified aqueous solutions of the active compounds with preservative agents and isotonic agents. Such formulations are preferably adjusted to a pH and isotonic state compatible with the nasal or other mucous membranes. Alternatively, they can be in the form of finely divided solid powders suspended in a gas carrier. Such formulations may be delivered by any suitable means or method, e.g., by nebulizer, atomizer, metered dose inhaler, or the like.

Formulations for rectal administration may be presented as a suppository with a suitable carrier such as cocoa butter, hydrogenated fats, or hydrogenated fatty carboxylic acids.

Transdermal formulations may be prepared by incorporating the active agent in a thixotropic or gelatinous carrier such as a cellulosic medium, e.g., methyl cellulose or hydroxyethyl cellulose, with the resulting formulation then being packed in a transdermal device adapted to be secured in dermal contact with the skin of a wearer.

In addition to the aforementioned ingredients, formulations of this invention may further include one or more ingredient selected from diluents, buffers, flavoring agents, binders, disintegrants, surface active agents, thickeners, lubricants, preservatives (including antioxidants), and the like.

The formulations may be of immediate release, sustained release, delayed-onset release or any other release profile known to one skilled in the art.

For administration to mammals, and particularly humans, it is expected that the physician will determine the actual dosage and duration of treatment, which will be most suitable for an individual and can vary with the age, weight, genetics and/or response of the particular individual.

The methods of the invention comprise administration of a compound at a therapeutically effective amount. The therapeutically effective amount may include various dosages.

In one embodiment, a compound of this invention is administered at a dosage of 1-3000 mg per day. In additional embodiments, a compound of this invention is administered at a dose of 1-10 mg per day, 3-26 mg per day, 3-60 mg per day, 3-16 mg per day, 3-30 mg per day, 10-26 mg per day, 15-60 mg, 50-100 mg per day, 50-200 mg per day, 100-250 mg per day, 125-300 mg per day, 20-50 mg per day, 5-50 mg per day, 200-500 mg per day, 125-500 mg per day, 500-1000 mg per day, 200-1000 mg per day, 1000-2000 mg per day, 1000-3000 mg per day, 125-3000 mg per day, 2000-3000 mg per day, 300-1500 mg per day or 100-1000 mg per day. In one embodiment, a compound of this invention is administered at a dosage of 25 mg per day. In one embodiment, a compound of this invention is administered at a dosage of 40 mg per day. In one embodiment, a compound of this invention is administered at a dosage of 50 mg per day. In one embodiment, a compound of this invention is administered at a dosage of 67.5 mg per day. In one embodiment, a compound of this invention is administered at a dosage of 75 mg per day. In one embodiment, a compound of this invention is administered at a dosage of 80 mg per day. In one embodiment, a compound of this invention is administered at a dosage of 100 mg per day. In one embodiment, a compound of this invention is administered at a dosage of 125 mg per day. In one embodiment, a compound of this invention is administered at a dosage of 250 mg per day. In one embodiment, a compound of this invention is administered at a dosage of 300 mg per day. In one embodiment, a compound of this invention is administered at a dosage of 500 mg per day. In one embodiment, a compound of this invention is administered at a dosage of 600 mg per day. In one embodiment, a compound of this invention is administered at a dosage of 1000 mg per day. In one embodiment, a compound of this invention is administered at a dosage of 1500 mg per day. In one embodiment, a compound of this invention is administered at a dosage of 2000 mg per day. In one embodiment, a compound of this invention is administered at a dosage of 2500 mg per day. In one embodiment, a compound of this invention is administered at a dosage of 3000 mg per day.

The methods may comprise administering a compound at various dosages. For example, the compound may be administered at a dosage of 3 mg, 10 mg, 30 mg, 40 mg, 50 mg, 80 mg, 100 mg, 120 mg, 125 mg, 200 mg, 250 mg, 300 mg, 450 mg, 500 mg, 600 mg, 900 mg, 1000 mg, 1500 mg, 2000 mg, 2500 mg or 3000 mg.

Alternatively, the compound may be administered at a dosage of 0.1 mg/kg/day. The compound may administered at a dosage between 0.2 to 30 mg/kg/day, or 0.2 mg/kg/day, 0.3 mg/kg/day, 1 mg/kg/day, 3 mg/kg/day, 5 mg/kg/day, 10 mg/kg/day, 20 mg/kg/day, 30 mg/kg/day, 50 mg/kg/day or 100 mg/kg/day.

The pharmaceutical composition may be a solid dosage form, a solution, or a transdermal patch. Solid dosage forms include, but are not limited to, tablets and capsules.

The following examples are presented in order to more fully illustrate the preferred embodiments of the invention. They should in no way, however, be construed as limiting the broad scope of the invention.

### EXAMPLES

### Example 1

### Synthesis of SARDs

### Synthesis of Intermediates 9-10

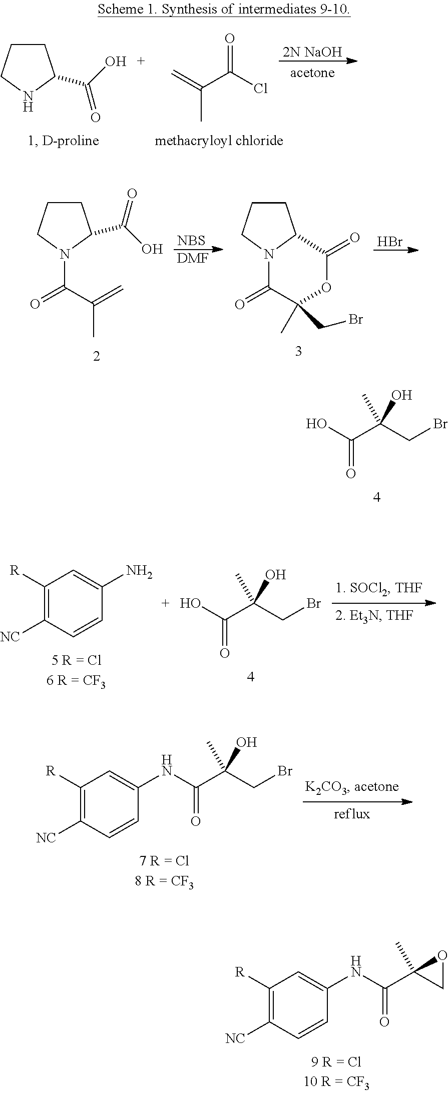

### (2R)-1-Methacryloylpyrrolidin-2-carboxylic Acid (2)

D-Proline (1, 14.93 g, 0.13 mol) was dissolved in 71 mL of 2 N NaOH and cooled in an ice bath. The resulting alkaline solution was diluted with acetone (71 mL). An acetone solution (71 mL) of methacryloyl chloride (13.56 g, 0.13 mol) and 2 N NaOH solution (71 mL) were simultaneously added over 40 min to the aqueous solution of D-proline in an ice bath. The temperature of the mixture was kept at 10-11° C. during the addition of the methacryloyl chloride. After stirring (3 hours (h), room temperature (RT)), the mixture was evaporated in vacuo at a temperature of 35-45° C. to remove acetone. The resulting solution was washed with ethyl ether and was acidified to pH 2 with concentrated HCl. The acidic mixture was saturated with NaCl and was extracted with EtOAc (100 mL×3). The combined extracts were dried over Na₂SO₄, filtered through Celite®, and evaporated in vacuo to give the crude product as a colorless oil. Recrystallization of the oil from ethyl ether and hexanes afforded 16.2 g (68%) of the desired compound as colorless crystals: mp 102.1-103.4° C. (lit. mp 102.5-103.5° C.); the NMR spectrum of this compound demonstrated the existence of two rotamers of the title compound.

¹H NMR (300 MHz, DMSO-d₆) δ 5.28 (s) and 5.15 (s) for the first rotamer, 5.15 (s) and 5.03 (s) for the second rotamer (totally 2H for both rotamers, vinyl CH₂), 4.48-4.44 for the first rotamer, 4.24-4.20 (m) for the second rotamer (totally 1H for both rotamers, CH at the chiral center), 3.57-3.38 (m, 2H, CH₂), 2.27-2.12 (1H, CH), 1.97-1.72 (m, 6H, CH₂, CH, Me); ¹³C NMR (75 MHz, DMSO-d₆) δ for major rotamer 173.3, 169.1, 140.9, 116.4, 58.3, 48.7, 28.9, 24.7, 19.5: for minor rotamer 174.0, 170.0, 141.6, 115.2, 60.3, 45.9, 31.0, 22.3, 19.7; IR (KBr) 3437 (OH), 1737 (C═O), 1647 (CO, COOH), 1584, 1508, 1459, 1369, 1348, 1178 cm⁻¹; [α]_{D}²⁶+80.8 (c=1, MeOH); Anal. Calcd. for C₉H₁₃NO₃: C, 59.00, H, 7.15, N, 7.65. Found: C, 59.13, H, 7.19, N, 7.61.

### (3R,8aR)-3-Bromomethyl-3-methyl-tetrahydro-pyrrolo[2,1-c][1,4]oxazine-1,4-dione (3)

A solution of NBS (23.5 g, 0.132 mol) in 100 mL of DMF was added dropwise to a stirred solution of the (methyl-acryloyl)-pyrrolidine (16.1 g, 88 mmol) in 70 mL of DMF under argon at RT, and the resulting mixture was stirred 3 days. The solvent was removed in vacuo, and a yellow solid was precipitated. The solid was suspended in water, stirred overnight at RT, filtered, and dried to give 18.6 g (81%) (smaller weight when dried 34%) of the titled compound as a yellow solid: mp 158.1-160.3° C.;

¹H NMR (300 MHz, DMSO-d₆) δ 4.69 (dd, J=9.6 Hz, J=6.7 Hz, 1H, CH at the chiral center), 4.02 (d, J=11.4 Hz, 1H, CHHₐ), 3.86 (d, J=11.4 Hz, 1H, CHH_{b}), 3.53-3.24 (m, 4H, CH₂), 2.30-2.20 (m, 1H, CH), 2.04-1.72 (m, 3H, CH₂ and CH), 1.56 (s, 2H, Me); ¹³C NMR (75 MHz, DMSO-d₆) δ 167.3, 163.1, 83.9, 57.2, 45.4, 37.8, 29.0, 22.9, 21.6; IR (KBr) 3474, 1745 (C═O), 1687 (C═O), 1448, 1377, 1360, 1308, 1227, 1159, 1062 cm⁻¹; [α]_{D}²⁶+124.5° (c=1.3, chloroform); Anal. Calcd. for C₉H₁₂BrNO₃: C, 41.24, H, 4.61, N, 5.34. Found: C, 41.46, H, 4.64, N, 5.32.

### (2R)-3-Bromo-2-hydroxy-2-methylpropanoic Acid (4)

A mixture of bromolactone (18.5 g, 71 mmol) in 300 mL of 24% HBr was heated at reflux for 1 h. The resulting solution was diluted with brine (200 mL), and was extracted with ethyl acetate (100 mL×4). The combined extracts were washed with saturated NaHCO₃(100 mL×4). The aqueous solution was acidified with concentrated HCl to pH=1, which, in turn, was extracted with ethyl acetate (100 mL×4). The combined organic solution was dried over Na₂SO₄, filtered through Celite®, and evaporated in vacuo to dryness. Recrystallization from toluene afforded 10.2 g (86%) of the desired compound as colorless crystals: mp 110.3-113.8° C.;

¹H NMR (300 MHz, DMSO-d₆) 3.63 (d, J=10.1 Hz, 1H, CHHₐ), 3.52 (d, J=10.1 Hz, 1H, CHH_{b}), 1.35 (s, 3H, Me); IR (KBr) 3434 (OH), 3300-2500 (COOH), 1730 (C═O), 1449, 1421, 1380, 1292, 1193, 1085 cm; [α]_{D}²⁶+10.5° (c=2.6, MeOH); Anal. Calcd. for C₄H₇BrO₃: C, 26.25, H, 3.86. Found: C, 26.28, H, 3.75.

### (2R)-3-Bromo-N-[4-cyano-3-(trifluoromethyl)phenyl]-2-hydroxy-2-methylpropanamide (8)

Thionyl chloride (46.02 g, 0.39 mol) was added dropwise to a cooled solution (less than 4° C.) of (R)-3-bromo-2-hydroxy-2-methylpropanoic acid (4, 51.13 g, 0.28 mol) in 300 mL of THF under an argon atmosphere. The resulting mixture was stirred for 3 h under the same condition. To this was added Et₃N (39.14 g, 0.39 mol) and stirred for 20 min under the same condition. After 20 min, 5-amino-2-cyanobenzotrifluoride (6, 40.0 g, 0.21 mol), 400 mL of THF were added and then the mixture was allowed to stir overnight at RT. The solvent was removed under reduced pressure to give a solid which was treated with 300 mL of H₂O, and extracted with EtOAc (2×400 mL). The combined organic extracts were washed with saturated NaHCO₃ solution (2×300 mL) and brine (300 mL). The organic layer was dried over MgSO₄ and concentrated under reduced pressure to give a solid which was purified from column chromatography using CH₂Cl₂/EtOAc (80:20) to give a solid. This solid was recrystallized from CH₂Cl₂/hexane to give 55.8 g (73.9%) of (2R)-3-bromo-N-[4-cyano-3-(trifluoromethyl)phenyl]-2-hydroxy-2-methylpropanamide as a light-yellow solid.

¹H NMR (CDCl₃/TMS) δ 1.66 (s, 3H, CH₃), 3.11 (s, 1H, OH), 3.63 (d, J=10.8 Hz, 1H, CH₂), 4.05 (d, J=10.8 Hz, 1H, CH₂), 7.85 (d, J=8.4 Hz, 1H, ArH), 7.99 (dd, J=2.1, 8.4 Hz, 1H, ArH), 8.12 (d, J=2.1 Hz, 1H, ArH), 9.04 (bs, 1H, NH). MS (ESI) 349.0 [M−H]⁻; mp 124-126° C.

### (2R)-3-Bromo-N-(4-cyano-3-chlorophenyl)-2-hydroxy-2-methylpropanamide (7)

Under an argon atmosphere, thionyl chloride (15 mL, 0.20 mol) was added dropwise to a cooled solution (less than 4° C.) of (R)-3-bromo-2-hydroxy-2-methylpropanoic acid (4, 24.3 g, 0.133 mol) in 300 mL of THF at ice-water bath. The resulting mixture stirred for 3 h under the same condition. To this was added Et₃N (35 mL, 0.245 mol) and stirred for 20 min under the same condition. After 20 min, a solution of 4-amino-2-chlorobenzonitrile (5, 15.6 g, 0.10 mol) in 100 mL of THF were added and then the mixture was allowed to stir overnight at RT. The solvent removed under reduced pressure to give a solid, which treated with 300 mL of H₂O, and extracted with EtOAc (2×150 mL). The combined organic extracts washed with saturated NaHCO₃ solution (2×150 mL) and brine (300 mL). The organic layer was dried over MgSO₄ and concentrated under reduced pressure to give a solid, which purified by flash column chromatography using CH₂C₂/EtOAc (80:20) to give a solid. This solid was recrystallized from CH₂Cl₂/hexane to give 31.8 g (73%) of (2R)-3-bromo-N-(4-cyano-3-chlorophenyl)-2-hydroxy-2-methylpropanamide (7) as a light-yellow solid.

¹H NMR (CDCl₃, 400 MHz) δ 1.7 (s, 3H, CH₃), 3.0 (s, 1H, OH), 3.7 (d, 1H, CH), 4.0 (d, 1H, CH), 7.5 (d, 1H, ArH), 7.7 (d, 1H, ArH), 8.0 (s, 1H, ArH), 8.8 (s, 1H, NH). MS: 342 (M+23); mp 129° C.

### (S)-N-(3-Chloro-4-cyanophenyl)-2-methyloxirane-2-carboxamide (9)

A mixture of 3-bromo-N-(4-cyano-3-chlorophenyl)-2-hydroxy-2-methylpropanamide (7, 0.84 mmol) and potassium carbonate (1.68 mmol) in 10 mL acetone was heated to reflux for 30 min. After complete conversion of starting bromide 7 to desired epoxide 9 as monitored by TLC, the solvent was evaporated under reduced pressure to give yellowish residue, which was poured into 10 mL of anhydrous EtOAc. The solution was filtered through Celite® pad to remove K₂CO₃ residue and condensed under reduced pressure to give epoxide 9 as a light yellowish solid.

¹H NMR (CDCl₃, 400 MHz) δ 8.41 (bs, NH), 8.02 (d, J=2.0 Hz, 1H, ArH), 7.91 (dd, J=2.0, 8.4 Hz, 1H, ArH), 7.79 (d, J=2.0 Hz, 1H, ArH), 3.01 (s, 2H), 1.69 (s, 3H). MS (ESI) m/z 235.0 [M−H]⁻.

### Example 2

### Androgen Receptor Binding, Transactivation, Degradation, and Metabolism of Pyrazole, Indole, Benztriazole, and Indazole SARDs

### Ligand Binding Assay

Objective: To determine SARDs binding affinity to the AR-LBD.

Method: hAR-LBD (633-919) was cloned into pGex4t.1. Large scale GST-tagged AR-LBD was prepared and purified using a GST column. Recombinant AR-LBD was combined with [³H]mibolerone (PerkinElmer, Waltham, Mass.) in buffer A (10 mM Tris, pH 7.4, 1.5 mM disodium EDTA, 0.25 M sucrose, 10 mM sodium molybdate, 1 mM PMSF) to determine the equilibrium dissociation constant (K_{d}) of [³H]mibolerone. Protein was incubated with increasing concentrations of [³H]mibolerone with and without a high concentration of unlabeled mibolerone at 4° C. for 18 h in order to determine total and non-specific binding. Non-specific binding was then subtracted from total binding to determine specific binding and non-linear regression for ligand binding curve with one site saturation to determine the K_{d} of mibolerone.

Increasing concentrations of SARDs or DHT (range: 10⁻¹² to 10⁻² M) were incubated with [³H]mibolerone and AR LBD using the conditions described above. Following incubation, the ligand bound AR-LBD complex was isolated using Bio Gel HT® hydroxyapatite, washed and counted in a scintillation counter after adding scintillation cocktail. Values are expressed as Kᵢ and reported in Table 1.

Transactivation Assay with wt AR

Objective: To determine the effect of SARDs on androgen-induced transactivation of AR wildtype (wt).

Method: HEK-293 cells were plated at 125,000 cells/well of a 24 well plate in DME+5% csFBS without phenol red. Cells were transfected with 0.25 ug GRE-LUC, 10 ng CMV-renilla LUC, and 50 ng CMV-hAR(wt) using Lipofectamine transfection reagent in optiMEM medium. Medium was changed 24 h after transfection to DME+5% csFBS without phenol red and treated with a dose response of various drugs (1 μM to 10 μM). SARDs and antagonists were treated in combination with 0.1 nM R1881. Luciferase assay was performed 24 h after treatment on a Biotek synergy 4 plate reader. Firefly luciferase values were normalized to renilla luciferase values. Results are reported in Table 1 as IC₅₀ values.

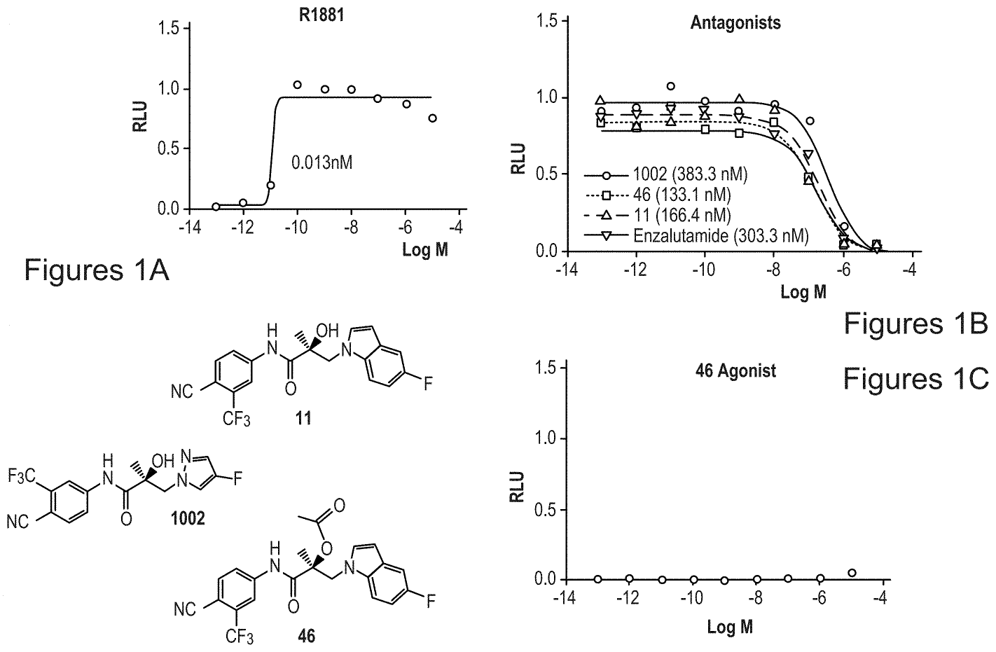

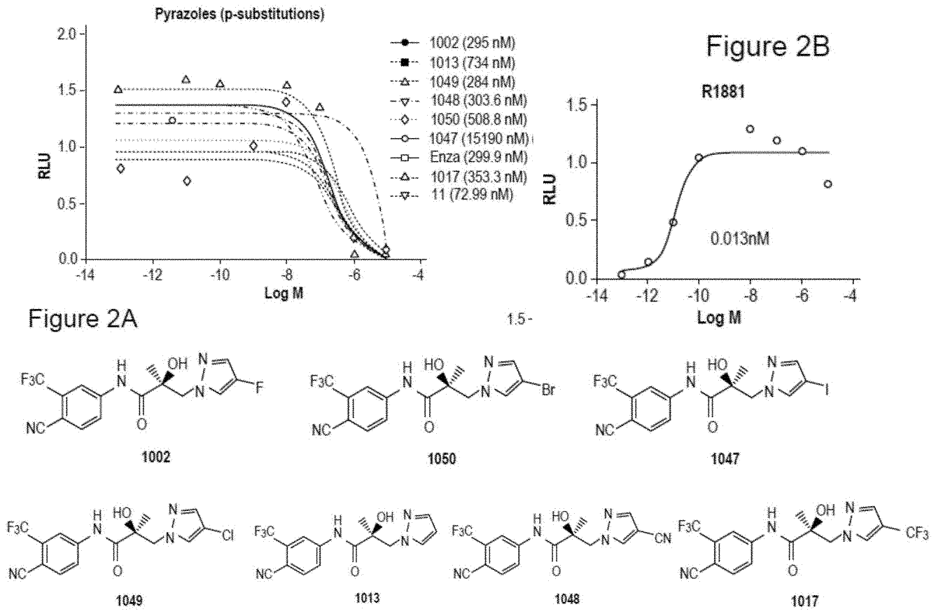

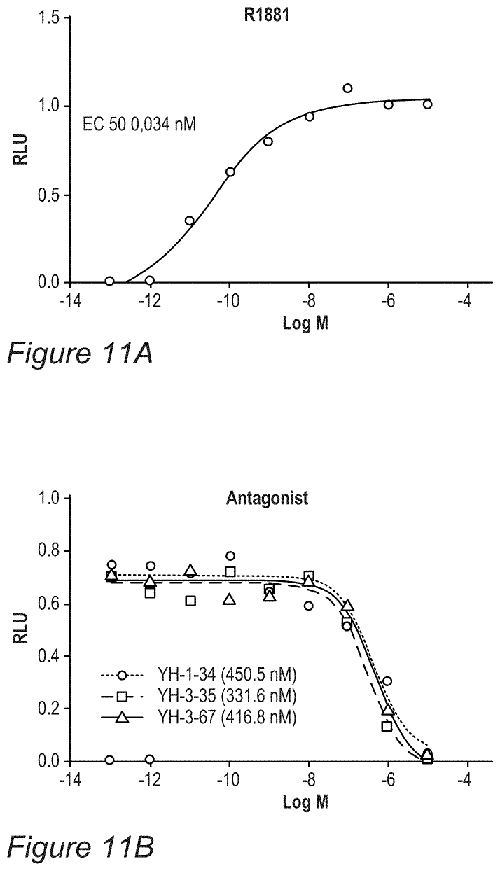

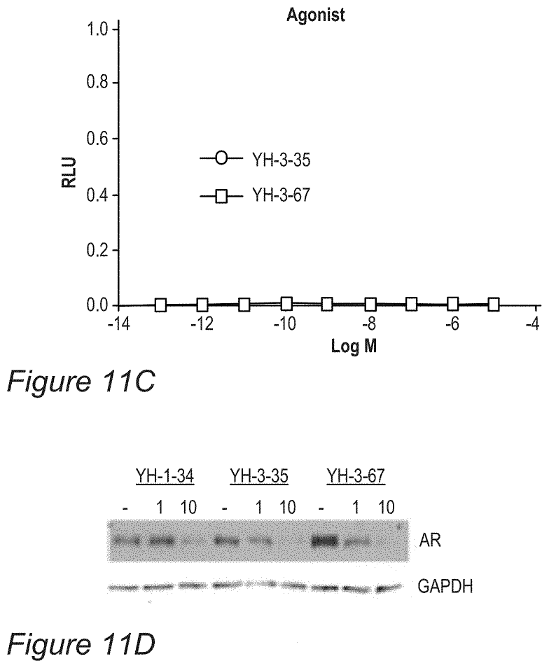

Representative examples of in vitro transactivation antagonism (in the presence of R1881) and agonism (in the absence of R1881) experiments are shown in FIGS. 1A-1C (11, 1002, 46 structures shown in the figure and in Table 1), FIGS. 2A-2B (1002, 1050, 1047, 1013, 1049, 1048, and 1017 structures are shown in the figure and in Table 1) and FIG. 11A-11C (1068, 1061, and 1002 structures are shown in Table 1). These experiments demonstrated that the SARDs of this invention, in addition to degrading the AR and AR-SV, also reversibly inhibited R1881 induced agonist activity of the wtAR but possess no intrinsic agonist activity.

### Plasmid Constructs and Transient Transfection

Human AR cloned into CMV vector backbone was used for the transactivation study. HEK-293 cells were plated at 120,000 cells per well of a 24 well plate in DME+5% csFBS. The cells were transfected using Lipofectamine (Invitrogen, Carlsbad, Calif.) with 0.25 g GRE-LUC, 0.01 μg CMV-LUC (renilla luciferase) and 25 ng of the AR. The cells were treated 24 hrs after transfection as indicated in the figures and the luciferase assay performed 48 hrs after transfection. Data are represented as IC₅₀ obtained from four parameter logistics curve.

### Lncap Gene Expression Assay

Method: LNCaP cells were plated at 15,000 cells/well of a 96 well plate in RPMI+1% csFBS without phenol red. Forty-eight hours after plating, cells were treated with a dose response of SARDs. Twenty four hours after treatment, RNA was isolated using cells-to-ct reagent, cDNA synthesized, and expression of various genes was measured by realtime rtPCR (ABI 7900) using taqman primers and probes. Gene expression results were normalized to GAPDH. This method was adapted to LNCaP-ARV7 cells in Example 12.

### LNCaP Growth Assay

Method: LNCaP cells were plated at 10,000 cells/well of a 96 well plate in RPMI+1% csFBS without phenol red. Cells were treated with a dose response of SARDs. Three days after treatment, cells were treated again. Six days after treatment, cells were fixed and cell viability was measured by SRB assay. This method was adapted to LNCaP-ARV7 cells in Example 12.

### LNCaP or AD1 Degradation

Method: LNCaP or AD1 cells expressing full length AR were plated at 750,000-1,000,000 cells/well of a 6 well plate in growth medium (RPMI+10% FBS). Twenty four hours after plating, medium was changed to RPMI+1% csFBS without phenol red and maintained in this medium for 2 days. Medium was again changed to RPMI+1% csFBS without phenol red and cells were treated with SARDs (1 nM to 10 μM) in combination with 0.1 nM R1881. After 24 h of treatment, cells were washed with cold PBS and harvested. Protein was extracted using salt-containing lysis buffer with three freeze-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma. Results are reported in Table 1 (Full Lenghth AR or AR-FL). This method was adapted to LNCaP-ARV7 cells in Example 12.

### 22RV1 and D567es Degradation

Method: 22RV1 and D567es cells expressing AR splice variants were plated at 750,000-1,000,000 cells/well of a 6 well plate in growth medium (RPMI+10% FBS). Twenty four hours after plating, medium was changed and treated. After 24-30 h of treatment, cells were washed with cold PBS and harvested. Protein was extracted using salt-containing lysis buffer with three freeze-thaw cycles. Protein concentration was estimated and five microgram of total protein was loaded on a SDS-PAGE, fractionated, and transferred to a PVDF membrane. The membrane was probed with AR N-20 antibody from SantaCruz and actin antibody from Sigma. Results are reported in Table 1 (S.V. AR or AR-SV).

### 22RV1 Growth And Gene Expression

Methods: Cell growth was evaluated as described before by SRB assay. Cells were plated in a 96 well plate in full serum and treated for 6 days with medium change after day 3. Gene expression studies were performed in 22RV1 cells plated in 96 well plate at 10,000 cells/well in RPMI+10% FBS. Twenty four hours after plating, cells were treated for 3 days and gene expression studies were performed as described before.

Determination of Metabolic Stability (in vitro CLᵢₙₜ) of Test Compounds

### Phase I Metabolism

The assay was done in a final volume of 0.5 mL in duplicates (n=2). Test compound (1 M) was pre-incubated for 10 minutes at 37° C. in 100 mM Tris-HCl, pH 7.5 containing 0.5 mg/mL liver microsomal protein. After pre-incubation, reaction was started by addition of 1 mM NADPH (pre-incubated at 37° C.). Incubations were carried out in triplicate and at various time-points (0, 5, 10, 15, 30 and 60 minutes) 100 L aliquots were removed and quenched with 100 L of acetonitrile containing internal standard. Samples were vortex mixed and centrifuged at 4000 rpm for 10 minutes. The supernatants were transferred to 96 well plates and submitted for LC-MS/MS analysis. As control, sample incubations done in absence of NADPH were included. From % PCR (% Parent Compound Remaining), rate of compound disappearance is determined (slope) and in vitro CLᵢₙₜ(μL/min/mg protein) was calculated.

### Metabolic Stability in Phase I & Phase II Pathways

In this assay, test compound was incubated with liver microsomes and disappearance of drug was determined using discovery grade LC-MS/MS. To stimulate Phase II metabolic pathway (glucuronidation), UDPGA and alamethicin was included in the assay.

### LC-MS/MS Analysis:

The analysis of the compounds under investigation was performed using LC-MS/MS system consisting of Agilent 1100 HPLC with an MDS/Sciex 4000 Q-Trap™ mass spectrometer. The separation was achieved using a C₁₈ analytical column (Alltima™, 2.1×100 mm, 3 μm) protected by a C₁₈ guard cartridge system (SecurityGuard™ ULTRA Cartridges UHPLC for 4.6 mm ID columns, Phenomenex). Mobile phase was consisting of channel A (95% acetonitrile+5% water+0.1% formic acid) and channel C (95% water+5% acetonitrile+0.1% formic acid) and was delivered at a flow rate of 0.4 mL/min. The volume ratio of acetonitrile and water was optimized for each of the analytes. Multiple reaction monitoring (MRM) scans were made with curtain gas, collision gas, nebulizer gas, and auxiliary gas optimized for each compound, and source temperature at 550° C. Molecular ions were formed using an ion spray voltage of −4200 V (negative mode). Declustering potential, entrance potential, collision energy, product ion mass, and cell exit potential were optimized for each compound. Results are reported in Table 1.

### Log P: Octanol-Water Partition Coefficient (Log P)

Log P is the log of the octanol-water partition coefficient, commonly used early in drug discovery efforts as a rough estimate of whether a particular molecule is likely to cross biological membranes. Log P was calculated using ChemDraw Ultra version is 12.0.2.1016 (Perkin-Elmer, Waltham, Mass. 02451). Calculated Log P values are reported in Table 1 in the column labeled ‘Log P (−0.4 to +5.6)’. Lipinski's rule of five is a set of criteria intended to predict oral bioavailability. One of these criteria for oral bioavailability is that the Log P is between the values shown in the column heading (−0.4 (relatively hydrophilic) to +5.6 (relatively lipophilic) range), or more generally stated <5. One of the goals of SARD design was to improve water solubility. The monocyclic templates of this invention such as the pyrazoles, indazoles, tetrazoles, etc. were more water soluble than earlier analogs. For instance, one may compare the Log P values of SARDs from other templates, e.g., alkyl-amine 17, indoline 100 and indole 11, to the monocyclics of the invention (44-46, 98, 300-308, 1050-1064, and 1068). Results are reported in Table 1.

TABLE 1
In vitro screening of LBD binding (Kᵢ), AR antagonism (IC₅₀), SARD activity, and metabolic stability

| | | | | wtAR Binding | | SARD Activity | | |
|---|---|---|---|---|---|---|---|---|
| | | | | (Kᵢ (left)) & | | (% inh): Full | | DMPK |
| | | | | Transactivation | | Length (left) and | | (MLM) |
| | | | | (IC₅₀ (right)) | | S.V. (right) | | T_{1/2} |
| | | | | (nM) | | Full Length | | (min) & |
| | | Log P | | Kᵢ (nM) | | % inhi- | S.V. % | CLᵢₙₜ |
| Compound | | (−0.4 to | | (DHT = | IC₅₀ | bition at | inhibition | (μL/ |
| # | Structure | +5.6) | M.W. | 1 nM | (nM) | 1, 10 μM | at 10 μM | min/mg) |
| Enobosarm (agonist) | 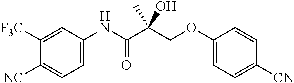 [SMILES: C[C@](O)(COc1ccc(C#N)cc1)C(=O)Nc1ccc(C#N)c(C(F)(F)F)c1] | 3.44 | 389.89 | 20.21 | ~20 (EC₅₀) | Not applicable | Not applicable | |
| R- Bicalut- amide | 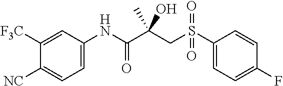 [SMILES: C[C@](O)(CS(=O)(=O)c1ccc(F)cc1)C(=O)Nc1ccc(C#N)c(C(F)(F)F)c1] | 2.57 | 430.37 | 508.84 | 248.2 | 0 | 0 | |
| Enzalut- amide | 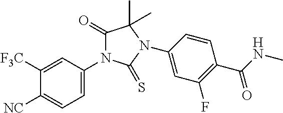 [SMILES: CNC(=O)c1ccc(N2C(=S)N(c3ccc(C#N)c(C(F)(F)F)c3)C(=O)C2(C)C)cc1F] | 4.56 | 464.44 | 3641.29 | 216.3 | 0 | 0 | |
| ARN-509 (Apalut- amide) | 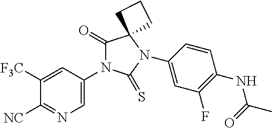 | 3.47 | 477.43 | 1452.29 | | 0 | 0 | |
| 17 | 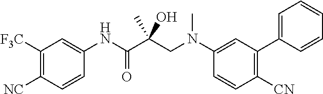 [SMILES: CN(C[C@](C)(O)C(=O)Nc1ccc(C#N)c(C(F)(F)F)c1)c1ccc(C#N)c(-c2ccccc2)c1] | 5.69 | 478.48 | 28.4 | 95 | 80 | 80 | |
| 100 | 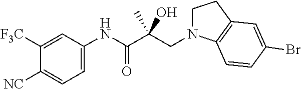 [SMILES: C[C@](O)(CN1CCc2cc(Br)ccc21)C(=O)Nc1ccc(C#N)c(C(F)(F)F)c1] | 4.62 | 468.27 | 197.67 | 530.95 | 60 | 41 | 66.87 10.38 |
| 11 | 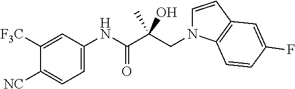 [SMILES: C[C@](O)(Cn1ccc2cc(F)ccc21)C(=O)Nc1ccc(C#N)c(C(F)(F)F)c1] | 3.47 | 405.35 | 267.39 | 85.10 | 65-83 | 60-100 | 12.35 56.14 |
| 44 | 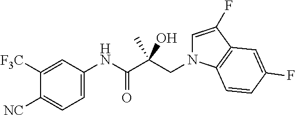 [SMILES: C[C@](O)(Cn1cc(F)c2cc(F)ccc21)C(=O)Nc1ccc(C#N)c(C(F)(F)F)c1] | 3.63 | | 317.64 | 274.3 | 72 | 84 | |
| 45 | 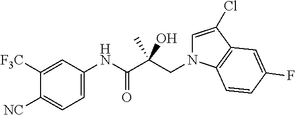 [SMILES: C[C@](O)(Cn1cc(Cl)c2cc(F)ccc21)C(=O)Nc1ccc(C#N)c(C(F)(F)F)c1] | 4.03 | | 754.7 | 366.9 | 60 | 80 | |
| 46 | 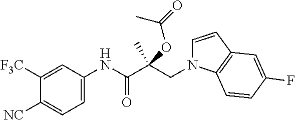 [SMILES: CC(=O)O[C@@](C)(Cn1ccc2cc(F)ccc21)C(=O)Nc1ccc(C#N)c(C(F)(F)F)c1] | 3.7 | | 134.19 | 133.1 | 90 | 100 | |
| 98 | 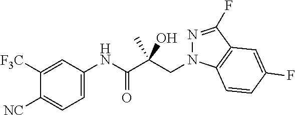 [SMILES: C[C@](O)(Cn1nc(F)c2cc(F)ccc21)C(=O)Nc1ccc(C#N)c(C(F)(F)F)c1] | 3.71 | 424.31 | 605.4 | 101.5 | 37, 81 | 53 | |
| 300 | 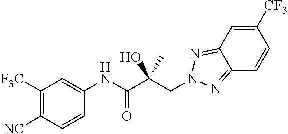 [SMILES: C[C@](O)(Cn1nc2ccc(C(F)(F)F)cc2n1)C(=O)Nc1ccc(C#N)c(C(F)(F)F)c1] | 4.25 | | | No effect | | | |
| 301 | 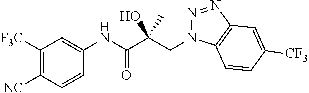 [SMILES: C[C@](O)(Cn1nnc2cc(C(F)(F)F)ccc21)C(=O)Nc1ccc(C#N)c(C(F)(F)F)c1] | 3.87 | | | — | | | |
| 302 | 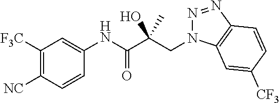 [SMILES: C[C@](O)(Cn1nnc2ccc(C(F)(F)F)cc21)C(=O)Nc1ccc(C#N)c(C(F)(F)F)c1] | 3.87 | | | — | | | |
| 301/302 | 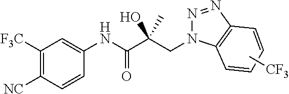 | 3.87 | | | No effect | | | |
| 303 | 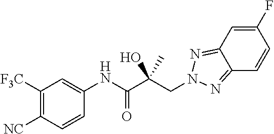 [SMILES: C[C@](O)(Cn1nc2ccc(F)cc2n1)C(=O)Nc1ccc(C#N)c(C(F)(F)F)c1] | 3.48 | | 3615 | 277 | 70 | 0 | |
| 304 | 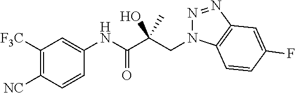 [SMILES: C[C@](O)(Cn1nnc2cc(F)ccc21)C(=O)Nc1ccc(C#N)c(C(F)(F)F)c1] | 3.11 | | | 687 | 60 | 0 | |
| 305 | 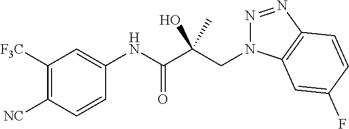 [SMILES: C[C@](O)(Cn1nnc2ccc(F)cc21)C(=O)Nc1ccc(C#N)c(C(F)(F)F)c1] | 3.11 | | 1476 | 560 | 40 | 0 | |
| 307 | 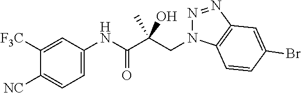 [SMILES: C[C@](O)(Cn1nnc2cc(Br)ccc21)C(=O)Nc1ccc(C#N)c(C(F)(F)F)c1] | 3.78 | | | 2594 nM | | | |
| 308 | 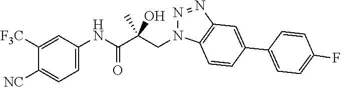 [SMILES: C[C@](O)(Cn1nnc2cc(-c3ccc(F)cc3)ccc21)C(=O)Nc1ccc(C#N)c(C(F)(F)F)c1] | 4.79 | | | No effect | | | |
| 1002 | 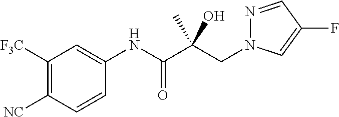 [SMILES: C[C@](O)(Cn1cc(F)cn1)C(=O)Nc1ccc(C#N)c(C(F)(F)F)c1] | 2.03 | 356.27 | No binding | 199.36 | 100 | 100 | 77.96 0.89 |
| 1002 tartarate (1002 Tart.) | 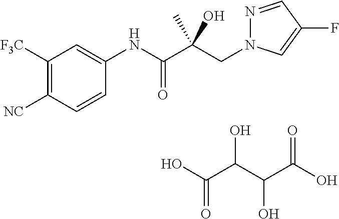 [SMILES: C[C@](O)(Cn1cc(F)cn1)C(=O)Nc1ccc(C#N)c(C(F)(F)F)c1 ; O=C(O)C(O)C(O)C(=O)O] | | 506.36 | | 125.2 | | | |
| 1013 | 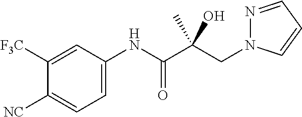 [SMILES: C[C@](O)(Cn1cccn1)C(=O)Nc1ccc(C#N)c(C(F)(F)F)c1] | 1.87 | 338.28 | 7398 | 1441.58 | 0 | | |
| 1017 | 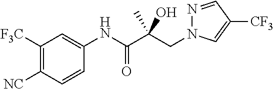 [SMILES: C[C@](O)(Cn1cc(C(F)(F)F)cn1)C(=O)Nc1ccc(C#N)c(C(F)(F)F)c1] | 2.79 | 406.28 | 898.23 | 71.2 | 80 | 100 | Infinity 0 |
| 1022 | 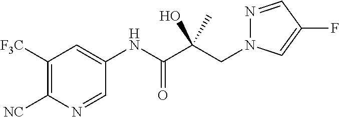 [SMILES: C[C@](O)(Cn1cc(F)cn1)C(=O)Nc1cnc(C#N)c(C(F)(F)F)c1] | 1.11 | 357.26 | No binding | 62.2 | 54 | 81 | |
| 1045 | 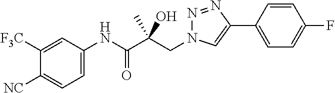 [SMILES: C[C@](O)(Cn1cc(-c2ccc(F)cc2)nn1)C(=O)Nc1ccc(C#N)c(C(F)(F)F)c1] | 3.73 | 433.36 | No binding | 383.3 | 84 | | |
| 1047 | 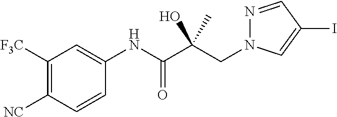 [SMILES: C[C@](O)(Cn1cc(I)cn1)C(=O)Nc1ccc(C#N)c(C(F)(F)F)c1] | 3.23 | 464.18 | | 2038 | | | |
| 1048 | 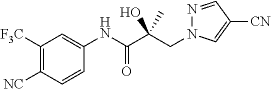 [SMILES: C[C@](O)(Cn1cc(C#N)cn1)C(=O)Nc1ccc(C#N)c(C(F)(F)F)c1] | 1.90 | 363.29 | 1499 | 44.5 | 90 | 100 | |
| 1049 | 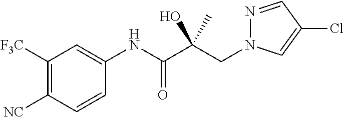 [SMILES: C[C@](O)(Cn1cc(Cl)cn1)C(=O)Nc1ccc(C#N)c(C(F)(F)F)c1] | 2.43 | 372.73 | >10000 | 135.7 | 71 | 34 | |
| 1050 | 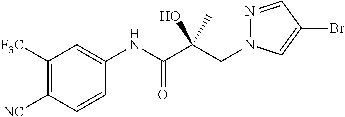 [SMILES: C[C@](O)(Cn1cc(Br)cn1)C(=O)Nc1ccc(C#N)c(C(F)(F)F)c1] | 2.70 | 417.18 | >10000 | 427 | 42 | 0 | |
| 1051 | 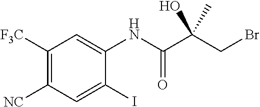 [SMILES: C[C@](O)(CBr)C(=O)Nc1cc(C(F)(F)F)c(C#N)cc1I] | 3.93 | 477.02 | | No effect | | | |
| 1052 | 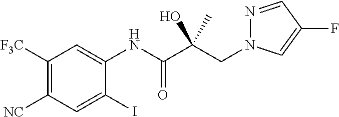 [SMILES: C[C@](O)(Cn1cc(F)cn1)C(=O)Nc1cc(C(F)(F)F)c(C#N)cc1I] | 3.38 | 482.17 | | 5450 | | | |
| 1053 | 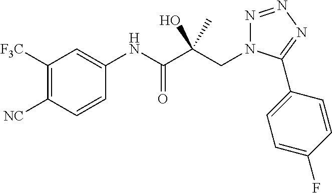 [SMILES: C[C@](O)(Cn1nnnc1-c1ccc(F)cc1)C(=O)Nc1ccc(C#N)c(C(F)(F)F)c1] | 3.44 | 434.35 | | No effect | | | |
| 1054 | 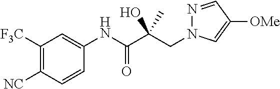 [SMILES: COc1cnn(C[C@](C)(O)C(=O)Nc2ccc(C#N)c(C(F)(F)F)c2)c1] | 1.74 | 368.31 | | — | 0 | 0 | |
| 1055 | 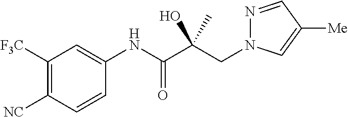 [SMILES: Cc1cnn(C[C@](C)(O)C(=O)Nc2ccc(C#N)c(C(F)(F)F)c2)c1] | 2.36 | 352.31 | 1552 | 8087 | | | |
| 1057 (Racemate) | 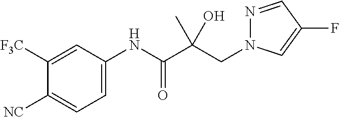 [SMILES: CC(O)(Cn1cc(F)cn1)C(=O)Nc1ccc(C#N)c(C(F)(F)F)c1] | 2.03 | 356.27 | | 312.5 | 1, 15 | | |
| 1058 | 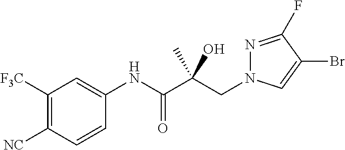 [SMILES: C[C@](O)(Cn1cc(Br)c(F)n1)C(=O)Nc1ccc(C#N)c(C(F)(F)F)c1] | 3.32 | 435.17 | 606.5 | 83.7 | 70 | 80 | |
| 1059 | 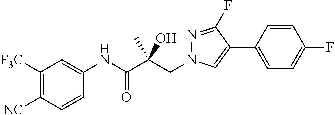 [SMILES: C[C@](O)(Cn1cc(-c2ccc(F)cc2)c(F)n1)C(=O)Nc1ccc(C#N)c(C(F)(F)F)c1] | 4.33 | 450.36 | 600.58 | 285.1 | | toxic | |
| 1060 | 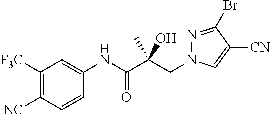 [SMILES: C[C@](O)(Cn1cc(C#N)c(Br)n1)C(=O)Nc1ccc(C#N)c(C(F)(F)F)c1] | 3.14 | 442.19 | 202.3 | 180.5 | 41, 23 | 32 | |
| 1061 | 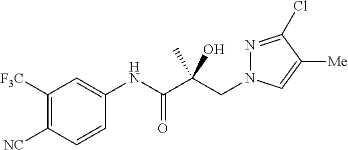 [SMILES: Cc1cn(C[C@](C)(O)C(=O)Nc2ccc(C#N)c(C(F)(F)F)c2)nc1Cl] | 3.26 | 386.76 | 1345.6 | 331.6 | 41, 83 | | |
| 1062 | 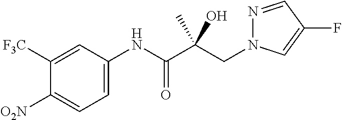 [SMILES: C[C@](O)(Cn1cc(F)cn1)C(=O)Nc1ccc([N+](=O)[O-])c(C(F)(F)F)c1] | 2.03 | 376.24 | | Partial | | | |
| 1062a | 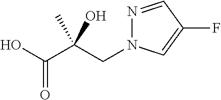 | −0.18 | 188.16 | | No effect | | | |
| 1063 | 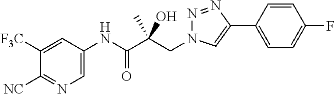 [SMILES: C[C@](O)(Cn1cc(-c2ccc(F)cc2)nn1)C(=O)Nc1cnc(C#N)c(C(F)(F)F)c1] | 2.82 | 434.35 | 1486 | 216.9 | | | |
| 1065 | 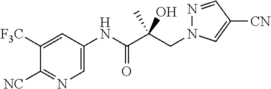 [SMILES: C[C@](O)(Cn1cc(C#N)cn1)C(=O)Nc1cnc(C#N)c(C(F)(F)F)c1] | 3.14 | 451.63 | 89.34 | 59.4 | 30 | 28 | |
| 1066 | 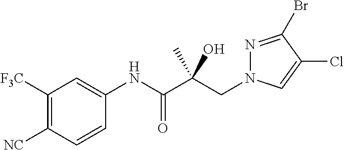 [SMILES: C[C@](O)(Cn1cc(Cl)c(Br)n1)C(=O)Nc1ccc(C#N)c(C(F)(F)F)c1] | 3.66 | 451.63 | 4934.871 | 138.2 | | | |
| 1067 | 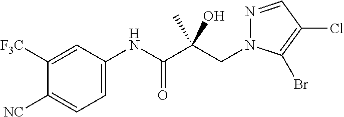 [SMILES: C[C@](O)(Cn1ncc(Cl)c1Br)C(=O)Nc1ccc(C#N)c(C(F)(F)F)c1] | 3.14 | 451.63 | 558.7 | 84 (1002 in the same exp: 128 nM) | 62, 88 | 73 | |
| 1068 | 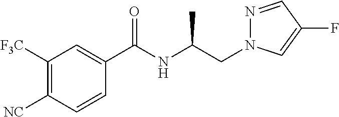 [SMILES: C[C@@H](Cn1cc(F)cn1)NC(=O)c1ccc(C#N)c(C(F)(F)F)c1] | 2.73 | 340.28 | no binding | 416.8 | 62, 96 | | |

### Example 3

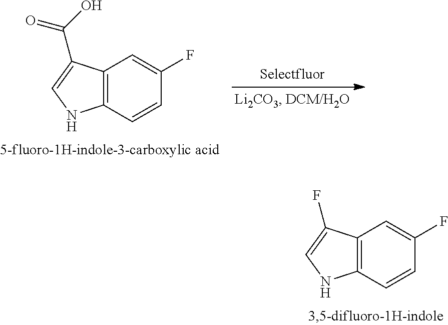 [SMILES: O=C(O)c1c[nH]c2ccc(F)cc12 ; Fc1ccc2[nH]cc(F)c2c1]

### Synthesis of 3,5-difluoro-1H-indole

To a 50 mL round-bottle flask with a magnetic stirring bar were added Selectfluor® (872 mg, 2.0 mmol, 2.0 equiv), Li₂CO₃ (296 mg, 4.0 mmol, 4.0 equiv), dichloromethane (3.3 mL) and water (1.7 mL). Then carboxylic acid (1.0 mmol, 1.0 equiv) was added. The reaction mixture was stirred for 2 hours in ice bath. The reaction mixture was diluted with water (40 mL), followed by extracting with DCM (20 mL×2). The combined organic extracts were washed with brine, dried over anhydrous sodium sulfate and concentrated in vacuo. The crude product was purified by flash column chromatography (n-hexane:DCM=2:1) to afford 3,5-difluoro-1H-indole as deep brown oil. Yield=68%;

MS (ESI) m/z 154.83[M+H]⁺; 152.03 [M−H]⁻;

¹H NMR (CDCl₃, 400 MHz) δ 7.86 (bs, 1H, NH), 7.25 (dd, J=9.2, 2.4 Hz, 1H), 7.20-7.16 (m, 1H), 6.97 (t, J=2.6 Hz, 1H), 6.93 (dd, J=9.2, 2.4 Hz, 1H);

¹⁹F NMR (CDCl₃) δ −123.99 (d, J_{F-F}=2.8 Hz), −174.74 (d, J_{F-F}=4.0 Hz).

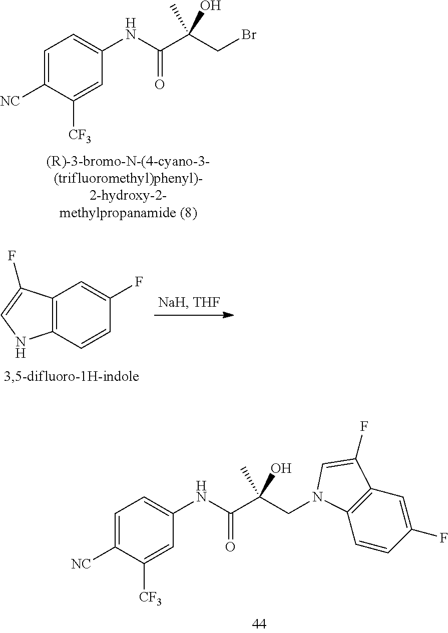 [SMILES: C[C@](O)(CBr)C(=O)Nc1ccc(C#N)c(C(F)(F)F)c1 ; Fc1ccc2[nH]cc(F)c2c1 ; C[C@](O)(Cn1cc(F)c2cc(F)ccc21)C(=O)Nc1ccc(C#N)c(C(F)(F)F)c1]

### (S)-N-(4-Cyano-3-(trifluoromethyl)phenyl)-3-(3,5-difluoro-1H-indol-1-yl)-2-hydroxy-2-methylpropanamide (44)

To a dry, nitrogen-purged 50 mL round-bottom flask equipped with a dropping funnel under argon atmosphere, NaH of 60% dispersion in mineral oil (63 mg, 1.56 mmol) was added in 10 mL of anhydrous THF solvent in the flask at ice-water bath, and 3,5-difluoro-1H-indole (120 mg, 0.78 mmol) was stirred 30 min at the ice-water bath. Into the flask, (R)-3-bromo-N-(4-cyano-3-(trifluoromethyl)phenyl)-2-hydroxy-2-methylpropanamide (8) (275 mg, 0.78 mmol) in 10 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=1/2 to produce 44 as white solid as white powder.

Yield 53%;

MS (ESI) m/z 424.11[M+H]⁺; 423.11 [M−H]⁻;

HRMS (ESI) m/z calcd for C₂₀H₁₅F₅N₃O₂[M+H]⁺; Exact Mass: 424.1084 [M+H]⁺. Found: 424.1065 [M+H]⁺.

HPLC: t_{R} 2.77 min, purity 99.06%, UV (λ_{abs}) 196.45, 270.45 nm

¹H NMR (CDCl₃, 400 MHz) δ 8.80 (bs, 1H, NH), 7.89 (d, J=1.6 Hz, 1H), 7.77 (dd, J=8.4, 1.6 Hz, 1H), 7.74 (d, J=8.4 Hz, 1H), 7.33-7.29 (m, 1H), 7.20 (dd, J=9.0, 2.4 Hz, 1H), 6.99 (t, J=2.8 Hz, 1H), 6.97 (td, J=9.0, 2.4 Hz, 1H), 4.56 (d, J=14.8 Hz, 1H), 4.24 (d, J=14.8 Hz, 1H), 2.57 (s, OH), 1.61 (s, 3H);

¹³C NMR (CDCl₃, 100 MHz) δ 172.3, 157.5 (d, J_{F-F}=235 Hz), 140.9, 135.8, 134.1 (d, J_{F-F}=32.8 Hz), 130.4 (d, J_{F-F}=4.5 Hz), 123.4, 121.9, 120.6, 117.4 (q, J_{F-F}=4.9 Hz), 115.3, 113.1 (d, J_{F-F}=2.59 Hz), 111.1 (d, J_{F-F}=9.3 Hz), 105.0, 102.3, 102.2, 102.0 (d, J_{F-F}=25 Hz), 77.6, 53.9, 24.2;

¹⁹F NMR (CDCl₃) δ −62.25, −123.48 (d, J_{F-F}=3.2 Hz), −173.54 (d, J_{F-F}=2.8 Hz); assigned by 2D NMR as NOE and COSY.

### (S)-3-(3-Chloro-5-fluoro-1H-indol-1-yl)-N-(4-cyano-3-(trifluoromethyl)phenyl)-2-hydroxy-2-methylpropanamide (45)

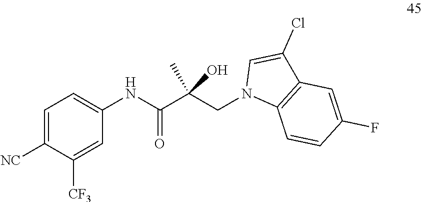 [SMILES: C[C@](O)(Cn1cc(Cl)c2cc(F)ccc21)C(=O)Nc1ccc(C#N)c(C(F)(F)F)c1]

To a dry, nitrogen-purged 50 mL round-bottom flask equipped with a dropping funnel under argon atmosphere, NaH of 60% dispersion in mineral oil (167 mg, 2.5 mmol) was added in 10 mL of anhydrous THF solvent in the flask at ice-water bath, and 3-chloro-5-fluoro-1H-indole (170 mg, 1 mmol) was stirred 30 min at the ice-water bath. Into the flask, (R)-3-bromo-N-(4-cyano-3-(trifluoromethyl)phenyl)-2-hydroxy-2-methylpropanamide (8) (351 mg, 1 mmol) in 10 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=1/2 to produce 45 as white solid as white powder.

Yield 58%;

MS (ESI) m/z 440.08 [M+H]⁺; 439.01 [M−H]−;

HRMS (ESI) m/z calcd for C₂H₁₅ClF₄N₃O₂ Exact Mass: m/z C₂H₁₅ClF₄N₃O₂: 440.0789 [M+H]⁺; 440.0797 [M+H]⁺;

HPLC: tR 2.89 min, purity 99.06%;

UV (λ_{abs}) 196.45, 270.45 nm;

¹H NMR (CDCl₃, 400 MHz) δ 8.76 (bs, 1H, NH), 7.86 (s, 1H), 7.78-7.73 (m, 2H), 7.34 (dd, J=9.2, 4.0 Hz, 1H), 7.29 (dd, J=8.8, 2.4 Hz, 1H), 7.17 (s, 1H), 6.97 (td, J=9.2, 2.4 Hz, 1H), 4.58 (d, J=14.8 Hz, 1H), 4.28 (d, J=14.8 Hz, 1H), 2.64 (s, OH), 1.61 (s, 3H);

¹³C NMR (CDCl₃, 100 MHz) δ 172.3, 157.5 (d, J_{F-F}=235 Hz), 140.8, 135.8, 134.0 (d, J_{F-F}=32 Hz), 132.6, 126.9, 126.2 (d, J_{F-F}=10 Hz), 123.4, 117.4 (q, J_{F-F}=4.9 Hz), 115.3, 112.0 (d, J_{F-F}=26.4 Hz), 111.1 (d, J_{F-F}=9.5 Hz), 106.1, 106.0, 105.0, 103.5 (d, J_{F-F}=25 Hz), 77.5, 53.8, 24.2.

¹⁹F NMR (CDCl₃) δ −62.25, −12.76; assigned by 2D NMR as NOE and COSY.

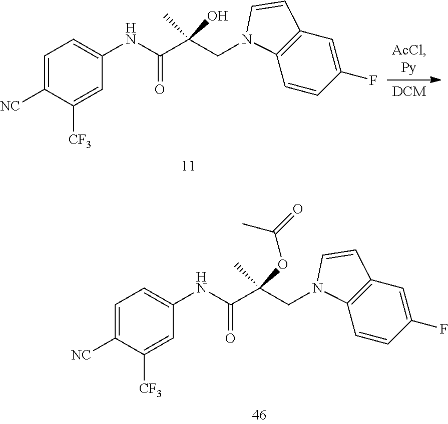 [SMILES: C[C@](O)(Cn1ccc2cc(F)ccc21)C(=O)Nc1ccc(C#N)c(C(F)(F)F)c1 ; CC(=O)O[C@@](C)(Cn1ccc2cc(F)ccc21)C(=O)Nc1ccc(C#N)c(C(F)(F)F)c1]

### (S)-1-((4-Cyano-3-(trifluoromethyl)phenyl)amino)-3-(5-fluoro-1H-indol-1-yl)-2-methyl-1-oxopropan-2-yl Acetate (46)

Under argon atmosphere, to a solution of 11 (100 mg, 0.247 mmol) and triethylamine (0.07 mL, 0.5 mmol) in 10 mL of anhydrous DCM was added acetyl chloride (0.02 mL, 0.3 mmol) at ice-water bath. After stirring for 30 min, the temperature was raised to room temperature and the mixture stirred for 2 hours. The reaction mixture was washed with water, evaporated, dried over anhydrous MgSO₄, and evaporated to dryness. The mixture was purified with flash column chromatography as an eluent EtOAc/hexane (1/1, v/v) to produce target product as white solid.

Yield=86%;

MS (ESI) m/z 446.0 [M−H]−;

¹H NMR (CDCl₃, 400 MHz) δ 8.75 (bs, 1H, C(O)NH), 7.88 (s, 1H, ArH), 7.79-7.73 (m, 2H, ArH), 7.35 (dd, J=8.8, 4.2 Hz, 1H, ArH), 7.22 (dd, J=9.6, 2.6 Hz, 1H, ArH), 7.16 (d, J=2.6 Hz, 1H, ArH), 6.94 (m, 1H, ArH), 6.46 (d, J=3.2 Hz, 1H, ArH), 4.65 (d, J=14.8 Hz, 1H, CH₂), 4.33 (d, J=14.8 Hz, 1H, CH₂), 2.59 (s, 3H, OC(O)CH₃), 1.57 (s, 3H, CH₃);

¹⁹F NMR (CDCl₃, 400 MHz) δ −62.24, −124.54; assigned by 2D NMR as NOE and COSY.

### Example 4

### Synthesis of Indazole SARD Compound

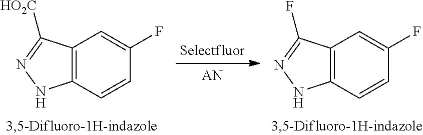 [SMILES: O=C(O)c1n[nH]c2ccc(F)cc12 ; Fc1ccc2[nH]nc(F)c2c1]

To a 50 mL round-bottle flask with a magnetic stirring bar were added Selectfluor® (872 mg, 2.0 mmol, 2.0 equiv), Li₂CO₃ (296 mg, 4.0 mmol, 4.0 equiv), dichloromethane (3.3 mL) and water (1.7 mL). Then carboxylic acid (1.0 mmol, 1.0 equiv) was added. The reaction mixture was stirred for 2 hours in ice bath. The reaction mixture was diluted with water (40 mL), followed by extracting with DCM (20 mL×2). The combined organic extracts were washed with brine, dried over anhydrous sodium sulfate and concentrated in vacuo. The crude product was purified by flash column chromatography (n-hexane:DCM=2:1) to afford the desired product.

Yield 48%;

MS (ESI) m/z 152.0 [M−H]−;

¹H NMR (CDCl₃, 400 MHz) δ 9.80 (bs, 1H, NH), 7.37 (dt, J=8.8, 2.4 Hz, 1H), 7.31 (dd, J=8.0, 1.6 Hz, 1H), 7.23 (td, J=8.8, 2.0 Hz, 1H);

¹⁹F NMR (CDCl₃) δ −121.46 (d, J_{F-F}=4.4 Hz), −133.92 (d, J_{F-F}=4.4 Hz).

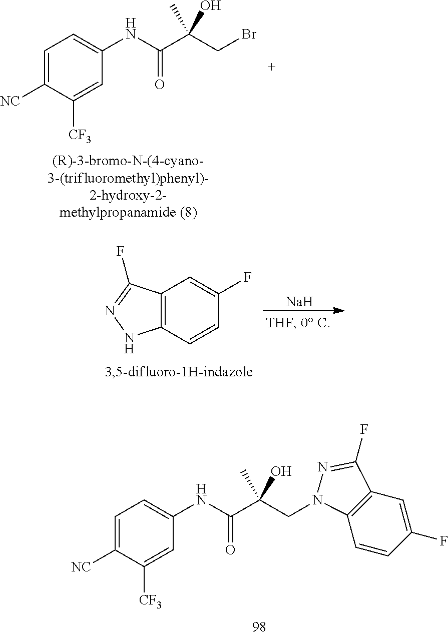

### (S)-N-(4-Cyano-3-(trifluoromethyl)phenyl)-3-(3,5-difluoro-1H-indazol-1-yl)-2-hydroxy-2-methylpropanamide (98)

To a dry, nitrogen-purged 50 mL round-bottom flask equipped with a dropping funnel under argon atmosphere, NaH of 60% dispersion in mineral oil (32 mg, 0.8 mmol) was added in 5 mL of anhydrous THF solvent in the flask at ice-water bath, and 3,5-difluoro-1H-indazole (60 mg, 0.41 mmol) was stirred 30 min at the ice-water bath. Into the flask, (R)-3-bromo-N-(4-cyano-3-(trifluoromethyl)phenyl)-2-hydroxy-2-methylpropanamide (8) (148 mg, 0.41 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=2/3 to produce 98 as white solid.

Yield=57%;

MS (ESI) m/z 423.17 [M−H]−; 447.21 [M+Na]+;

¹H NMR (CDCl₃, 400 MHz) δ 9.07 (bs, 1H, NH), 7.92 (s, 1H), 7.78 (d, J=8.6 Hz, 1H), 7.73 (d, J=8.6 Hz, 1H), 7.42 (d, J=8.4 Hz, 1H), 7.28 (m, 1H), 7.25 (m, 1H), 5.28 (bs, 1H, OH), 4.82 (d, J=14.0 Hz, 1H), 4.27 (d, J=14.0 Hz, 1H), 1.52 (s, 3H);

¹⁹F NMR (CDCl₃, 400 MHz) δ −61.27, −120.39, −131.15; assigned by 2D NMR as NOE and COSY.

### Example 5

### Synthesis of Benzotriazole SARD Compounds

### (S)-N-(4-Cyano-3-(trifluoromethyl)phenyl)-2-hydroxy-2-methyl-3-(5-(trifluoromethyl)-2H-benzo[d][1,2,3]triazol-2-yl)propanamide (C₁₉H₁₃F₆N₅O₂) (300)

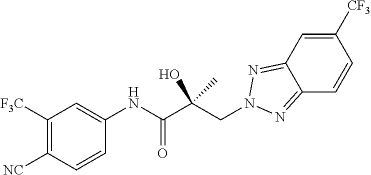 [SMILES: C[C@](O)(Cn1nc2ccc(C(F)(F)F)cc2n1)C(=O)Nc1ccc(C#N)c(C(F)(F)F)c1]

To a solution of 5-(trifluoromethyl)-1H-benzo[d][1,2,3]triazole (0.20 g, 0.0010688 mol) in anhydrous THF (5 mL), which was cooled in an ice water bath under an argon atmosphere, was added sodium hydride (60% dispersion in oil, 0.13 g, 0.0033134 mol). After addition, the resulting mixture was stirred for three hours. (R)-3-Bromo-N-(4-cyano-3-(trifluoromethyl)phenyl)-2-hydroxy-2-methylpropanamide (8) (0.375 g, 0.0010688 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using DCM and ethyl acetate (9:1) as eluent to afford 0.044 g (9%) of the titled compound as yellowish solid.

¹H NMR (400 MHz, DMSO-d₆) δ 10.33 (s, 1H, NH), 8.40 (s, 1H, ArH), 8.38 (s, 1H, ArH), 8.23 (d, J=8.4 Hz, 1H, ArH), 8.11 (d, J=8.4 Hz, 2H, ArH), 7.67 (d, J=8.6 Hz, 1H, ArH), 6.67 (s, 1H, OH), 5.24 (d, J=14.0 Hz, 1H, CH), 4.99 (d, J=14.0 Hz, 1H, CH), 1.55 (s, 3H, CH₃).

Mass (ESI, Negative): 456.25 [M−H]−; (ESI, Positive): 458.10[M+H]⁺.

### (S)-N-(4-Cyano-3-(trifluoromethyl)phenyl)-2-hydroxy-2-methyl-3-(5-(trifluoromethyl)-1H-benzo[d][1,2,3]triazol-1-yl)propanamide (C₁₉H₁₃F₆N₅O₂) (301)

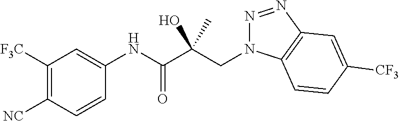 [SMILES: C[C@](O)(Cn1nnc2cc(C(F)(F)F)ccc21)C(=O)Nc1ccc(C#N)c(C(F)(F)F)c1]

To a solution of 5-(trifluoromethyl)-1H-benzo[d][1,2,3]triazole (0.20 g, 0.0010688 mol) in anhydrous THF (5 mL), which was cooled in an ice water bath under an argon atmosphere, was added sodium hydride (60% dispersion in oil, 0.13 g, 0.0033134 mol). After addition, the resulting mixture was stirred for three hours. (R)-3-Bromo-N-(4-cyano-3-(trifluoromethyl)phenyl)-2-hydroxy-2-methylpropanamide (8) (0.375 g, 0.0010688 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (3:1 to 2:1) as eluent to afford 0.025 g (5%) of the titled compound as yellowish solid.

¹H NMR (400 MHz, DMSO-d₆) δ 10.30 (s, 1H, NH), 8.39 (d, J=1.6 Hz, 1H, ArH), 8.33 (s, 1H, ArH), 8.25 (d, J=8.8 Hz, 1H, ArH), 8.12 (dd, J=8.8 Hz, J=2.0 Hz, 1H, ArH), 8.07 (d, J=8.4 Hz, 1H, ArH), 7.64 (dd, J=8.8 Hz, J=1.6 Hz, 1H, ArH), 6.64 (s, 1H, OH), 5.21 (d, J=14.4 Hz, 1H, CH), 5.01 (d, J=14.4 Hz, 1H, CH), 1.54 (s, 3H, CH₃).

Mass (ESI, Negative): 456.25 [M−H]−; (ESI, Positive): 458.10[M+H]⁺.

### (S)-N-(4-Cyano-3-(trifluoromethyl)phenyl)-2-hydroxy-2-methyl-3-(6-(trifluoromethyl)-1H-benzo[d][1,2,3]triazol-1-yl)propanamide (C₁₉H₁₃F₆N₅O₂) (302)

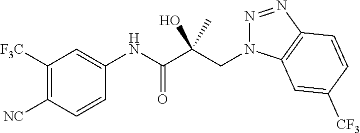 [SMILES: C[C@](O)(Cn1nnc2ccc(C(F)(F)F)cc21)C(=O)Nc1ccc(C#N)c(C(F)(F)F)c1]

To a solution of 5-(trifluoromethyl)-1H-benzo[d][1,2,3]triazole (0.20 g, 0.0010688 mol) in anhydrous THF (5 mL), which was cooled in an ice water bath under an argon atmosphere, was added sodium hydride (60% dispersion in oil, 0.13 g, 0.0033134 mol). After addition, the resulting mixture was stirred for three hours. (R)-3-Bromo-N-(4-cyano-3-(trifluoromethyl)phenyl)-2-hydroxy-2-methylpropanamide (8) (0.375 g, 0.0010688 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (3:1 to 2:1) as eluent to afford 0.023 g (5%) of the titled compound as yellowish solid.

¹H NMR (400 MHz, DMSO-d₆) δ 10.31 (s, 1H, NH), 8.50 (s, 1H, ArH), 8.34 (d, J=1.6 Hz, 1H, ArH), 8.18 (dd, J=8.8 Hz, J=2.0 Hz, 1H, ArH), 8.10 (d, J=8.8 Hz, 1H, ArH), 8.08 (d, J=8.4 Hz, 1H, ArH), 7.84 (dd, J=8.8 Hz, J=1.6 Hz, 1H, ArH), 6.49 (s, 1H, OH), 5.15 (d, J=14.4 Hz, 1H, CH), 4.97 (d, J=14.4 Hz, 1H, CH), 1.52 (s, 3H, CH₃).

Mass (ESI, Negative): 456.25 [M−H]−; (ESI, Positive): 458.10[M+H]⁺.

### (S)-N-(4-Cyano-3-(trifluoromethyl)phenyl)-2-hydroxy-2-methyl-3-(5-fluoro-2H-benzo[d][1,2,3]triazol-2-yl)propanamide (C₁₈H₁₃F₄N₅O₂) (303)

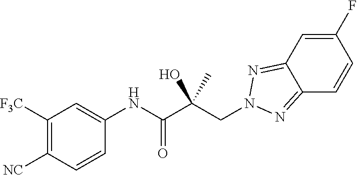 [SMILES: C[C@](O)(Cn1nc2ccc(F)cc2n1)C(=O)Nc1ccc(C#N)c(C(F)(F)F)c1]

To a solution of 5-fluoro-1H-benzo[d][1,2,3]triazole (0.20 g, 0.001459 mol) in anhydrous THF (5 mL), which was cooled in an ice water bath under an argon atmosphere, was added sodium hydride (60% dispersion in oil, 0.18 g, 0.004522 mol). After addition, the resulting mixture was stirred for three hours. (R)-3-Bromo-N-(4-cyano-3-(trifluoromethyl)phenyl)-2-hydroxy-2-methylpropanamide (8) (0.51 g, 0.001459 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (3:1 to 2:1) as eluent to afford 0.115 g (19.4%) of the titled compound as yellowish solid.

¹H NMR (400 MHz, DMSO-d₆) δ 10.35 (s, 1H, NH), 8.43 (s, 1H, ArH), 8.32 (d, J=8.2 Hz, 1H, ArH), 8.11 (d, J=8.2 Hz, 1H, ArH), 7.95-7.91 (m, 1H, ArH), 7.67 (d, J=8.8 Hz, 1H, ArH), 7.33-6-7.31 (m, 1H, ArH), 6.53 (s, 1H, OH), 5.14 (d, J=13.6 Hz, 1H, CH), 4.90 (d, J=13.6 Hz, 1H, CH), 1.53 (s, 3H, CH₃).

Mass (ESI, Positive): 430.09 [M+Na]+.

### (S)-N-(4-Cyano-3-(trifluoromethyl)phenyl)-2-hydroxy-2-methyl-3-(5-fluoro-1H-benzo[d][1,2,3]triazol-1-yl)propanamide (C₁₈H₁₃F₄N₅O₂) (304)

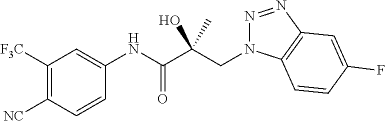 [SMILES: C[C@](O)(Cn1nnc2cc(F)ccc21)C(=O)Nc1ccc(C#N)c(C(F)(F)F)c1]

To a solution of 5-fluoro-1H-benzo[d][1,2,3]triazole (0.20 g, 0.001459 mol) in anhydrous THF (5 mL), which was cooled in an ice water bath under an argon atmosphere, was added sodium hydride (60% dispersion in oil, 0.18 g, 0.004522 mol). After addition, the resulting mixture was stirred for three hours. (R)-3-Bromo-N-(4-cyano-3-(trifluoromethyl)phenyl)-2-hydroxy-2-methylpropanamide (8) (0.51 g, 0.001459 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (3:1 to 2:1) as eluent to afford 0.075 g (12.6%) of the titled compound as yellowish solid.

¹H NMR (400 MHz, DMSO-d₆) δ 10.35 (s, 1H, NH), 8.40 (s, 1H, ArH), 8.19 (d, J=8.4 Hz, 1H, ArH), 8.10 (d, J=8.0 Hz, 1H, ArH), 8.07-8.04 (m, 1H, ArH), 7.70 (d, J=8.2 Hz, 1H, ArH), 7.28-6-7.23 (m, 1H, ArH), 6.45 (s, 1H, OH), 5.05 (d, J=14.4 Hz, 1H, CH), 4.87 (d, J=14.4 Hz, 1H, CH), 1.50 (s, 3H, CH₃).

Mass (ESI, Positive): 430.09 [M+Na]+.

### (S)-N-(4-Cyano-3-(trifluoromethyl)phenyl)-2-hydroxy-2-methyl-3-(6-fluoro-1H-benzo[d][1,2,3]triazol-1-yl)propanamide (C₁₈H₁₃F₄N₅O₂) (305)

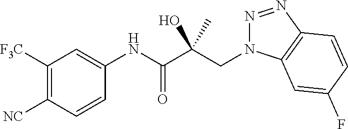 [SMILES: C[C@](O)(Cn1nnc2ccc(F)cc21)C(=O)Nc1ccc(C#N)c(C(F)(F)F)c1]

To a solution of 5-fluoro-1H-benzo[d][1,2,3]triazole (0.20 g, 0.001459 mol) in anhydrous THF (5 mL), which was cooled in an ice water bath under an argon atmosphere, was added sodium hydride (60% dispersion in oil, 0.18 g, 0.004522 mol). After addition, the resulting mixture was stirred for three hours. (R)-3-Bromo-N-(4-cyano-3-(trifluoromethyl)phenyl)-2-hydroxy-2-methylpropanamide (8) (0.51 g, 0.001459 mol) was added to above solution, and the resulting reaction mixture was allowed to stir overnight at room temperature under argon. The reaction was quenched by water, and extracted with ethyl acetate. The organic layer was washed with brine, dried with MgSO₄, filtered, and concentrated under vacuum. The product was purified by a silica gel column using hexanes and ethyl acetate (3:1 to 2:1) as eluent to afford 0.052 g (8.8%) of the titled compound as yellowish solid.

¹H NMR (400 MHz, DMSO-d₆) δ 10.35 (s, 1H, NH), 8.38 (s, 1H, ArH), 8.20 (d, J=8.8 Hz, 1H, ArH), 8.10 (d, J=8.4 Hz, 1H, ArH), 7.92-7.89 (m, 1H, ArH), 7.84 (d, J=8.8 Hz, 1H, ArH), 7.46-7.42 (m, 1H, ArH), 6.46 (s, 1H, OH), 5.08 (d, J=14.4 Hz, 1H, CH), 4.90 (d, J=14.4 Hz, 1H, CH), 1.49 (s, 3H, CH₃).

Mass (ESI, Positive): 430.09 [M+Na]+.

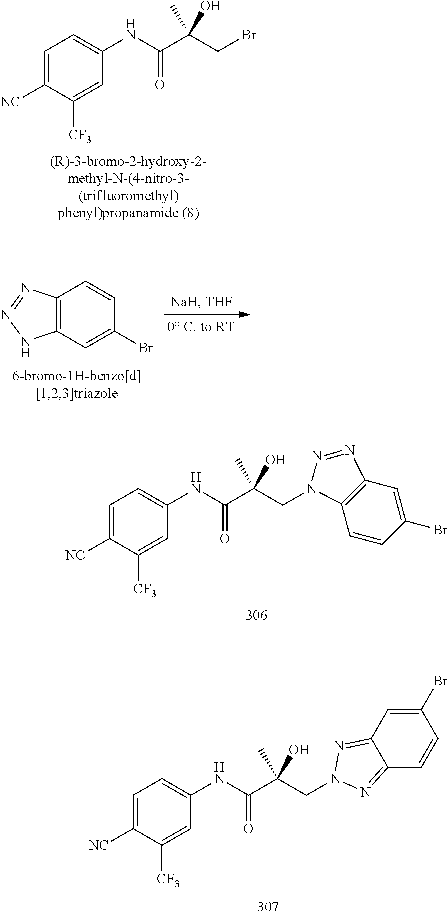 [SMILES: C[C@](O)(CBr)C(=O)Nc1ccc(C#N)c(C(F)(F)F)c1 ; Brc1ccc2nn[nH]c2c1 ; C[C@](O)(Cn1nnc2cc(Br)ccc21)C(=O)Nc1ccc(C#N)c(C(F)(F)F)c1 ; C[C@](O)(Cn1nc2ccc(Br)cc2n1)C(=O)Nc1ccc(C#N)c(C(F)(F)F)c1]

### (S)-3-(5-Bromo-1H-benzo[d][1,2,3]triazol-1-yl)-N-(4-cyano-3-(trifluoromethyl)phenyl)-2-hydroxy-2-methylpropanamide (306)

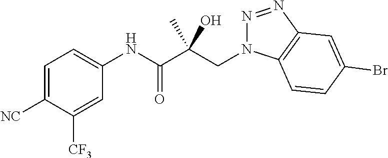 [SMILES: C[C@](O)(Cn1nnc2cc(Br)ccc21)C(=O)Nc1ccc(C#N)c(C(F)(F)F)c1]

To a dry, nitrogen-purged 100 mL round-bottom flask equipped with a dropping funnel under argon atmosphere, NaH of 60% dispersion in mineral oil (260 mg, 6.5 mmol) was added in 30 mL of anhydrous THF solvent in the flask at ice-water bath, and 6-bromo-1H-benzo[d][1,2,3]triazole (514 mg, 2.6 mmol) was stirred in over 30 min at the ice-water bath. Into the flask, the solution of (R)-3-bromo-2-hydroxy-2-methyl-N-(4-cyano-3-(trifluoromethyl)phenyl)propanamide (8) (911 mg, 2.6 mmol) in 5 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=1/2 to produce designed compounds (Yield=65%: 42% for 306 and 23% of 307) as yellowish solid.

### (S)-3-(5-Bromo-1H-benzo[d][1,2,3]triazol-1-yl)-N-(4-cyano-3-(trifluoromethyl)phenyl)-2-hydroxy-2-methylpropanamide (306)

MS (ESI) m/z 467.81 [M−H]−; 492.00 [M+Na]+;

¹H NMR (400 MHz, CDCl₃) δ 9.10 (bs, 1H, NH), 8.04 (s, 1H), 8.02 (s, 1H), 7.90 (d, J=8.0 Hz, 1H), 7.76 (d, J=8.0 Hz, 1H), 7.74 (d, J=8.8 Hz, 1H), 7.49 (d, J=8.8 Hz, 1H), 5.48 (s, 1H, OH), 5.26 (d, J=13.6 Hz, 1H), 4.94 (d, J=13.6 Hz, 1H), 1.54 (s, 3H);

¹⁹F NMR (CDCl3, decoupled) δ −62.19.

### (S)-3-(5-Bromo-2H-benzo[d][1,2,3]triazol-2-yl)-N-(4-cyano-3-(trifluoromethyl)phenyl)-2-hydroxy-2-methylpropanamide (307)

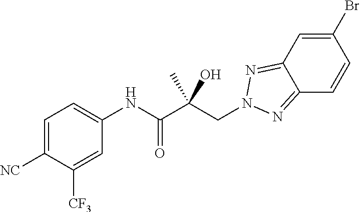 [SMILES: C[C@](O)(Cn1nc2ccc(Br)cc2n1)C(=O)Nc1ccc(C#N)c(C(F)(F)F)c1]

MS (ESI) m/z 467.84 [M−H]−;

¹H NMR (400 MHz, CDCl₃) δ 8.97 (bs, 1H, NH), 8.15 (s, 1H), 7.92 (s, 1H), 7.75 (m, 2H), 7.62 (d, J=9.0 Hz, 1H), 7.53 (d, J=9.0 Hz, 1H), 5.16 (d, J=14.2 Hz, 1H), 4.79 (s, 1H, OH), 4.78 (d, J=14.2 Hz, 1H), 1.65 (s, 3H);

¹⁹F NMR (CDCl₃, decoupled) 6-62.26.

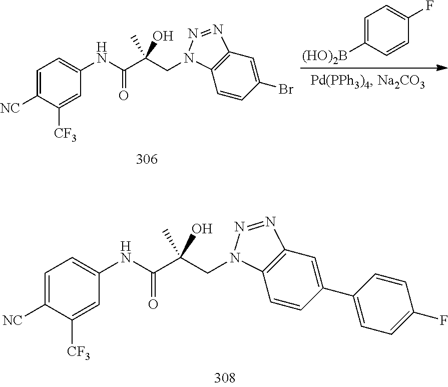 [SMILES: C[C@](O)(Cn1nnc2cc(Br)ccc21)C(=O)Nc1ccc(C#N)c(C(F)(F)F)c1 ; OB(O)c1ccc(F)cc1 ; C[C@](O)(Cn1nnc2cc(-c3ccc(F)cc3)ccc21)C(=O)Nc1ccc(C#N)c(C(F)(F)F)c1]

### (S)-N-(4-Cyano-3-(trifluoromethyl)phenyl)-3-(5-(4-fluorophenyl)-1H-benzo[d][1,2,3]triazol-1-yl)-2-hydroxy-2-methylpropanamide (308)

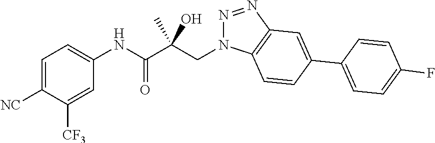 [SMILES: C[C@](O)(Cn1nnc2cc(-c3ccc(F)cc3)ccc21)C(=O)Nc1ccc(C#N)c(C(F)(F)F)c1]

A mixture of 306 (150 mg, 0.32 mmol), tetrakis(triphenylphosphine)palladium (0) (13 mg, 12 mmol) and trimethoxyboric acid (50 mg, 0.35 mmol) in THF/MeOH (1/1 mL) with sodium carbonate (82 mg, 7.69 mmol) in ethanol/water (5 mL/1 mL) were heated to reflux overnight. The mixture was cooled down to be concentrated under reduced pressure and poured into EtOAc, which was washed with water and dried over MgSO₄, concentrated, purified by silica gel chromatography (EtOAc/n-hexane=2:3) to afford 308 as a yellow solid.

Yield=90%;

MS (ESI) m/z 482.25 [M−H]−;

¹H NMR (400 MHz, CDCl₃) δ 9.12 (bs, 1H, NH), 8.02 (s, 1H), 7.96 (s, 1H), 7.92 (d, J=9.2 Hz, 1H), 7.76 (d, J=8.4 Hz, 1H), 7.64 (d, J=9.2 Hz, 1H), 7.59 (dd, J=7.6, 5.2 Hz, 2H), 7.17 (t, J=8.4 Hz, 2H), 5.72 (s, 1H, OH), 5.28 (d, J=14.0 Hz, 1H), 4.97 (d, J=14.0 Hz, 1H), 1.55 (s, 3H);

¹⁹F NMR (CDCl₃, decoupled) 6-62.20, −114.49.

### Example 6

### Synthesis of Pyrazole SARD Compounds

### (S)-3-(4-Bromo-1H-pyrazol-1-yl)-N-(4-cyano-3-(trifluoromethyl)phenyl)-2-hydroxy-2-methylpropanamide (C₁₅H₁₂BrF3N₄O₂) (1050)

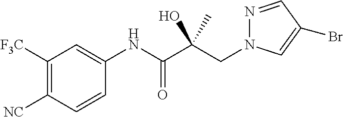 [SMILES: C[C@](O)(Cn1cc(Br)cn1)C(=O)Nc1ccc(C#N)c(C(F)(F)F)c1]

To a solution of 4-bromo-1H-pyrazole (0.20 g, 0.0013608 mol) in anhydrous THF (5 mL), which was cooled in an ice water bath under an argon atmosphere, was added sodium hydride (60% dispersion in oil, 0.16 g, 0.0040827 mol). After addition, the resulting mixture was stirred for three hours. (R)-3-Bromo-N-(4-cyano-3-(trifluoromethyl)phenyl)-2-hydroxy-2-methylpropanamide (8) (0.478 g, 0.001608 mol) was added to the above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using DCM and ethyl acetate (19:1) as eluent to afford 0.47 g (79.6%) of the titled compound as white foam.

¹H NMR (400 MHz, CDCl₃) δ 9.08 (s, 1H, NH), 8.00 (d, J=2.0 Hz, 1H, ArH), 7.87 (dd, J=8.4 Hz, J=2.0 Hz, 1H, ArH), 7.79 (d, J=8.4 Hz, 1H, ArH), 7.49 (s, 1H, Pyrazole-H), 7.47 (s, 1H, Pyrazole-H), 5.92 (s, 1H, OH), 4.64 (d, J=14.0 Hz, 1H, CH), 4.24 (d, J=14.0 Hz, 1H, CH), 1.47 (s, 3H, CH₃).

Mass (ESI, Negative): 371.68 [M−H]−; (ESI, Positive): 440.94 [M+Na]+.

### (R)-3-Bromo-N-(4-cyano-2-iodo-5-(trifluoromethyl)phenyl)-2-hydroxy-2-methylpropanamide (C₁₂H₉BrF₃IN₂O₂) (1051)

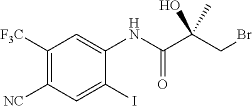 [SMILES: C[C@](O)(CBr)C(=O)Nc1cc(C(F)(F)F)c(C#N)cc1I]

3-Bromo-2-methyl-2-hydroxypropanoic acid (4) (0.50 g, 0.00273224 mol) was reacted with thionyl chloride (0.39 g, 0.0032787 mol), trimethylamine (0.36 g, 0.0035519 mol), and 4-amino-5-iodo-2-(trifluoromethyl)benzonitrile (0.81 g, 0.0025956 mol) to afford the titled compound. The product was purified by a silica gel column using DCM and ethyl acetate (9:1) as eluent to afford 0.80 g (64.6%) of the titled compound as a light brown solid.

¹H NMR (400 MHz, CDCl₃) δ 9.53 (s, 1H, NH), 8.92 (s, 1H, ArH), 8.24 (s, 1H, ArH), 7.26 (s, 1H, OH), 4.04 (d, J=10.4 Hz, 1H, CH), 3.62 (d, J=10.4 Hz, 1H, CH), 1.67 (s, 3H, CH₃).

Mass (ESI, Positive): 479.25[M+H]⁺.

### (S)-N-(4-Cyano-2-iodo-5-(trifluoromethyl)phenyl)-3-(4-fluoro-1H-pyrazol-1-yl)-2-hydroxy-2-methylpropanamide (C₁₅H₁₁F₄IN₄O₂) (1052)

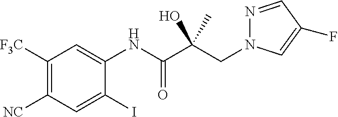 [SMILES: C[C@](O)(Cn1cc(F)cn1)C(=O)Nc1cc(C(F)(F)F)c(C#N)cc1I]

To a solution of 4-fluoro-1H-pyrazole (0.09 g, 0.001048 mol) in anhydrous THF (5 mL), which was cooled in an ice water bath under an argon atmosphere, was added sodium hydride (60% dispersion in oil, 0.15 g, 0.003669 mol). After addition, the resulting mixture was stirred for three hours. (R)-3-Bromo-N-(4-cyano-2-iodo-5-(trifluoromethyl)phenyl)-2-hydroxy-2-methylpropanamide (0.50 g, 0.001048 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using hexanes and ethyl acetate (2:1 to 1:1) as eluent to afford 0.32 g (64%) of the titled compound as a white solid.

¹H NMR (400 MHz, CDCl₃) δ 9.60 (s, 1H, NH), 8.76 (s, 1H, ArH), 8.69 (s, 1H, ArH), 7.76 (d, J=4.8 Hz, 1H, Pyrazole-H), 7.36 (d, J=4.4 Hz, 1H, Pyrazole-H), 6.85 (s, 1H, OH), 4.39 (d, J=14.0 Hz, 1H, CH), 4.20 (d, J=14.0 Hz, 1H, CH), 1.41 (s, 3H, CH₃).

Mass (ESI, Negative): 481.00 [M−H]⁻;

### (S)-N-(4-Cyano-3-(trifluoromethyl)phenyl)-3-(5-(4-fluorophenyl)-1H-tetrazol-1-yl)-2-hydroxy-2-methylpropanamide (C₁₉H₁₄F₄N₆O₂) (1053)

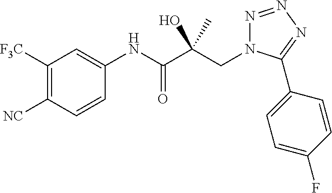 [SMILES: C[C@](O)(Cn1nnnc1-c1ccc(F)cc1)C(=O)Nc1ccc(C#N)c(C(F)(F)F)c1]

To a solution of 5-(4-fluorophenyl)-1H-tetrazole (0.20 g, 0.001219 mol) in anhydrous THF (5 mL), which was cooled in an ice water bath under an argon atmosphere, was added sodium hydride (60% dispersion in oil, 0.17 g, 0.004265 mol). After addition, the resulting mixture was stirred for three hours. (R)-3-Bromo-N-(4-cyano-3-(trifluoromethyl)phenyl)-2-hydroxy-2-methylpropanamide (8) (0.43 g, 0.001219 mol) was added to above solution, and the resulting reaction mixture was allowed to stir 2 days at room temperature under argon. The reaction was quenched by water and extracted with ethyl acetate. The organic layer was washed with brine, dried with MgSO₄, filtered, and concentrated under vacuum. The product was purified by a silica gel column using DCM and ethyl acetate (9:1) as eluent to afford 0.053 g (10%) of the titled compound as a yellowish solid.

¹H NMR (400 MHz, CDCl₃) δ 10.39 (s, 1H, NH), 8.44 (s, 1H, ArH), 8.26 (d, J=8.2 Hz, 1H, ArH), 8.10 (d, J=8.2 Hz, 1H, ArH), 7.93-7.89 (m, 2H, ArH), 7.30 (t, J=8.2 Hz, 2H, ArH), 6.64 (s, 1H, OH), 5.09 (d, J=14.0 Hz, 1H, CH), 4.92 (d, J=14.0 Hz, 1H, CH), 1.55 (s, 3H, CH₃).

Mass (ESI, Negative): 433.17 [M−H]⁻.

### (S)-N-(4-Cyano-3-(trifluoromethyl)phenyl)-2-hydroxy-3-(4-methoxy-1H-pyrazol-1-yl)-2-methylpropanamide (C₁₆H₁₅F₃N₄O₃) (1054)

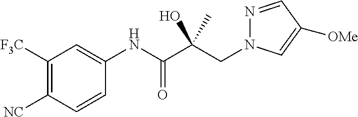 [SMILES: COc1cnn(C[C@](C)(O)C(=O)Nc2ccc(C#N)c(C(F)(F)F)c2)c1]

To a solution of 4-methoxy-1H-pyrazole (0.12 g, 0.001233 mol) in anhydrous THF (5 mL), which was cooled in an ice water bath under an argon atmosphere, was added sodium hydride (60% dispersion in oil, 0.17 g, 0.004281 mol). After addition, the resulting mixture was stirred for three hours. (R)-3-Bromo-N-(4-cyano-3-(trifluoromethyl)phenyl)-2-hydroxy-2-methylpropanamide (8) (0.43 g, 0.001233 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using DCM and ethyl acetate (9:1) as eluent to afford 0.30 g (60%) of the titled compound as a white solid.

¹H NMR (400 MHz, DMSO-d₆) δ 10.38 (s, 1H, NH), 8.46 (d, J=2.0 Hz, 1H, ArH), 8.24 (dd, J=8.2 Hz, J=2.0 Hz, 1H, ArH), 8.10 (d, J=8.2 Hz, 1H, ArH), 7.35 (d, J=0.8 Hz, 1H, Pyrazole-H), 7.15 (d, J=0.8 Hz, 1H, Pyrazole-H), 6.25 (s, 1H, OH), 4.35 (d, J=14.0 Hz, 1H, CH), 4.18 (d, J=14.0 Hz, 1H, CH), 3.61 (s, 3H, CH₃), 1.36 (s, 3H, CH₃).

HRMS [C₁₆H₁₆F₃N₄O₃+]: calcd 369.1175, found 369.1182[M+H]⁺. Purity: 99.28% (HPLC).

### (S)-N-(4-Cyano-3-(trifluoromethyl)phenyl)-2-hydroxy-2-methyl-3-(4-methyl-1H-pyrazol-1-yl)propanamide (C₁₆H₁₅F₃N₄O₂) (1055)

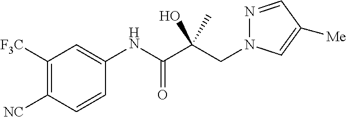 [SMILES: Cc1cnn(C[C@](C)(O)C(=O)Nc2ccc(C#N)c(C(F)(F)F)c2)c1]

To a solution of 4-methyl-1H-pyrazole (0.10 g, 0.001218 mol) in anhydrous THF (5 mL), which was cooled in an ice water bath under an argon atmosphere, was added sodium hydride (60% dispersion in oil, 0.17 g, 0.004263 mol). After addition, the resulting mixture was stirred for three hours. (R)-3-Bromo-N-(4-cyano-3-(trifluoromethyl)phenyl)-2-hydroxy-2-methylpropanamide (8) (0.428 g, 0.001218 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using DCM and ethyl acetate (19:1) as eluent to afford 0.28 g (66%) of the titled compound as a white solid.

¹H NMR (400 MHz, DMSO-d₆) δ 10.38 (s, 1H, NH), 8.46 (d, J=2.0 Hz, 1H, ArH), 8.23 (dd, J=8.8 Hz, J=2.0 Hz, 1H, ArH), 8.10 (d, J=8.8 Hz, 1H, ArH), 7.41 (s, 1H, Pyrazole-H), 7.17 (s, 1H, Pyrazole-H), 6.24 (s, 1H, OH), 4.40 (d, J=14.0 Hz, 1H, CH), 4.22 (d, J=14.0 Hz, 1H, CH), 1.97 (s, 3H, CH₃), 1.36 (s, 3H, CH₃).

HRMS [C₁₆H₁₆F₃N₄O₂+]: calcd 353.1225, found 353.1232[M+H]⁺. Purity: 99.75% (HPLC).

### N-(4-Cyano-3-(trifluoromethyl)phenyl)-2-methyloxirane-2-carboxamide (C₁₂H₉F₃N₂O₂) (1056)

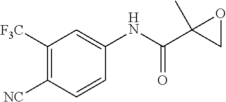 [SMILES: CC1(C(=O)Nc2ccc(C#N)c(C(F)(F)F)c2)CO1]

2-Methyloxirane-2-carboxylic acid (1.00 g, 0.009892 mol) was reacted with thionyl chloride (1.41 g, 0.011871 mol), trimethylamine (1.30 g, 0.01286 mol), and 4-amino-2-(trifluoromethyl)benzonitrile (1.84 g, 0.009892 mol) to afford the titled compound.

The product was purified by a silica gel column using DCM and ethyl acetate (19:1) as eluent to afford 1.52 g (57%) of the titled compound as a yellowish solid.

¹H NMR (400 MHz, DMSO-d₆) δ 10.54 (s, 1H, NH), 8.55 (d, J=1.6-2.0 Hz, 1H, ArH), 8.32 (dd, J=8.8 Hz, J=2.0 Hz, 1H, ArH), 8.12 (d, J=8.8 Hz, 1H, ArH), 6.39 (s, 1H, OH), 3.94 (d, J=11.2 Hz, 1H, CH), 3.70 (d, J=11.2 Hz, 1H, CH), 1.44 (s, 3H, CH₃).

Mass (ESI, Negative): [M−H]⁻; (ESI, Positive): [M+Na]⁺.

### N-(4-Cyano-3-(trifluoromethyl)phenyl)-3-(4-fluoro-1H-pyrazol-1-yl)-2-hydroxy-2-methylpropanamide (C₅H₁₂F₄N₄O₂) (1057) (Racemate of 1002)

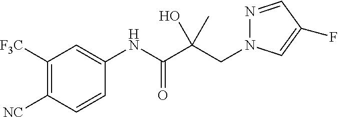 [SMILES: CC(O)(Cn1cc(F)cn1)C(=O)Nc1ccc(C#N)c(C(F)(F)F)c1]

To a solution of 4-fluoro-pyrazole (0.10 g, 0.001162 mol) in anhydrous THF (10 mL), which was cooled in an ice water bath under an argon atmosphere, was added sodium hydride (60% dispersion in oil, 0.14 g, 0.003486 mol). After addition, the resulting mixture was stirred for three hours. N-(4-Cyano-3-(trifluoromethyl)phenyl)-2-methyloxirane-2-carboxamide (1056) (0.31 g, 0.001162 mol) was added to above solution, and the resulting reaction mixture was allowed to stir overnight at room temperature under argon. The reaction was quenched by water, extracted with ethyl acetate. The organic layer was washed with brine, dried with MgSO₄, filtered, and concentrated under vacuum.

The product was purified by a silica gel column using hexanes and ethyl acetate (2:1 to 1:1) as eluent to afford 0.37 g (90%) of the titled compound as a yellowish solid.

¹H NMR (400 MHz, DMSO-d₆) δ 10.38 (s, 1H, NH), 8.47 (d, J=2.0 Hz, 1H, ArH), 8.24 (dd, J=8.8 Hz, J=2.0 Hz, 1H, ArH), 8.10 (d, J=8.8 Hz, 1H, ArH), 7.74 (d, J=4.4 Hz, 1H, Pyrazole-H), 7.41 (d, J=4.0 Hz, 1H, Pyrazole-H), 6.31 (s, 1H, OH), 4.39 (d, J=14.0 Hz, 1H, CH), 4.21 (d, J=14.4 Hz, 1H, CH), 1.34 (s, 3H, CH₃).

Mass (ESI, Negative): [M−H]⁻; (ESI, Positive): [M+Na]⁺.

### (S)-3-Bromo-N-(4-cyano-3-(trifluoromethyl)phenyl)-2-hydroxy-2-methylpropanamide (C₁₂H₉F₃N₂O₂)

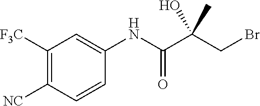 [SMILES: C[C@@](O)(CBr)C(=O)Nc1ccc(C#N)c(C(F)(F)F)c1]

(S)-3-Bromo-2-hydroxy-2-methylpropanoic acid (1.00 g, 0.0054645 mol) reacted with thionyl chloride (0.78 g, 0.0065574 mol), trimethylamine (0.72 g, 0.0071038 mol), and 4-amino-2-(trifluoromethyl)benzonitrile (1.02 g, 0.0054645 mol) to afford the titled compound.

The product was purified by a silica gel column using DCM and ethyl acetate (19:1) as eluent to afford 1.75 g (90%) of the titled compound as a yellowish solid.

Mass (ESI, Positive): 351.08 [M+Na]⁺.

### (R)-N-(4-Cyano-3-(trifluoromethyl)phenyl)-3-(4-fluoro-1H-pyrazol-1-yl)-2-hydroxy-2-methylpropanamide (C₅H₁₂F₄N₄O₂) (R-isomer of 1002)

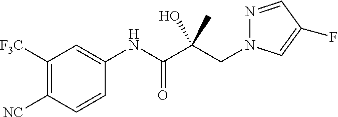 [SMILES: C[C@@](O)(Cn1cc(F)cn1)C(=O)Nc1ccc(C#N)c(C(F)(F)F)c1]

To a solution of 4-fluoro-pyrazole (0.10 g, 0.001162 mol) in anhydrous THF (10 mL), which was cooled in an ice water bath under an argon atmosphere, was added sodium hydride (60% dispersion in oil, 0.16 g, 0.0040665 mol). After addition, the resulting mixture was stirred for three hours. (S)-3-Bromo-N-(4-cyano-3-(trifluoromethyl)phenyl)-2-hydroxy-2-methylpropanamide (0.41 g, 0.001162 mol) was added to above solution, and the resulting reaction mixture was allowed to stir overnight at room temperature under argon. The reaction was quenched by water, extracted with ethyl acetate. The organic layer was washed with brine, dried with MgSO₄, filtered, and concentrated under vacuum.

The product was purified by a silica gel column using hexanes and ethyl acetate (2:1 to 1:1) as eluent to afford 0.27 g (64%) of the titled compound as yellowish solid.

¹H NMR (400 MHz, DMSO-d₆) δ 10.38 (s, 1H, NH), 8.47 (d, J=1.6-2.0 Hz, 1H, ArH), 8.24 (dd, J=8.4 Hz, J=2.0 Hz, 1H, ArH), 8.10 (d, J=8.4 Hz, 1H, ArH), 7.74 (d, J=4.4 Hz, 1H, Pyrazole-H), 7.41 (d, J=4.4 Hz, 1H, Pyrazole-H), 6.31 (s, 1H, OH), 4.39 (d, J=14.0 Hz, 1H, CH), 4.21 (d, J=14.4 Hz, 1H, CH), 1.34 (s, 3H, CH₃).

Mass (ESI, Positive): 357.11 [M+Na]⁺.

### (S)-3-(4-Bromo-3-fluoro-1H-pyrazol-1-yl)-N-(4-cyano-3-(trifluoromethyl)phenyl)-2-hydroxy-2-methylpropanamide (C₁₅H₁₁BrF₄N₄O₂) (1058)

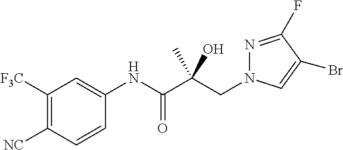 [SMILES: C[C@](O)(Cn1cc(Br)c(F)n1)C(=O)Nc1ccc(C#N)c(C(F)(F)F)c1]

To a solution of 4-bromo-3-fluoro-1H-pyrazole (0.30 g, 0.001819 mol) in anhydrous THF (10 mL), which was cooled in an ice water bath under an argon atmosphere, was added sodium hydride (60% dispersion in oil, 0.26 g, 0.006365 mol). After addition, the resulting mixture was stirred for three hours. (R)-3-Bromo-N-(4-cyano-3-(trifluoromethyl)phenyl)-2-hydroxy-2-methylpropanamide (8) (0.64 g, 0.001819 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using ethyl acetate and hexanes (2:1) as eluent to afford 0.34 g (34%) of the titled compound as a pinkish solid.

¹H NMR (400 MHz, DMSO-d₆) δ 10.38 (s, 1H, NH), 8.45 (d, J=2.0-1.6 Hz, 1H, ArH), 8.23 (dd, J=8.2 Hz, J=2.0 Hz, 1H, ArH), 8.11 (d, J=8.2 Hz, 1H, ArH), 7.82 (d, J=2.0 Hz, 1H, Pyrazole-H), 6.35 (s, 1H, OH), 4.35 (d, J=14.0 Hz, 1H, CH), 4.04 (d, J=14.0 Hz, 1H, CH), 1.37 (s, 3H, CH₃).

HRMS [C₁₅H₁₂BrF₄N₄O₂⁺]: calcd 435.0080, found 435.0080[M+H]⁺. Purity: 96.98% (HPLC).

### (S)-N-(4-Cyano-3-(trifluoromethyl)phenyl)-3-(3-fluoro-4-(4-fluorophenyl)-1H-pyrazol-1-yl)-2-hydroxy-2-methylpropanamide (C₂₁H₁₅F₅N₄O₂) (1059)

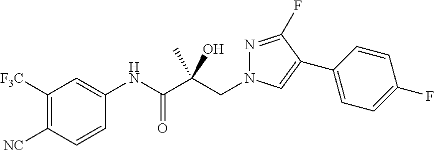 [SMILES: C[C@](O)(Cn1cc(-c2ccc(F)cc2)c(F)n1)C(=O)Nc1ccc(C#N)c(C(F)(F)F)c1]

The mixture of (S)-3-(4-bromo-3-fluoro-1H-pyrazol-1-yl)-N-(4-cyano-3-(trifluoromethyl)phenyl)-2-hydroxy-2-methylpropanamide (1058, 0.20 g, 0.4596 mmol), 4-fluoro boronic acid (77 mg, 0.5515 mmol), Pd(II)(OAc)₂ (2-3 mg, 0.009192 mmol), PPh₃ (7-8 mg, 0.02758 mmol), and K₂CO₃ (0.13 g, 0.965 mmol) in the mixture of ACN (4-5 mL) and H₂O (2-3 mL) was degassed and refilled with argon three times. The resulting reacting mixture was heated at reflux for 3 hours under argon. The product was purified by a silica gel column using hexanes and ethyl acetate (2:1 to 1:1) as eluent to afford 0.51 mg (25%) of the titled compound as a yellowish solid.

¹H NMR (400 MHz, cCDCl₃) δ 9.12 (s, 1H, NH), 8.06 (d, J=1.6 Hz, 1H, ArH), 7.85 (dd, J=8.2 Hz, J=1.6 Hz, 1H, ArH), 7.77 (d, J=8.2 Hz, 1H, ArH), 7.51 (d, J=3.0 Hz, 1H, Pyrazole-H), 7.43-7.40 (m, 2H, ArH), 7.08-7.04 (m, 2H, ArH), 4.57 (d, J=10.5 Hz, 1H, CH), 4.7 (d, J=10.5 Hz, 1H, CH), 1.26 (s, 3H, CH₃).

HRMS [C₂₁H₁₆F₅N₄O₂⁺]: calcd 451.1193, found 451.1196[M+H]⁺. Purity: % (HPLC).

### (S)-3-(3-Bromo-4-cyano-1H-pyrazol-1-yl)-N-(4-cyano-3-(trifluoromethyl)phenyl)-2-hydroxy-2-methylpropanamide (C₁₆H₁₁BrF₃N₅O₂) (1060)

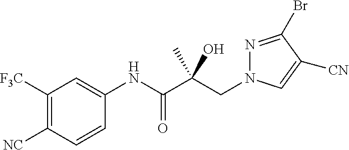 [SMILES: C[C@](O)(Cn1cc(C#N)c(Br)n1)C(=O)Nc1ccc(C#N)c(C(F)(F)F)c1]

To a solution of 3-bromo-4-cyano-1H-pyrazole (0.20 g, 0.0011629 mol) in anhydrous THF (10 mL), which was cooled in an ice water bath under an argon atmosphere, was added sodium hydride (60% dispersion in oil, 0.163 g, 0.00407 mol). After addition, the resulting mixture was stirred for three hours. (R)-3-Bromo-N-(4-cyano-3-(trifluoromethyl)phenyl)-2-hydroxy-2-methylpropanamide (8) (0.41 g, 0.0011629 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using ethyl acetate and hexanes (2:1) as eluent to afford 0.10 g (20%) of the titled compound as a off-white solid.

¹H NMR (400 MHz, DMSO-d₆) δ 10.32 (s, 1H, NH), 8.40 (s 1H, Pyrazole-H), 8.41 (s, 1H, ArH), 8.20 (d, J=8.4 Hz, 1H, ArH), 8.11 (d, J=8.4 Hz, 1H, ArH), 6.47 (s, 1H, OH), 4.52 (d, J=13.6 Hz, 1H, CH), 4.33 (d, J=13.6 Hz, 1H, CH), 1.41 (s, 3H, CH₃).

HRMS [C₁₆H₁₂BrF₃N₅O₂⁺]: calcd 442.0126, found 442.0109[M+H]⁺. Purity: 98.84% (HPLC).

### (S)-3-(3-Chloro-4-methyl-1H-pyrazol-1-yl)-N-(4-cyano-3-(trifluoromethyl)phenyl)-2-hydroxy-2-methylpropanamide (C₁₆H₁₄ClF₃N₄O₂)(1061)

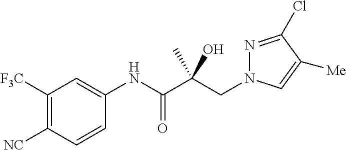 [SMILES: Cc1cn(C[C@](C)(O)C(=O)Nc2ccc(C#N)c(C(F)(F)F)c2)nc1Cl]

To a solution of 3-chloro-4-methyl-1H-pyrazole (0.15 g, 0.001287 mol) in anhydrous THF (10 mL), which was cooled in an ice water bath under an argon atmosphere, was added sodium hydride (60% dispersion in oil, 0.18 g, 0.0045045 mol). After addition, the resulting mixture was stirred for three hours. (R)-3-Bromo-N-(4-cyano-3-(trifluoromethyl)phenyl)-2-hydroxy-2-methylpropanamide (8) (0.45 g, 0.001287 mol) was added to above solution, and the resulting reaction mixture was allowed to stir overnight at room temperature under argon. The reaction was quenched by water and extracted with ethyl acetate. The organic layer was washed with brine, dried with MgSO₄, filtered, and concentrated under vacuum. The product was purified by a silica gel column using DCM and ethyl acetate (98:2 to 95:5) as eluent to afford 0.27 g (54%) of the titled compound as a white solid.

¹H NMR (400 MHz, DMSO-d₆) δ 10.33 (s, 1H, NH), 8.42 (d, J=0.8 Hz, 1H, ArH), 8.21 (dd, J=8.4 Hz, J=0.8 Hz, 1H, ArH), 8.10 (d, J=8.2 Hz, 1H, ArH), 7.50 (s 1H, Pyrazole-H), 6.29 (s, 1H, OH), 4.36 (d, J=14.4 Hz, 1H, CH), 4.18 (d, J=14.4 Hz, 1H, CH), 1.91 (s, 3H, CH₃), 1.35 (s, 3H, CH₃).

HRMS [C₁₆H₁₅ClF₃N₄O₂⁺]: calcd 387.0836, found 387.0839[M+H]⁺. Purity: 97.07% (HPLC).

### (S)-3-(4-Fluoro-1H-pyrazol-1-yl)-2-hydroxy-2-methyl-N-(4-nitro-3-(trifluoromethyl)phenyl)propanamide (1062)

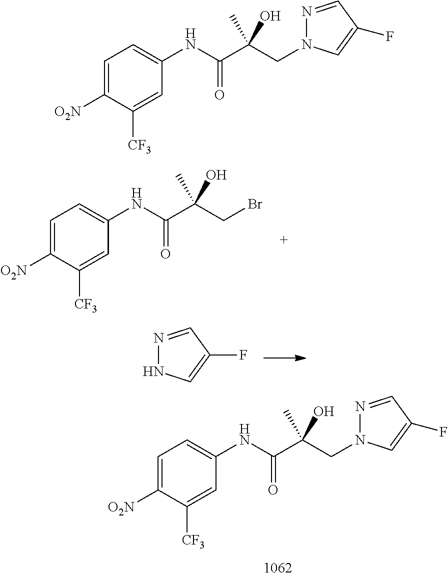 [SMILES: C[C@](O)(Cn1cc(F)cn1)C(=O)Nc1ccc([N+](=O)[O-])c(C(F)(F)F)c1 ; C[C@](O)(CBr)C(=O)Nc1ccc([N+](=O)[O-])c(C(F)(F)F)c1 ; Fc1cn[nH]c1 ; C[C@](O)(Cn1cc(F)cn1)C(=O)Nc1ccc([N+](=O)[O-])c(C(F)(F)F)c1]

To a dry, nitrogen-purged 100 mL round-bottom flask equipped with a dropping funnel under argon atmosphere, NaH of 60% dispersion in mineral oil (674 mg, 16.9 mmol) was added in 60 mL of anhydrous THF solvent in the flask at ice-water bath, and 4-fluoro-1H-pyrazole (691 mg, 8.03 mmol) was stirred in over 30 min at the ice-water bath. Into the flask, the solution of (R)-3-bromo-2-hydroxy-2-methyl-N-(4-nitro-3-(trifluoromethyl)phenyl)propanamide (2.98 g, 8.03 mmol) in 10 mL of anhydrous THF was added through dropping funnel under argon atmosphere at the ice-water bath and stirred overnight at room temperature. After adding 1 mL of H₂O, the reaction mixture was condensed under reduced pressure, and then dispersed into 50 mL of EtOAc, washed with 50 mL (×2) water, evaporated, dried over anhydrous MgSO₄, and evaporated to dryness. The mixture was purified with flash column chromatography as an eluent EtOAc/hexane=1/2 to produce designed compound (2.01 g, 67%) as yellowish solid.

MS (ESI) m/z 375.08 [M−H]⁻; 377.22 [M+H]⁺; 399.04 [M+Na]⁺;

¹⁹F NMR (CDCl₃, decoupled) δ −60.13, −176.47; assigned by NOE and COSY; ¹H NMR (400 MHz, CDCl₃) δ 9.14 (bs, 1H, NH), 8.01 (s, 1H), 7.97-7.91 (m, 2H), 7.38 (d, J=3.6 Hz, 1H), 7.35 (d, J=4.4 Hz, 1H), 5.95 (s, 1H, OH), 4.56 (d, J=14.0 Hz, 1H), 4.17 (d, J=14.0 Hz, 1H), 1.48 (s, 3H).

### (S)-3-(4-Fluoro-1H-pyrazol-1-yl)-2-hydroxy-2-methylpropanoic Acid (1062a)

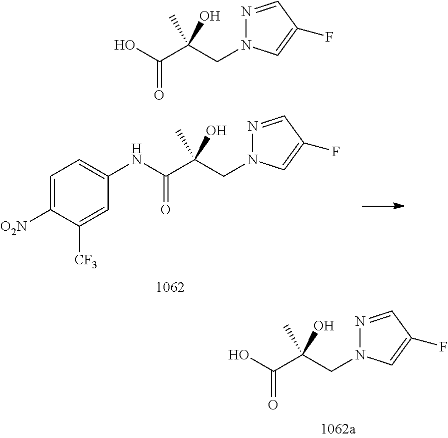

To a solution of 1062 (1.886 g, 5.29 mmol) in EtOH (40 ml) and water (20 ml) was added NaOH (424 mg, 10.59 mmol) and the reaction mixture was heated to reflux for 2 h, evaporated (to remove the EtOH) and then extracted with EtOAc. The aqueous phase was acidified to pH 1 and extracted with EtOAc. The extract was dried over Na₂SO₄, filtered and evaporated to afford the title compound (845 mg, 85%) as a brown oil. MS (ESI) m/z 187.06 [M−H]⁻; 188.91 [M+H]⁻;

¹⁹F NMR (acetone-d₆, decoupled) 6-0.24; assigned by NOE and COSY.

¹H NMR (400 MHz, acetone-d₆) δ 7.66 (d, J=4.4 Hz, 1H), 7.36 (d, J=4.0 Hz, 1H), 4.45 (d, J=14.0 Hz, 1H), 4.27 (d, J=14.0 Hz, 1H), 1.38 (s, 3H). ¹³C NMR (100 MHz, acetone-d₆) δ 175.70, 150.36 (d, J=24.12 Hz), 126.53 (d, J=13.6 Hz), 118.21 (d, J=28.0 Hz), 74.86, 60.59, 23.77.

### Preparation of (S)-N-(6-Cyano-5-(trifluoromethyl)pyridin-3-yl)-3-(4-(4-fluorophenyl)-1H-1,2,3-triazol-1-yl)-2-hydroxy-2-methylpropanamide (1063)

### (S)-3-Azido-N-(6-cyano-5-(trifluoromethyl)pyridin-3-yl)-2-hydroxy-2-methylpropanamide (1064)

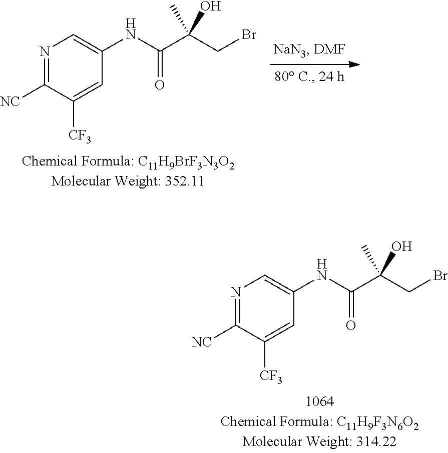 [SMILES: C[C@](O)(CBr)C(=O)Nc1cnc(C#N)c(C(F)(F)F)c1 ; C[C@](O)(CBr)C(=O)Nc1cnc(C#N)c(C(F)(F)F)c1]

A solution of (R)-3-bromo-N-(6-cyano-5-(trifluoromethyl)pyridin-3-yl)-2-hydroxy-2-methylpropanamide (352 mg, 1 mmol) in 10 mL of DMF was treated with NaN₃ (325 mg, 5 mmol) under Ar at 80° C. for 24 h. The reaction mixture was then cooled and extracted with CH₂Cl₂ (3×20 mL). The combined organic layers were washed with H₂O (3×20 mL) and brine, dried and evaporated to give a crude oil, which purified by silica gel chromatography (EtOAc/n-hexane=1:2, v/v) to afford product. Yield=87%;

MS (ESI) m/z 313.03 [M−H]⁻; ¹⁹F NMR (CDCl₃, decoupled) δ −62.11;

¹H NMR (400 MHz, CDCl₃) δ 9.16 (bs, 1H, NH), 8.89 (s, 1H), 8.77 (s, 1H), 3.90 (d, J=12.0 Hz, 1H), 3.52 (d, J=12.0 Hz, 1H), 3.20 (bs, 1H, OH), 1.55 (s, 3H).

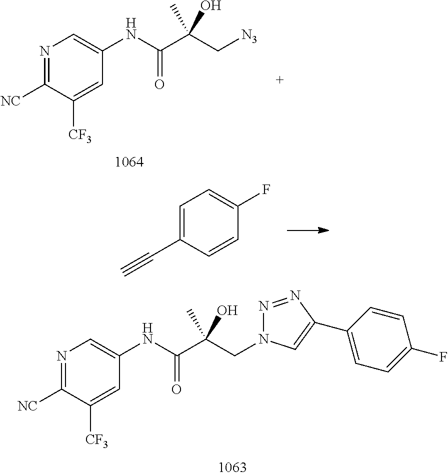

### (S)-N-(6-Cyano-5-(trifluoromethyl)pyridin-3-yl)-3-(4-(4-fluorophenyl)-1H-1,2,3-triazol-1-yl)-2-hydroxy-2-methylpropanamide (1063)

To a suspension of copper(I)iodide (11 mg, 0.055 mmoL) in acetonitrile (7 mL)/water (3 mL) mixture was added 1064 (57 mg, 0.182 mmol) at room temperature and then 1-ethynyl-4-fluorobenzene (0.015 mL, 0.182 mmol) was added. The resulting reaction mixture was stirred at room temperature for 3 days. The mixture was evaporated under reduced pressure, poured into water:brine (1:1) and then extracted with ethyl acetate. The combined organic extracts were then washed with brine, dried over sodium sulphate, filtered and evaporated. Purification by chromatography (silica, 60% ethyl acetate in hexane) to afford the product as a yellow solid (51.3 mg, 65%).

MS (ESI) m/z 433.09 [M−H]⁻ 435.06 [M+H]⁺;

¹⁹F NMR (acetone-d₆, decoupled) 6114.58, 61.66; assigned by NOE and COSY;

¹H NMR (400 MHz, acetone-d₆) δ 10.16 (bs, 1H, NH), 9.28 (s, 1H), 8.88 (s, 1H), 8.31 (s, 1H), 7.90 (t, J=7.8 Hz, 2H), 7.20 (t, J=8.8 Hz, 2H), 5.73 (bs, 1H, OH), 4.94 (d, J=14.2 Hz, 1H), 4.73 (d, J=14.2 Hz, 1H), 1.62 (s, 3H).

### Example 7

### In Vitro Assays to Determine SARD Activity

LNCaP or AD1 androgen receptor degradation (full length AR): The compounds of the invention were tested for their effect on full length AR protein expression. Methods: LNCaP or AD1 cells expressing full length AR were plated at 750,000-1,000,000 cells/well of a 6 well plate in growth medium (RPMI+10% FBS). Twenty four hours after plating, the medium was changed to RPMI+1% csFBS without phenol red and maintained in this medium for 2 days. The medium again was changed to RPMI+1% csFBS without phenol red and cells were treated with SARDs (1 nM to 10 M) in combination with 0.1 nM R1881. After 24 h of treatment, cells were washed with cold PBS and harvested. Protein was extracted using salt-containing lysis buffer with three freeze-thaw cycles. The protein concentration was estimated and five microgram of total protein was loaded on a SDS-PAGE, fractionated, and transferred to a PVDF membrane. The membrane was probed with AR N-20 antibody (SantaCruz Biotechnology, Inc., Dallas, Tex. 75220) and actin antibody (Sigma-Aldrich, St. Louis, Mo.) or GAPDH antibody (Sigma-Aldrich, St. Louis, Mo.).

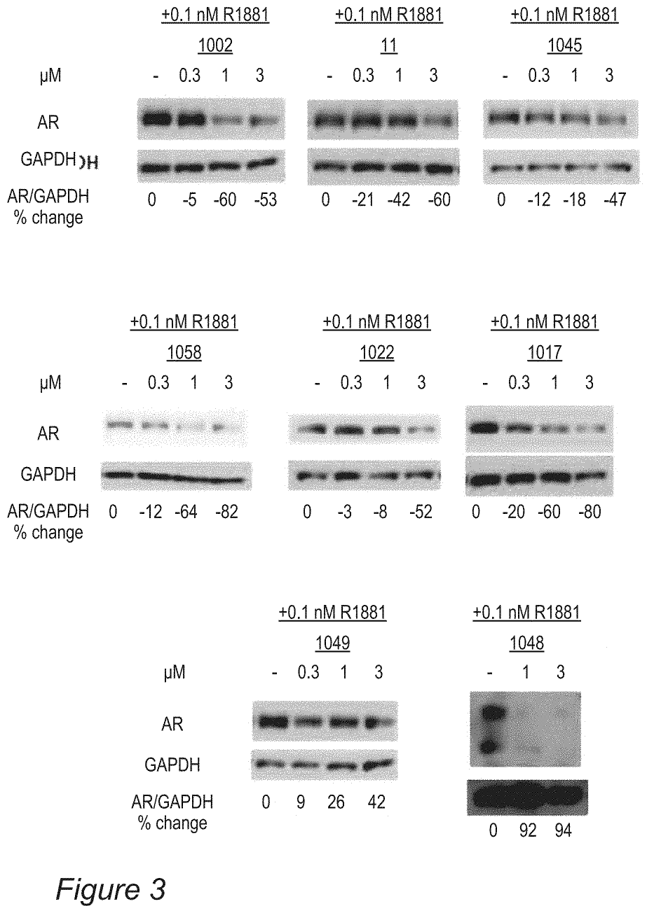

Results: Degradation in LNCaP or AD1 cells are reported in Table 1 in the column labeled ‘Full Length % Inhibition at 1, 10 μM’. The results of this assay are demonstrated in FIG. 3 as images of Western blot films (chemiluminescence exposed films). The numbers under each lane represents the % change from vehicle. The bands were quantified using ImageJ software. For each lane, the AR band was divided by GAPDH band and the % difference from vehicle was calculated and represented under each lane The numbers shown are 0 (no degradation) or represented as decreases in AR levels normalized for GAPDH levels (some values represented as positive but still indicate degradation). From this experiment, it is apparent that 1048, 1058 and 1017 are high efficacy AR degraders at a 3 M dose. Elsewhere herein 1048 is demonstrated to be intolerably toxic.

### Example 8

### In Vivo Antagonism of SARD Compounds

Hershberger method: Male mice (20-25 grams body weight; n=5-7/group) were either left intact or castrated (positive control) as indicated in the figures for 13 days. Intact rats were treated with the indicated compounds at the indicated dose by mouth daily for 13 days. Rats were sacrificed on day 14 of treatment and prostate and seminal vesicles organs were removed and weighed. Organ weights were either represented as is or were normalized to body weight. In overview, there was a 20 mg/kg fixed dose screening Hershberger in rats which was (performed in 2 batches) for compounds 11 (indole), 1002, 1002(Tart), 1017, 1022, 1045, 1048 (toxic so no data), 1049, 1058, 1065, and 1066; and subsequently a dose response (1, 5, 10, and 20 mg/kg) Hershberger experiment in rats performed with 1048 and 1065. The goal of the experiments was to find compounds with in vivo antiandrogen efficacies comparable or greater than 1002. Serum concentrations were determined for both experiments as reported in Tables 2 and 4.

Determining SARD compound serum concentrations in Hershberger rats (e.g., 1065 and 1048). Serum was collected 24-30 hours after the last dose. 100 μL of serum was mixed with 200 μL of acetonitrile/internal standard. Standard curves were prepared by serial dilution of standards in nM with 100 μL of rat serum, concentrations in nM were 1000, 500, 250, 125, 62.5, 31.2, 15.6, 7.8, 3.9, 1.9, 0.97, and 0. Standards were with extracted with 200 μL of acetonitrile/internal standard. The internal standard for this experiment was (S)-3-(4-cyanophenoxy)-N-(3-(chloro)-4-cyanophenyl)-2-hydroxy-2-methylpropanamide.

LC-MS/MS analysis: The analysis of the 1065 was performed using LC-MS/MS system consisting of Shimadzu Nexera X2 HPLC with an AB/Sciex Triple Quad 4500 Q-Trap™ mass spectrometer. The separation was achieved using a C₁₈ analytical column (Alltima™, 2.1×100 mm, 3 μm) protected by a C₁₈ guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 m/min at 70% A and 30% B. The total runtime for 1065 was 2.50 min, and the volume injected was 10 L. Multiple reaction monitoring (MRM) scans were made with curtain gas at 10; collision gas at medium; nebulizer gas at 60.0 and auxiliary gas at 60.0 and source temperature at 550° C. Molecular ions were formed using an ion spray voltage (IS) of 4200 (negative mode). Declustering potential (DP), entrance potential (EP), collision energy (CE), product ion mass, and cell exit potential (CXP) were optimized with the values of −75, −10, −30, and −13, respectively, for the mass pair 363.1/185.6. Serum concentrations of 1065 in individual rats dosed at 1, 5, 10, and 20 mg/kg are given below:

| | Extraction of 1065 | |
|---|---|---|
| Dose | Sample | Conc of 1065 |
| 1 mg/kg | Serum 1 | 81.57583 |
| | Serum 2 | 87.24625 |
| | Serum 3 | 120.1244 |
| | Serum 4 | 134.5505 |
| | Serum 5 | 129.9323 |
| 5 mg/kg | Serum 1 | 967.3936 |
| | Serum 2 | 798.7984 |
| | Serum 3 | 630.4879 |
| | Serum 4 | 691.1901 |
| | Serum 5 | 485.4264 |
| 10 mg/kg | Serum 1 | 637.641 |
| | Serum 2 | 915.1312 |
| | Serum 3 | 824.8076 |
| | Serum 4 | 681.9936 |
| | Serum 5 | 795.917 |
| 20 mg/kg | Serum 1 | 1286.902 |
| | Serum 2 | 1298.179 |
| | Serum 3 | 1360.687 |
| | Serum 4 | 1397.834 |
| | Serum 5 | 1193.986 |

Serum concentration determination for 1048. Serum was collected 24-30 hours after last dose. 100 μL of serum was mixed with 200 μL of acetonitrile/internal standard. Standard curves were prepared by serial dilution of standards in nM with 100 μL of rat serum, concentrations were 1000, 500, 250, 125, 62.5, 31.2, 15.6, 7.8, 3.9, 1.9, 0.97, and 0. Standards were with extracted with 200 μL of acetonitrile/internal standard. The internal standard for this experiment was (S)-3-(4-cyanophenoxy)-N-(3-(chloro)-4-cyanophenyl)-2-hydroxy-2-methylpropanamide.

LC-MS/MS analysis: The analysis of the 1048 was performed using LC-MS/MS system consisting of Shimadzu Nexera X2 HPLC with an AB/Sciex Triple Quad 4500 Q-Trap™ mass spectrometer. The separation was achieved using a C₁₈ analytical column (Alltima™, 2.1×100 mm, 3 μm) protected by a C₁₈ guard column (Phenomenex™ 4.6 mm ID cartridge with holder). Mobile phase was consisting of channel A (95% acetonitrile+5% water+0.1% formic acid) and channel C (95% water+5% acetonitrile+0.1% formic acid) and was delivered isocratically at a flow rate of 0.4 m/min at 70% A and 30% B. The total runtime for 1048 was 2.50 min, and the volume injected was 10 L. Multiple reaction monitoring (MRM) scans were made with curtain gas at 10; collision gas at medium; nebulizer gas at 60.0 and auxiliary gas at 60.0 and source temperature at 550° C. Molecular ions were formed using an ion spray voltage (IS) of 4200 (negative mode). Declustering potential (DP), entrance potential (EP), collision energy (CE), product ion mass, and cell exit potential (CXP) were optimized with the values of −100, −10, −34, and −9, respectively, for the mass pair 362.29/184.6.

| | Extraction of 1048 | |
|---|---|---|
| Dose | Sample | Conc of 1048 |
| 1 mg/kg | Serum 1 | 621.9929 |
| | Serum 2 | 760.1658 |
| | Serum 3 | 676.488 |
| | Serum 4 | 419.7344 |
| | Serum 5 | 370.0696 |
| 5 mg/kg | Serum 1 | 4191.451 |
| | Serum 2 | 1636.436 |
| | Serum 3 | 2047.831 |
| | Serum 4 | 3162.571 |

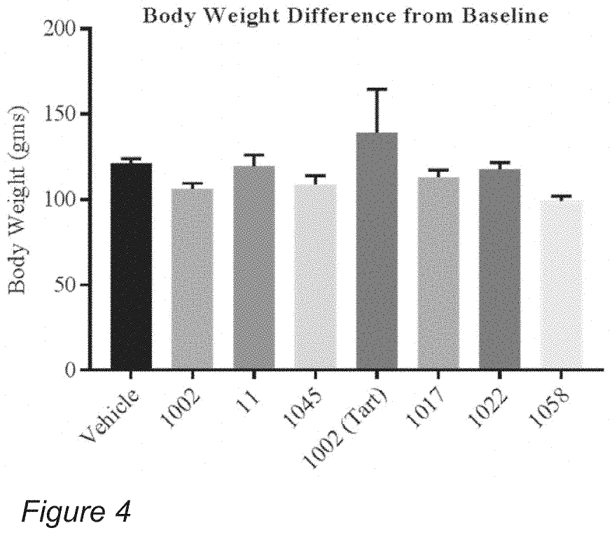

The first set of compounds that was tested at 20 mg/kg by mouth daily for 13 days are shown in FIG. 4. As can be seen, most of these compounds did not significantly decrease body weight, suggesting that there is no gross toxicity for these compounds at this dose.

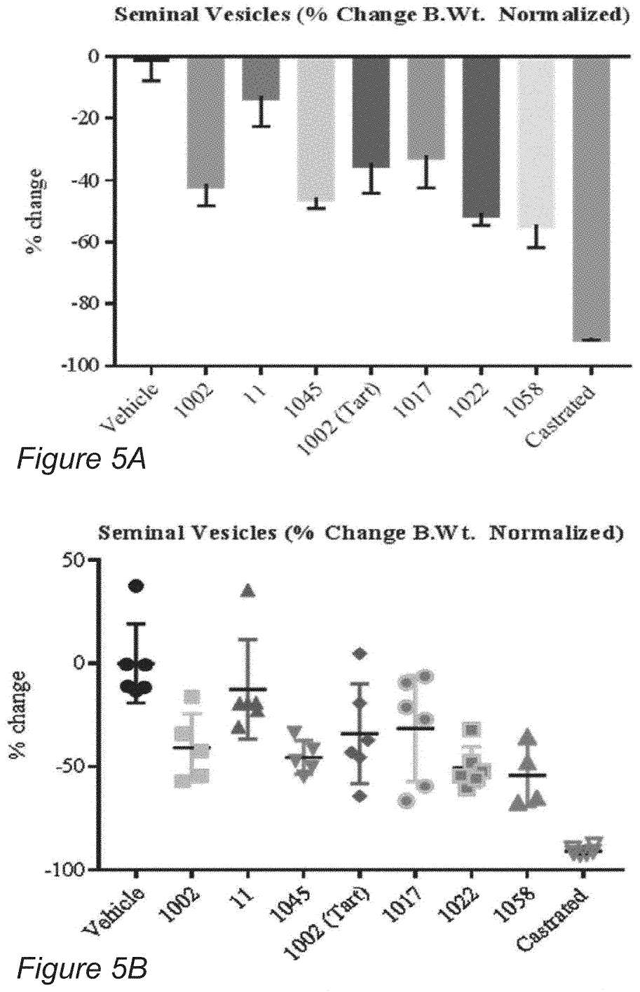

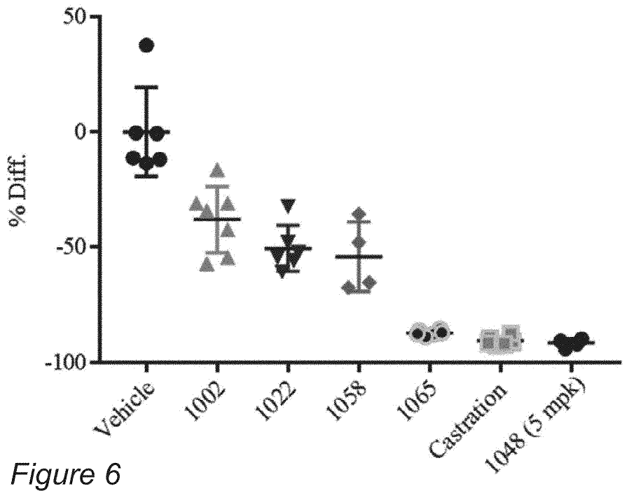

Table 2 (below) and FIGS. 5A-5B demonstrated AR antagonism in vivo for 1002, 11, 1045, 1002 (Tart), 1017, 1022, and 1058 as revealed in decreased support of the seminal vesicles organ weight, but none of these compounds attained chemical castration. Note that even though indole 11 is the most potent SARD in vitro (˜82 nM in Table 1), 11 was the weakest AR antagonist in vivo for this set of compounds, demonstrating less than 20% (13% inhibition of seminal vesicles weight in Table 2) changes from vehicle treated rats. Triazole 1045, pyrazoles 1017 and 1002 (Tart) performed equivocally to the pyrazole 1002 in this assay, whereas 1022 and 1058 exhibited greater efficacy at this dose. Correspondingly, 1022 and 1058 attained higher serum concentrations of 7-(64 nM) and 43 (404 nM)-fold greater than 1002 (9.3 nM) suggesting that the improved bioavailability of these SARDs translated into increased in vivo antiandrogenic efficacy. The improved bioavailability of 1058 (3-fluoro-4-bromopyrazole) vs. 1002 (4-fluoropyrazole) is unexpected in view of the minor structural differences. Interestingly, 1045 demonstrated 130-fold higher bioavailability (1209 nM) but only equivocal in vivo efficacy compared to 1002.

TABLE 2
Decrease in Seminal Vesicles Weights in Rats

| | | | | 1002 | | | |
|---|---|---|---|---|---|---|---|
| | 1002 | 1058 | 1022 | (Tart) | 11 | 1017 | 1045 |
| % inhibition | 41 | 54 | 51 | 34 | 13 | 32 | 46 |
| % difference | 0 | 32 | 24 | — | — | — | 12 |
| from 1002 | | | | | | | |
| P-value | 0.015 | 0.001 | 0.0006 | 0.048 | N.S. | N.S. | 0.005 |
| Serum | 9.3 | 404 | 64 | 12.4 | 0 | 0 | 1209 |
| conc. (nM) | | | | | | | |

The pattern of reductions in androgenic tissue weights was similar for prostate weights in this experiment. With respect to prostate weights 1058, 1022, 1017 and 1045 performed better than 1002. All reductions, relative to vehicle treated rats, (except 11) in prostate weight were significant (Table 3).

TABLE 3
Decrease in Prostate Weight in Rats

| | | | | 1002 | | | |
|---|---|---|---|---|---|---|---|
| | 1002 | 1058 | 1022 | (Tart) | 11 | 1017 | 1045 |
| % inhibition | 20 | 30 | 34 | 25 | 0 | 28 | 26 |
| % difference | 0 | 50 | 70 | | | 40 | 30 |
| from 1002 | | | | | | | |
| P Value | 0.049 | 0.021 | 0.0017 | 0.027 | N.S. | 0.0118 | 0.0018 |
| from vehicle | | | | | | | |

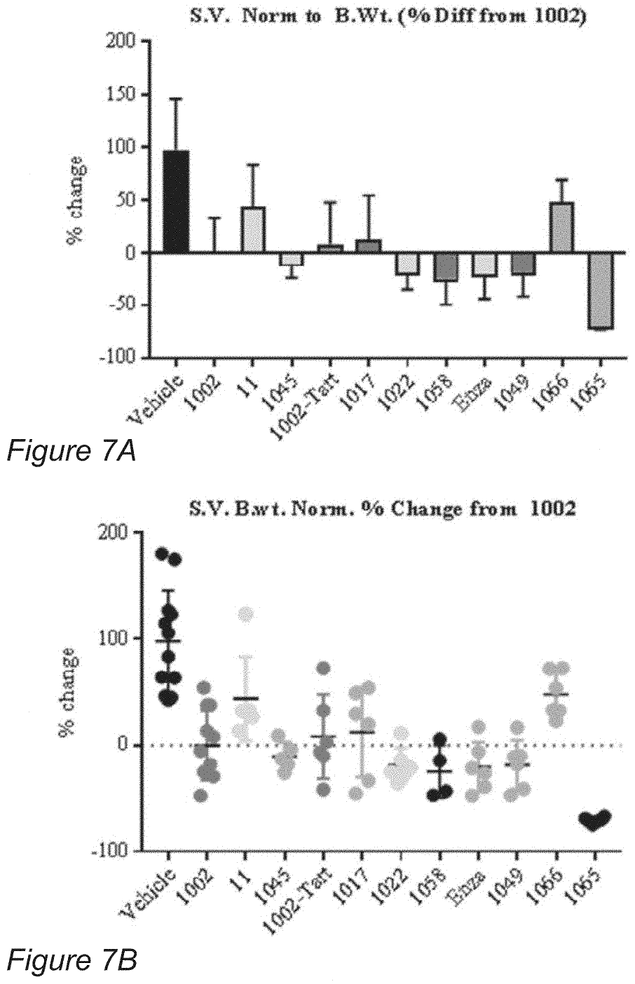

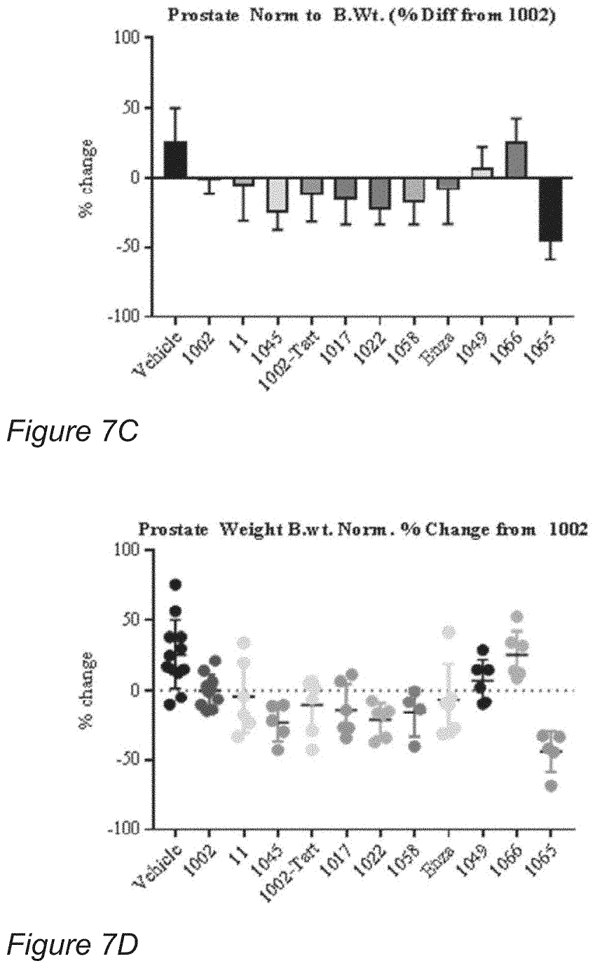

FIGS. 7A-7D present the % difference in organ weights from 1002 (% Diff from 1002) with 1002 defined as 0% change and vehicle defined as 100% change. When seminal vesicles (FIGS. 7A and 7B) weight reductions for all the compounds over the two studies are reported together, a few compounds (11 and 1066) were inferior to 1002, several compounds produced equivocal (1045, 1002(Tart), and 1017) to marginally improved (1022, 1058, enzalutamide (30 mg/kg), and 1049) efficacies compared to 1002, however, 1065 demonstrated significantly improved efficacy. Regarding prostate weight reductions (FIGS. 7C and 7D), most of the compounds demonstrated approximately 10-25% improved efficacies; however 1065 demonstrated approximately 45% improvement.

### Example 9

### Structure Activity Relationship

Table 4 reports the in vitro efficacy (transactivation/degradation) and metabolism (rat liver microsomes (RLM) and human LM (HLM) half-lives) data, in vivo efficacy (S.V. and prostate % diff in rats) and serum concentration in rats for all the compounds in the 20 mg/kg Hershberger experiment. Notably, triazole 1045 also demonstrated outstanding pharmacokinetic properties but relative weak AR antagonism. Similarly, 1049 improved bioavailability compared to 1002 by 4-fold but did not greatly increase in vivo antagonism at 20 mg/kg. Pyrazole 1058 (404 nM) demonstrated unexpected improved bioavailability compared to structural analogs 1022 (64 nM), 1002 (33 nM), 1049 (125 nM), and 1017 (0 nM). In overview, 1058 and 1065 have a combination of excellent in vitro properties, in vivo efficacy, and pharmacokinetic properties that make these molecules unique.

TABLE 4
In Vitro and In Vivo Antagonism (rats), and Serum Concentrations (rats)

| | 1048 | Castration | [symbol] | 1058 | 1022 | 1022 | 1045 | 1049 | 11 | 1017 | [symbol] |
|---|---|---|---|---|---|---|---|---|---|---|---|
| Transactivation (nM) | 40.8 | | 59.4 | 83.7 | [symbol] | 219.5 | 183.3 | [symbol] | 28.99 | 71.2 | [symbol] |
| Degradation | 87 | | 89 | 82 | [symbol] | [symbol] | 47 | 42 | [symbol] | 80 | 0 |
| (% at 3 μm) | | | | | | | | | | | |
| RLM | 86 | | 84 | 400 | [symbol] | 78 | 400 | 179 | 11 | 400 | 300 |
| Metabolism | | | | | | | | | | | |
| (H.L. min) | | | | | | | | | | | |
| HLM | 400 | | STABLE 10 % | 138 | [symbol] | 82 | 204 | [symbol] | 6 | 400 | 319 |
| Metabolism | | | metabolized | | | | | | | | |
| (H.L. min) | | | after 60 min) | | | | | | | | |
| (S.V.) % diff | .90 | .90 | .88 | .54 | .50 | .40 | .45 | .65 | .12 | .31 | .35 |
| | [symbol] | | | | | | | | | | |
| (Prostate) | .90 | .88 | .58 | .30 | .34 | .24 | .35 | .20 | .19 | .28 | .9 |
| % diff | [symbol] | | | | | | | | | | |
| Serum Conc. (nM) | | | 561 | 404 | 64 | 33 | 1209 | 125 | 0 | 0 | 19 |

[symbol]

indicates data missing or illegible when filed

### Example 10

### Chemical Castration without Lowering Serum Testosterone

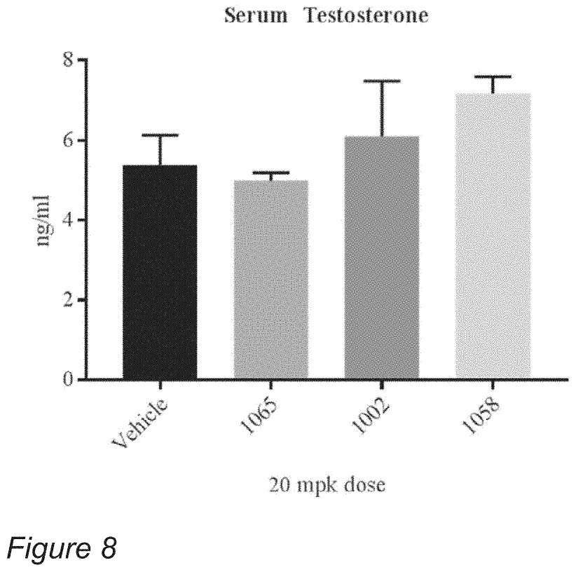

For the rats treated with the 20 mg/kg, blood was drawn at the time of sacrifice and serum isolated. The serum was run through a LC-MS/MS to detect testosterone levels. As can be seen in FIG. 8, even at levels much higher than those that produced chemical castration with 1065, there is no significant reduction in serum testosterone levels. Similar results were obtained for 1002 and 1058. This indicated that SARDs do not have any effect of the synthesis of testosterone but were potent in vivo AR antagonists by virtue of direct effects on AR. Further, this highlights that SARDs are potent antagonists which are capable of overcoming the endogenous androgens present in intact animals.

### Example 11

### AR and GR Co-antagonists

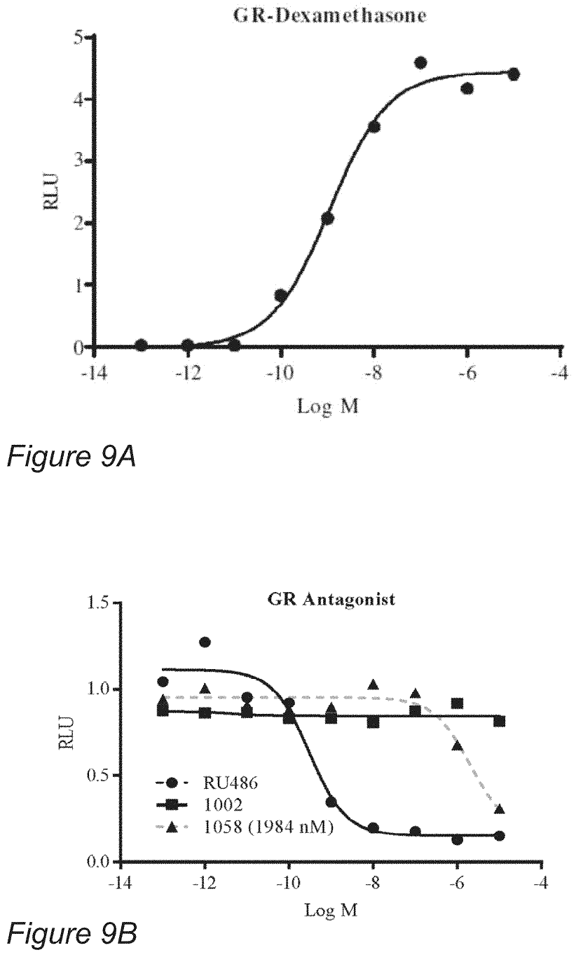

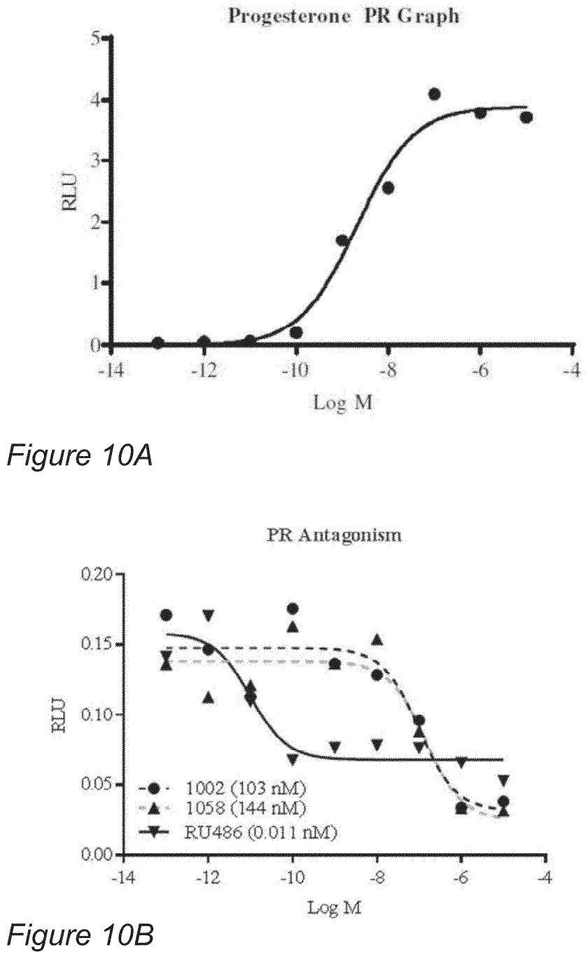

1058 is a potent AR antagonist in vitro (83.7 nM) and capable of SV and FL AR degradation (70 and 80%, respectively). A transcriptional activation assay using dexamethasone of as positive control (FIG. 9A) confirmed that GR agonism as dexamethasone demonstrated potent and full efficacy agonism in this assay system. Further, a dose response of 1058 in this transcriptional activation assay in antagonist mode produced GR antagonism (IC₅₀ of 1984 nM) which was less potent but complete like RU486 in vitro (FIG. 9B). 1002 (FIG. 9B) and other SARDs of this template (not shown) produced no antagonism in this assay at concentrations up to 10 M. The GR antagonism for 1058 is unexpected in view of the slight structural difference between 1002 and 1058. Further, in view of the unexpected bioavailability of 1058 relative to 1002 and other pyrazoles reported herein (see Example 10) suggests that 1058 may be able to overcome or prevent the emergence of antiandrogen resistance mediated by GR, as discussed in Horm Cancer. (2014) 5(2), 72-89 or doi:10.1007/s12672-014-0173-2 and Cell (2013) 155, 1309-1322 or doi: 10.1016/j.cell.2013.11.012. 1058 and most of the SARDs as disclosed herein (1002 is shown) are also a potent PR antagonists in vitro (FIG. 10B) suggesting the possibility of the treatment of breast cancers as well. The endogenous progestin, progesterone, was used as a positive control and produced potent and full efficacy agonist activity in this system. (FIG. 10A).

### Example 12

### Unexpected Antagonism of AR-V7 Dependent Activity with Compound 1058

### LNCaP-ARV7 Degradation Assay

Method: LNCaP-ARV7 cells expressing full length AR and inducibly expressing AR-V7 as described in the literature (PMID's: 26378018 and 25008967) were plated at 750,000-1,000,000 cells/well of a 6 well plate in growth medium (RPMI+10% FBS). Twenty four hours after plating, medium was changed and treated with 10 ng/mL doxycycline and SARDs as indicted. After 24 h of treatment, cells were washed with cold PBS and harvested. Protein was extracted using salt-containing lysis buffer with three freeze-thaw cycles. Protein concentration was estimated and five microgram of total protein was loaded on a SDS-PAGE, fractionated, and transferred to a PVDF membrane. The membrane was probed with AR N-20 antibody from SantaCruz and GAPDH antibody (Sigma-Aldrich, St. Louis, Mo.).

### 22RV1 Gene Expression Assay

Methods: Gene expression studies were performed in 22RV1 cells (which endogeneously express AR-V7) plated in 96 well plate at 10,000 cells/well in RPMI+1% csFBS. Cells were maintained in this medium for 3 days and then treated for 24 h and RNA was isolated using cells-to-ct reagent, cDNA synthesized, and expression of various genes was measured by realtime rtPCR (ABI 7900) using Taqman primers and probes. Gene expression results were normalized to GAPDH.

### LNCaP-ARV7 Proliferation in Full Serum Assay

LNCaP-ARV7 Cell Growth Assay: Cells were plated at 10,000 cells/well in RPMI+10% FBS in 96 well plates. Cells were treated in the indicated medium with a dose response of the SARDs in the presence of doxycycline. At the end of three days, medium was changed and the cells were re-treated. At the end of 6 days, the live cells were measured by Cell-Titer-Glo (Promega) assay.

### LNCaP-ARV7 Gene Expression Assay

Method: LNCaP-ARV7 cells were plated at 15,000 cells/well of a 96 well plate in RPMI+1% csFBS without phenol red. Forty-eight hours after plating, cells were treated with a dose response of SARDs in the presence of 10 ng/mL of doxycycline in the presence of R1881. Twenty four hours after treatment, RNA was isolated using cells-to-ct reagent, cDNA synthesized, and expression of various genes was measured by realtime rtPCR (ABI 7900) using Taqman primers and probes. Gene expression results were normalized to GAPDH.

### Results

The androgen receptor supports the growth of prostate cancer cells, and in response to treatment with LBD-directed antagonists such as all of the FDA approved anti-androgen and CYP 17 inhibitor therapies, prostate cancer cells express various androgen receptor splice variants (AR-SV) lacking the LBD and allowing for constitutive and ligand-independent activation of the AR axis as discussed in the literature (PMID's: 26378018, 25008967, and many others). The constitutive activity of AR-SVs including the androgen receptor (AR) splice variant 7 (AR-V7) allows tumor growth despite castration levels of androgen which is termed as castration-resistant prostate cancer (CRPC). AR-V7, or alternatively abbreviated as ARV7, is one such AR-SV that mediates resistance to LBD-directed antagonists such as enzalutamide and abiraterone. In an effort to demonstrate that the SARDs of the invention can antagonize and degrade AR-V7 and overcome AR-V7 dependent CRPC, models of prostate cancer which expresses AR-V7 were studied with regard to the ability to induce AR-V7 degradation (LNCaP-ARV7 degradation assay described above), and antagonize AR-V7 dependent gene expression (22RV1 gene expression and LNCaP-ARV7 gene expression assays), and cellular proliferation (LNCaP-ARV7 proliferation in full serum assay). Unexpectedly 1058 demonstrated superior results compared to 1002 in all four assays. 1002 is a structural analog and a previous lead SARD.

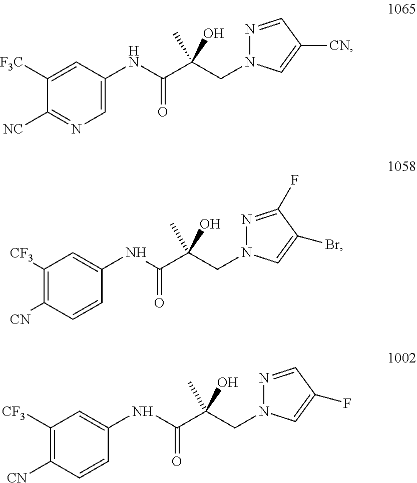

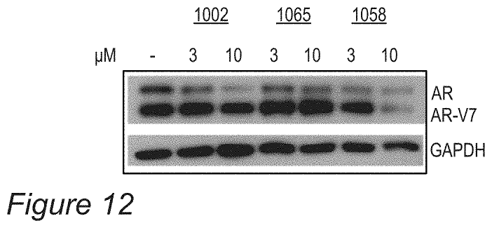

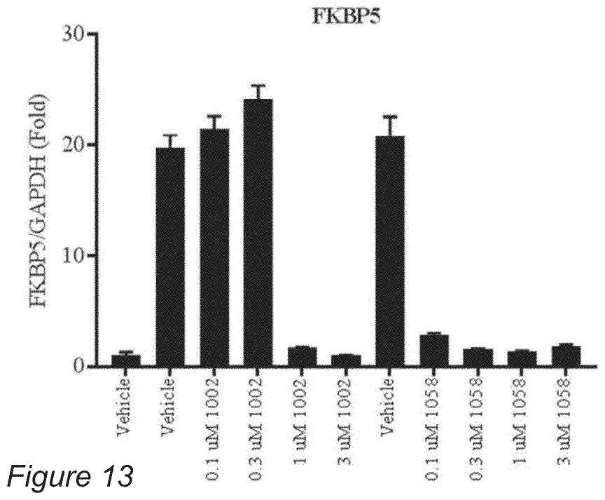

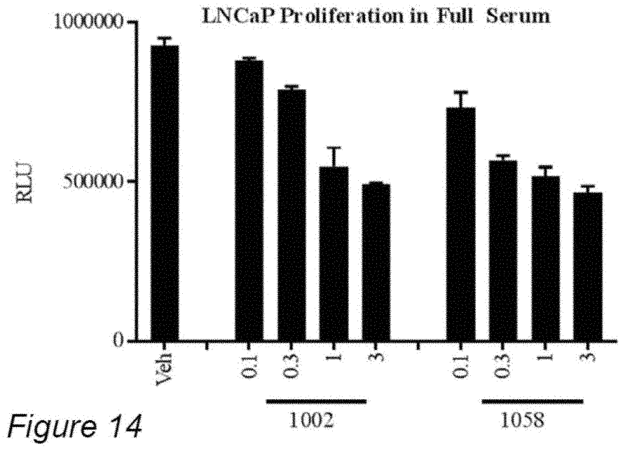

LNCaP-ARV7 prostate cancer cells endogeneously and constitutively express a full length AR (AR) and possess a stably incorporated tetracycline-induced AR-V7 expression construct. Addition of doxycycline induces the expression of AR-V7 in these cells and serves as a model of AR-V7 dependent prostate cancers. FIG. 12 shows that while the previous lead molecule 1002 (4-fluoropyrazole) was able to reduce AR levels but not AR-V7 levels at 3 and 10 M, 1058 (3-fluoro-4-bromopyrazole) almost completely eliminated both AR and AR-V7 at 10 M in LNCaP-ARV7 cells expressing AR-V7. (1065 was also tested in this experiment.) FIG. 13 demonstrated an unexpected 10-fold increased potency of 1058 in the antagonism of R1881 induced expression of FKBP5, a classically known AR-dependent gene, in LNCaP-ARV7 cells. Similarly, FIG. 14 shows that 1058 more potently inhibited AR-V7 dependent proliferation of LNCaP-ARV7 cells with antagonism seen of 0.3 M for 1058 vs. 1 M for 1002. The increased potency and efficacy of 1058 vs. 1002 to degrade and inhibit the activity of AR-V7 in prostate cancer cells was unexpected and suggested improved ability to treat AR-SV dependent prostate cancers including presently untreatable CRPCs.

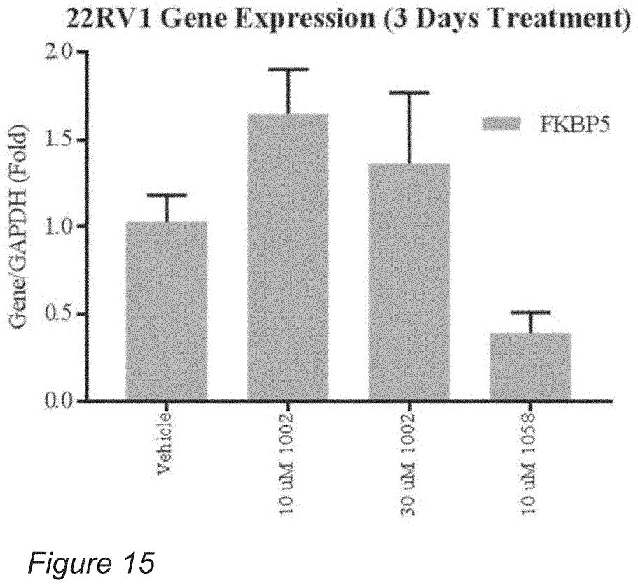

The above model of CRPC possessed an inducible AR-V7, whereas 22RV1 prostate cancer cells endogeneously and constitutively express both full length AR (AR) and AR-V7. The bulk of the baseline AR-axis activity (i.e., in the absence of an added androgen) in 22RV1 cells is believed to be due to AR-V7 activity as reflected by poor AR antagonism of proliferation and AR-dependent genes by LBD-directed anti-androgens such as enzalutamide in 22RV1 (not shown). FIG. 15 shows that 1058 but not 1002 was able to inhibit the AR-V7 dependent baseline expression (i.e., in the absence of an added androgen to active AR-FL) of the AR-dependent gene FKBP5 in 22RV1 cells. Given the structural similarity of 1058 and 1002, it is unexpected that 1058 was >3-fold more potent than 1002. Further, 1002 did not demonstrate any efficacy in the dose range tested where as 1058 inhibition was in excess of 50% at 10 M.

Cumulatively, these results suggest that 1058 will be able to achieve unexpected efficacy in CRPCs expressing AR-SVs compared to 1002 and other previous SARDs.

### Example 13

### Synthesis of Compound 1068

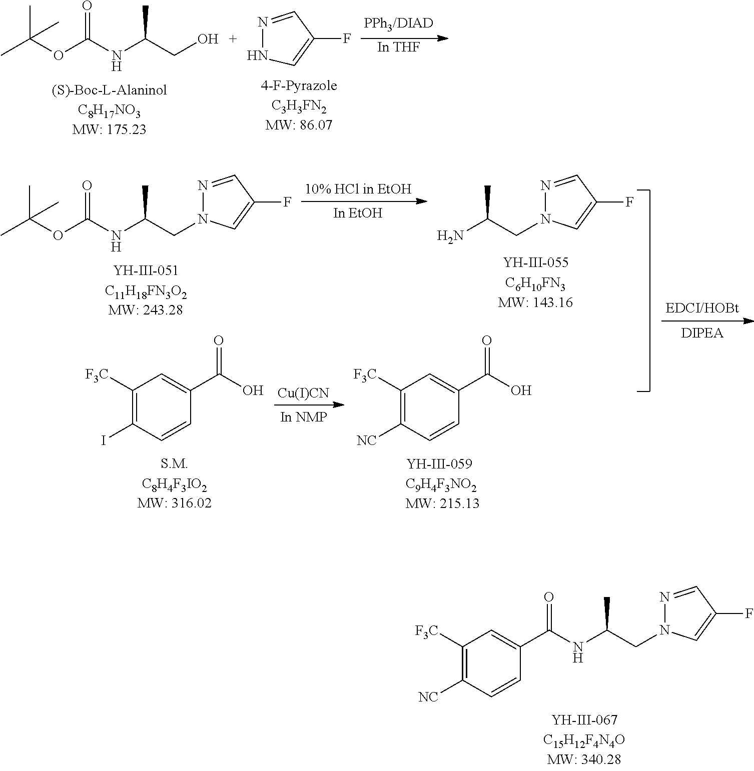

### (S)-Tert-butyl (1-(4-fluoro-1H-pyrazol-1-yl)propan-2-yl)carbamate (C₁₁H₁₈FN₃O₂)

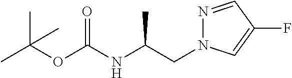 [SMILES: C[C@@H](Cn1cc(F)cn1)NC(=O)OC(C)(C)C]

A mixture of (S)-tert-butyl (1-hydroxypropan-2-yl)carbamate (3.05 g, 0.0174277 mol), 4-fluoro-1H-pyrazole (1.00 g, 0.01161845 mol), PPh₃ (4.57 g, 0.0174277 mol), DIAD (4.01 g, 0.0174277 mol), and 10 mL of ethyl acetate was allowed to stir at 20° C. for overnight under an argon atmosphere. The reaction was quenched by water, and extracted with ethyl acetate. The organic layer was washed with brine, dried with MgSO₄, filtered, and concentrated under vacuum. After work-up, the crude product was used in the next step reaction without further purification.

Mass (ESI, Negative): 242.21 [M−H]⁻;

### (S)-1-(4-Fluoro-1H-pyrazol-1-yl)propan-2-amine (C₆H₁₀FN₃)

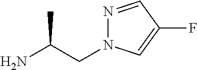

(S)-Tert-butyl (1-(4-fluoro-1H-pyrazol-1-yl)propan-2-yl)carbamate (2.78 g, 0.0174277 mol) was dissolved in 80 mL of 10% HCl in ethanol, and the resulting reaction mixture was allowed to stir for overnight under an argon atmosphere. After work-up, the crude product (0.38 g, 23.3% yield in two steps) as yellow oil was used in the next step reaction without further purification.

Mass (ESI, Positive): 144.02 [M+H]⁺.

### 4-Cyano-3-(trifluoromethyl)benzoic Acid (C₉H₄F₃NO₂)

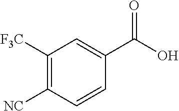 [SMILES: N#Cc1ccc(C(=O)O)cc1C(F)(F)F]

A mixture of 4-iodo-3-(trifluoromethyl)benzoic acid (2.00 g, 0.0063287 mol), Cu(I)CN (1.70 g, 0.018986 mol), PPh₃ (2.49 g, 0.009493 mol), and 40 mL of dry DMF was heated at 100±5° C. for 14 h. After the end of the reaction was established by TLC, the reaction mixture was partitioned in ethyl acetate and water, and extracted the water layer with ethyl acetate. The combined organic layers were washed with brine, dried over MgSO₄, filtered, and concentrated under vacuum. Product was purified by a silica gel column using hexane and ethyl acetate (4:1 to 3:1 and then 2:1) as eluent to afford 1.10 g (85%) of the titled compound as light brown solid.

¹H NMR (400 MHz, DMSO-d₆) δ 14.02 (br s, 1H, OH), 8.39-8.32 (m, 2H, ArH), 7.41-7.31 (m, 1H, ArH).

Mass (ESI, Negative):213.91 [M−H]⁻;

### (S)-4-Cyano-N-(1-(4-fluoro-1H-pyrazol-1-yl)propan-2-yl)-3-(trifluoromethyl)benzamide (C₁₅H₁₂F₄N₄O) (Compound 1068)

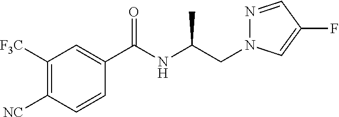 [SMILES: C[C@@H](Cn1cc(F)cn1)NC(=O)c1ccc(C#N)c(C(F)(F)F)c1]

A mixture of (S)-1-(4-fluoro-1H-pyrazol-1-yl)propan-2-amine (0.37 g, 0.0025845 mol), 4-cyano-3-(trifluoromethyl)benzoic acid (0.67 g, 0.0031014 mol), EDCI (0.60 g, 0.0038768 mol), HOBt (0.12 g, 0.000773 mol), DIPEA (0.67 g, 0.005196 mol), and 30 mL of dry DMF was allowed to stir for 3 days under an argon atmosphere. The reaction was quenched by water, and extracted with ethyl acetate. The organic layer was washed with brine, dried with MgSO₄, filtered, and concentrated under vacuum. The product was purified by a silica gel column using hexane and ethyl acetate (2:1 to 1:1) as eluent to afford 0.15 g (17%) of the titled compound as off-white solid.

¹H NMR (400 MHz, DMSO-d₆) δ 8.82 (d, J=8.0 Hz, 1H, NH), 8.33-8.29 (m, 2H, ArH), 8.24 (d, J=8.0 Hz, 1H, ArH), 7.87 (d, J=4.8 Hz, 1H, Pyrazole-H), 7.44 (d, J=4.0 Hz, 1H, Pyrazole-H), 4.43-4.36 (m, 1H, CH), 4.21-4.11 (m, 2H, 2×CH), 1.55 (d, J=6.8 Hz, 3H, CH₃).

Mass (ESI, Negative): 339.22 [M−H]⁻; (ESI, Positive): 341.17 [M+H]⁺.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A selective androgen receptor degrader (SARD) compound represented by any one of the following compounds: 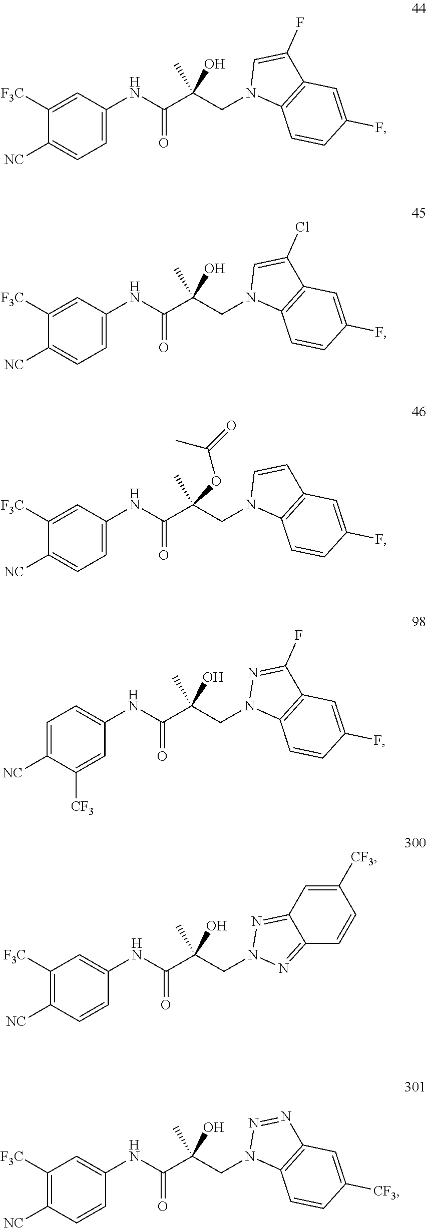 [SMILES: C[C@](O)(Cn1cc(F)c2cc(F)ccc21)C(=O)Nc1ccc(C#N)c(C(F)(F)F)c1 ; C[C@](O)(Cn1cc(Cl)c2cc(F)ccc21)C(=O)Nc1ccc(C#N)c(C(F)(F)F)c1 ; CC(=O)O[C@@](C)(Cn1ccc2cc(F)ccc21)C(=O)Nc1ccc(C#N)c(C(F)(F)F)c1 ; C[C@](O)(Cn1nc(F)c2cc(F)ccc21)C(=O)Nc1ccc(C#N)c(C(F)(F)F)c1 ; C[C@](O)(Cn1nc2ccc(C(F)(F)F)cc2n1)C(=O)Nc1ccc(C#N)c(C(F)(F)F)c1 ; C[C@](O)(Cn1nnc2cc(C(F)(F)F)ccc21)C(=O)Nc1ccc(C#N)c(C(F)(F)F)c1] 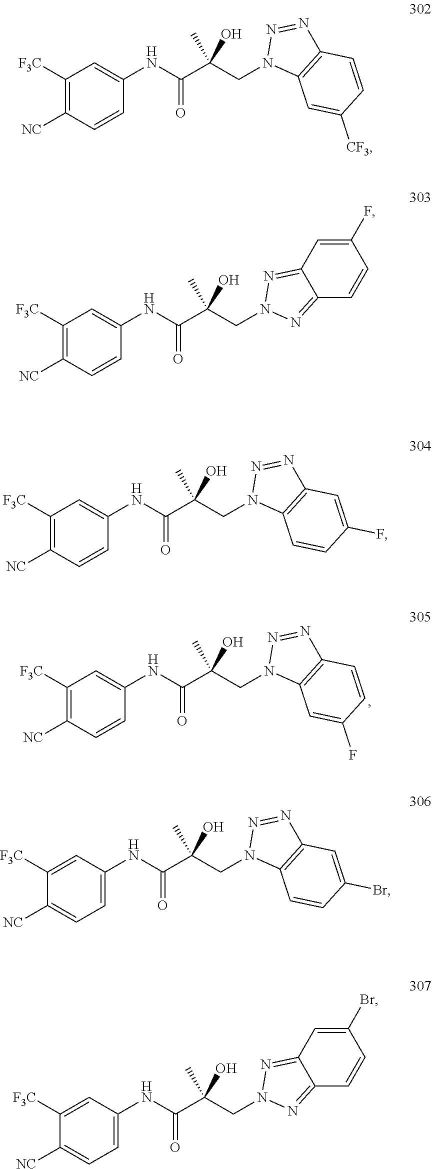 [SMILES: C[C@](O)(Cn1nnc2ccc(C(F)(F)F)cc21)C(=O)Nc1ccc(C#N)c(C(F)(F)F)c1 ; C[C@](O)(Cn1nc2ccc(F)cc2n1)C(=O)Nc1ccc(C#N)c(C(F)(F)F)c1 ; C[C@](O)(Cn1nnc2cc(F)ccc21)C(=O)Nc1ccc(C#N)c(C(F)(F)F)c1 ; C[C@](O)(Cn1nnc2ccc(F)cc21)C(=O)Nc1ccc(C#N)c(C(F)(F)F)c1 ; C[C@](O)(Cn1nnc2cc(Br)ccc21)C(=O)Nc1ccc(C#N)c(C(F)(F)F)c1 ; C[C@](O)(Cn1nc2ccc(Br)cc2n1)C(=O)Nc1ccc(C#N)c(C(F)(F)F)c1] 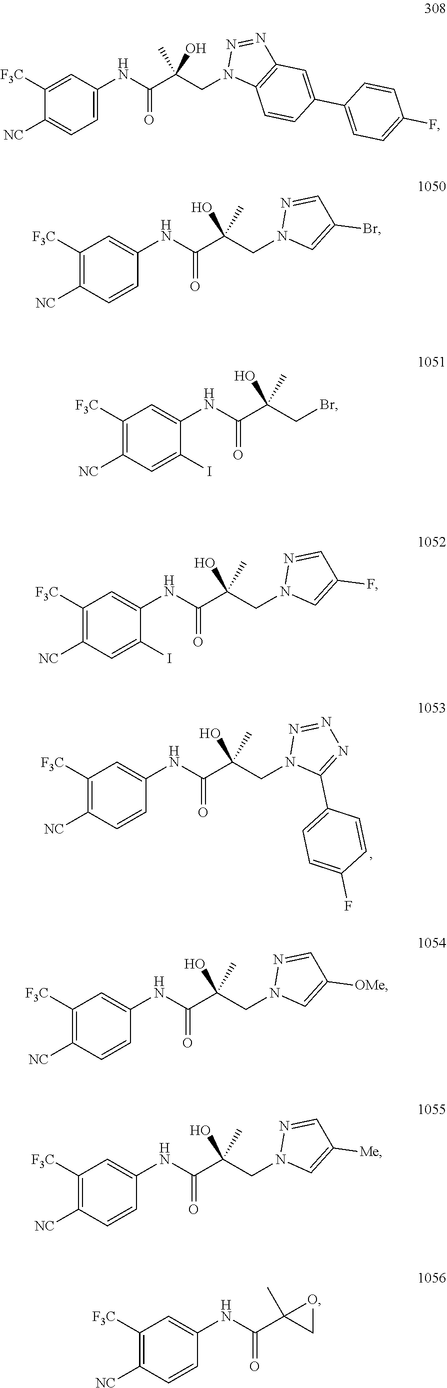 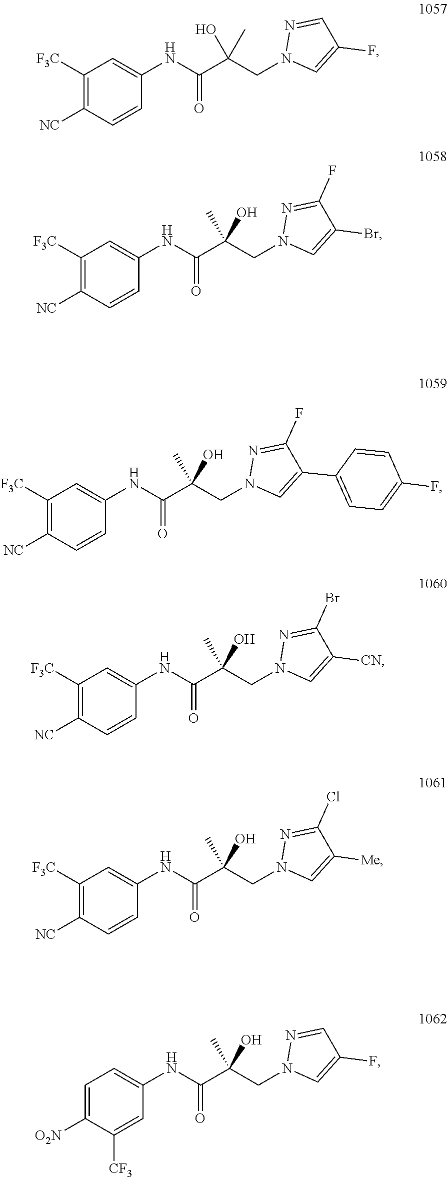 [SMILES: CC(O)(Cn1cc(F)cn1)C(=O)Nc1ccc(C#N)c(C(F)(F)F)c1 ; C[C@](O)(Cn1cc(Br)c(F)n1)C(=O)Nc1ccc(C#N)c(C(F)(F)F)c1 ; C[C@](O)(Cn1cc(-c2ccc(F)cc2)c(F)n1)C(=O)Nc1ccc(C#N)c(C(F)(F)F)c1 ; C[C@](O)(Cn1cc(C#N)c(Br)n1)C(=O)Nc1ccc(C#N)c(C(F)(F)F)c1 ; Cc1cn(C[C@](C)(O)C(=O)Nc2ccc(C#N)c(C(F)(F)F)c2)nc1Cl ; C[C@](O)(Cn1cc(F)cn1)C(=O)Nc1ccc([N+](=O)[O-])c(C(F)(F)F)c1] 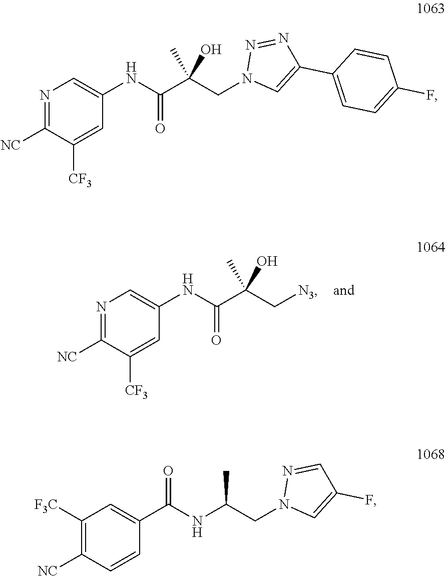 [SMILES: C[C@](O)(Cn1cc(-c2ccc(F)cc2)nn1)C(=O)Nc1cnc(C#N)c(C(F)(F)F)c1 ; C[C@](O)(CN=[N+]=[N-])C(=O)Nc1cnc(C#N)c(C(F)(F)F)c1 ; "and" ; C[C@@H](Cn1cc(F)cn1)NC(=O)c1ccc(C#N)c(C(F)(F)F)c1]
or its optical isomer, isomer, pharmaceutically acceptable salt, pharmaceutical product, polymorph, hydrate, or any combination thereof.

2. The compound according to claim 1, wherein the compound exhibits at least one of AR-splice variant (AR-SV) degradation activity, full length (AR-FL) degradation activity, AR-SV inhibitory, or AR-FL inhibitory activity.

3. A pharmaceutical composition comprising a SARD compound according to claim 1, or its isomer, optical isomer or mixture thereof including the racemic mixture, pharmaceutically acceptable salt, pharmaceutical product, hydrate or any combination thereof, and a pharmaceutically acceptable carrier.

4. The pharmaceutical composition according to claim 3, wherein the composition is formulated for topical use.

5. The pharmaceutical composition according to claim 4, wherein the composition is in the form of a solution, lotion, salve, cream, ointment, liposome, spray, gel, foam, roller stick, cleansing soap or bar, emulsion, mousse, aerosol, or shampoo.

6. A method of treating prostate cancer (PCa) or increasing the survival of a male subject suffering from prostate cancer comprising administering to the subject a therapeutically effective amount of a compound according to claim 1, or its isomer, pharmaceutically acceptable salt, pharmaceutical product, hydrate or any combination thereof.

7. The method according to claim 6, wherein the prostate cancer is at least one of advanced prostate cancer, refractory prostate cancer, castration resistant prostate cancer (CRPC), metastatic CRPC (mCRPC), non-metastatic CRPC (nmCRPC), or high-risk nmCRPC.

8. The method according to claim 6 further comprising administering androgen deprivation therapy (ADT).

9. The method according to claim 6, wherein the prostate cancer is resistant to treatment with an androgen receptor antagonist(s).

10. The method according to claim 9, wherein the androgen receptor antagonist is at least one of darolutamide, enzalutamide, apalutamide, bicalutamide, abiraterone, ODM-201 (darolutamide), EPI-001, EPI-506, AZD-3514, galeterone, ASC-J9, flutamide, hydroxyflutamide, nilutamide, cyproterone acetate, ketoconazole, or spironolactone.

11. A method of treating enzalutamide resistant prostate cancer in a subject comprising administering to the subject a therapeutically effective amount of a compound according to claim 1, or its isomer, pharmaceutically acceptable salt, pharmaceutical product, hydrate or any combination thereof.

12. A method of treating apalutamide resistant prostate cancer in a subject comprising administering to the subject a therapeutically effective amount of a compound according to claim 1, or its isomer, pharmaceutically acceptable salt, pharmaceutical product, hydrate or any combination thereof.

13. A method of treating abiraterone resistant prostate cancer comprising administering to the subject a therapeutically effective amount of a compound according to claim 1, or its isomer, pharmaceutically acceptable salt, pharmaceutical product, hydrate or any combination thereof.

14. A method of treating triple negative breast cancer in a subject comprising administering to the subject a therapeutically effective amount of a compound according to claim 1, or its isomer, pharmaceutically acceptable salt, pharmaceutical product, hydrate or any combination thereof.

15. A method of reducing the levels of AR-splice variants in a subject comprising administering to the subject a therapeutically effective amount of a compound according to claim 1, or its isomer, pharmaceutically acceptable salt, pharmaceutical product, hydrate or any combination thereof.

16. The method according to claim 15, wherein the method further reduces the levels of AR-full length (AR-FL) in the subject.

17. A method of treating Kennedy's disease in a subject comprising administering to the subject a therapeutically effective amount of the compound of claim 1, or its isomer, pharmaceutically acceptable salt, pharmaceutical product, hydrate or any combination thereof.

18. A method of treating acne in a subject comprising administering to the subject a therapeutically effective amount of a compound according to claim 1, or its isomer, pharmaceutically acceptable salt, pharmaceutical product, hydrate or any combination thereof.

19. The method according to claim 18, wherein the acne is acne vulgaris.

20. A method of decreasing sebum production in a subject comprising administering to the subject a therapeutically effective amount of a compound according to claim 1, or its isomer, pharmaceutically acceptable salt, pharmaceutical product, hydrate or any combination thereof.

21. The method according to claim 20, wherein decreasing sebum production treats at least one of seborrhea, seborrheic dermatitis, or acne.

22. A method of treating hirsutism or alopecia in a subject comprising administering to the subject a therapeutically effective amount of a compound according to claim 1, or its isomer, pharmaceutically acceptable salt, pharmaceutical product, hydrate or any combination thereof.

23. The method according to claim 22, wherein the alopecia is at least one of androgenic alopecia, alopecia areata, alopecia secondary to chemotherapy, alopecia secondary to radiation therapy, alopecia induced by scarring, or alopecia induced by stress.

24. A method of treating a hormonal condition in a female comprising administering to the female a therapeutically effective amount of a compound according to claim 1, or its isomer, pharmaceutically acceptable salt, pharmaceutical product, hydrate or any combination thereof.

25. The method according to claim 24, wherein the hormonal condition is at least one of precocious puberty, dysmenorrhea, amenorrhea, multilocular uterus syndrome, endometriosis, hysteromyoma, abnormal uterine bleeding, early menarche, fibrocystic breast disease, fibroids of the uterus, ovarian cysts, polycystic ovary syndrome, pre-eclampsia, eclampsia of pregnancy, preterm labor, premenstrual syndrome, or vaginal dryness.

26. A method of treating sexual perversion, hypersexuality, or paraphilias in a subject comprising administering to the subject a therapeutically effective amount of a compound according to claim 1, or its isomer, pharmaceutically acceptable salt, pharmaceutical product, hydrate or any combination thereof.

27. A method of treating androgen psychosis in a subject comprising administering to the subject a therapeutically effective amount of a compound according to claim 1, or its isomer, pharmaceutically acceptable salt, pharmaceutical product, hydrate or any combination thereof.

28. A method of treating virilization in a subject comprising administering to the subject a therapeutically effective amount of a compound according to claim 1, or its isomer, pharmaceutically acceptable salt, pharmaceutical product, hydrate or any combination thereof.

29. A method of treating androgen insensitivity syndrome in a subject comprising administering to the subject a therapeutically effective amount of a compound according to claim 1, or its isomer, pharmaceutically acceptable salt, pharmaceutical product, hydrate or any combination thereof.

30. A method of increasing or modulating ovulation in an animal comprising administering to the animal a therapeutically effective amount of a compound according to claim 1, or its isomer, pharmaceutically acceptable salt, pharmaceutical product, hydrate or any combination thereof.

31. A method of treating AR-expressing cancer in a subject comprising administering to the subject a therapeutically effective amount of a compound according to claim 1, or its isomer, pharmaceutically acceptable salt, pharmaceutical product, hydrate or any combination thereof.

32. The method according to claim 31, wherein the cancer is at least one of breast cancer, testicular cancer, cancers associated with partial androgen insensitivity syndromes (PAIS) such as gonadal tumors and seminoma, uterine cancer, ovarian cancer, cancer of the fallopian tubes or peritoneum, salivary gland cancer, bladder cancer, urogenital cancer, brain cancer, skin cancer, lymphoma, mantle cell lymphoma, liver cancer, hepatocellular carcinoma, renal cancer, renal cell carcinoma, osteosarcoma, pancreatic cancer, endometrial cancer, lung cancer, non-small cell lung cancer (NSCLC), gastric cancer, colon cancer, perianal adenoma, or central nervous system cancer.

33. The method according to claim 32, wherein the breast cancer is triple negative breast cancer.

34. A method of reducing the levels of polyglutamine (polyQ) AR polymorphs in a subject comprising administering to the subject a therapeutically effective amount of a compound according to claim 1, or its isomer, pharmaceutically acceptable salt, pharmaceutical product, hydrate or any combination thereof.

35. The method according to claim 34, wherein the polyQ-AR is a short polyQ polymorph or a long polyQ polymorph.

36. The method according to claim 35, wherein the polyQ-AR is a short polyQ polymorph and the method further treats dermal disease.

37. The method according to claim 36, wherein the dermal disease is at least one of alopecia, seborrhea, seborrheic dermatitis, or acne.

38. The method according to claim 34, wherein the polyQ-AR is a long polyQ polymorph and the method further treats Kennedy's disease.

39. A method of treating amyotrophic lateral sclerosis (ALS) in a subject comprising administering to the subject a therapeutically effective amount of a compound according to claim 1, or its isomer, pharmaceutically acceptable salt, pharmaceutical product, hydrate or any combination thereof.

40. A method of treating uterine fibroids in a subject comprising administering to the subject a therapeutically effective amount of a compound according to claim 1, or its isomer, pharmaceutically acceptable salt, pharmaceutical product, hydrate or any combination thereof.

41. A method of treating abdominal aortic aneurysm (AAA) in a subject comprising administering a therapeutically effective amount of a compound according to claim 1, or its isomer, pharmaceutically acceptable salt, pharmaceutical product, hydrate or any combination thereof.

42. A method of treating, suppressing, reducing the incidence, reducing the severity, or inhibiting the progression of a hormonal condition in a male in need thereof, comprising administering to the subject a therapeutically effective amount of a compound according to claim 1, or its isomer, pharmaceutically acceptable salt, pharmaceutical product, hydrate or any combination thereof.

43. The method of claim 42, wherein said condition is hypergonadism, hypersexuality, sexual dysfunction, gynecomastia, precocious puberty in a male, alterations in cognition and mood, depression, hair loss, hyperandrogenic dermatological disorders, precancerous lesions of the prostate, benign prostate hyperplasia, prostate cancer and/or other androgen-dependent cancers.

44. A method of treating or inhibiting the progression of refractory prostate cancer (PCa) or increasing the survival of a male subject suffering from refractory prostate cancer comprising administering to the subject a therapeutically effective amount of a compound according to claim 1, or its isomer, pharmaceutically acceptable salt, pharmaceutical product, hydrate or any combination thereof.

45. A method of treating or increasing the survival of a male subject suffering from castration resistant prostate cancer (CRPC) comprising administering to the subject a therapeutically effective amount of a compound according to claim 1, or its isomer, pharmaceutically acceptable salt, pharmaceutical product, hydrate or any combination thereof.
